(19) United States
(12) Patent Application Publication
(10) Pub. No.: US 2026/0297094 A1
(43) Pub. Date: Oct. 1, 2026

(54) Tetracyclic Compounds as SMARCA2 and/or SMARCA4 Degraders

(71) Applicant: Aurigene Oncology Limited, Bangalore (IN)

(72) Inventors: Chandrasekhar Abbineni, Hyderabad (IN); Susanta Samajdar, Bangalore (IN); Bilash Kuila, Howrah (IN)

(73) Assignee: Aurigene Oncology Limited, Bangalore (IN)

(21) Appl. No.: 19/473,916

(22) PCT Filed: Apr. 10, 2024
(86) PCT No.: PCT/IB2024/053500

§ 371 Date: Oct. 9, 2025

(30) Foreign Application Priority Data
Apr. 12, 2023 (IN) 202341027216

(51) Int. Cl. C07D 487/04; A61K 31/519; A61P 35/00; C07D 471/14

(52) U.S. Cl. CPC C07D 487/04; A61K 31/519; A61P 35/00; C07D 471/14

(57) ABSTRACT

The present invention provides tetracyclic compound of formula (I)

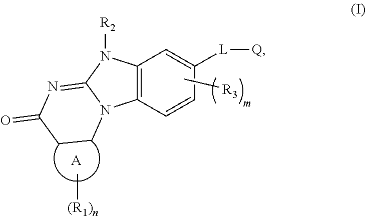

which are therapeutically useful as SMARCA2 and/or SMARCA4 degraders. These compounds are useful in the treatment and/or prevention of diseases or disorders dependent upon SMARCA2 and/or SMARCA4 in a subject. The present invention also provides preparation of the compounds and pharmaceutical compositions of formula (I) or a pharmaceutically acceptable salt or a stereoisomer or a tautomer thereof.

This application claims a benefit of Indian provisional application No. 202341027216, filed on Apr. 12, 2023, the specifications of which are hereby incorporated by reference in their entirety.

### FIELD OF THE INVENTION

The present application relates to tetracyclic compounds and a pharmaceutically acceptable salt or a stereoisomer or a tautomer thereof which are useful as SMARCA2 and/or SMARCA4 degraders and, for the treatment of diseases or disorders dependent on SMARCA2 and/or SMARCA4. The present application also relates to a method of preparation of the said tetracyclic compounds and pharmaceutical compositions comprising the said compounds.

### BACKGROUND OF THE INVENTION

One of the most significant findings from the cancer genome profiling is the discovery of frequent mutations in various subunits of the mammalian SWI/SNF (SWItch/Sucrose Non-Fermentable) chromatin remodelling complex. Approximately 20% of human cancers are associated with somatic mutations in subunits of the SWI/SNF complex, a chromatin remodelling complex that influences gene regulation by disrupting histone-DNA contacts (PNAS Feb. 25, 2014. 111 (8) 3128-3133).

SWI/SNF complexes contain either of two closely related and evolutionarily conserved catalytic ATPase subunits: Brahma (BRM/SMARCA2) or Brahma-related gene 1 (BRG1/SMARCA4). They share approximately 75% identity at the protein level. Although BRG1- and BRM-containing complexes show some redundancy, they may function distinctively. In human cancer, BRG1 seems to be one of the most frequently mutated subunit genes, whereas the BRM gene is rarely mutated. BRG1/SMARCA4 mutations occurring in ~10-15% of lung adenocarcinomas. BRM/SMARCA2 is essential for the growth of tumour cells that harbor loss of function mutations in BRG1/SMARCA4. Depletion of BRM in BRG1-deficient cancer cells leads to a cell cycle arrest, induction of senescence and increased levels of global H3K9me31 (PNAS Feb. 25, 2014. 111 (8) 3128-3133).

In some tumour types, mutations within the SWI/SNF complex lead to context specific vulnerabilities such as the requirement of SMARCA2 for survival of tumour cells lacking SMARCA4. This finding of SMARCA2/4 synthetic lethal relationship translates in vivo which emphasizes SMARCA2 as a promising therapeutic target for the treatment SMARCA4-deficient cancers. Moreover, the SMARCA4-deficient patient population generally lacks targetable oncogenes (such as mutant EGFR or ALK translocations), which further emphasizes the potential of developing SMARCA2 inhibitors. Characterization of SMARCA4 function in tumours with high SMARCA4 levels, shows effects on signalling pathways that result in increased proliferation and survival. SMARCA4 knockdown in tumours that show elevated levels known to inhibit proliferation and other cancer cell properties. Studies have also shown that SMARCA4 knock down/modulation increases sensitivity to known chemotherapeutic agents, thereby indicating that SMARCA4 targeting could also be an adjuvant therapy to existing chemotherapeutic approaches (PNAS Feb. 25, 2014. 111 (8) 3128-3133; J Pathol. 2016 February; 238 (3): 389-400).

Contrary to the point that genetic silencing of SMARCA2 leading to potent anti-proliferative activity in SMARCA4-deficient cancer cell lines, pharmacological studies of PFI-3, a selective cell permeable SMARCA2/4 bromodomain inhibitor which is capable of binding to SMARCA2 and SMARCA4 bromodomain, fail to display an antiproliferative phenotype. This indicates that bromodomain function of SMARCA2/4 is dispensable for tumor cell proliferation, while the catalytic ATPase activity is essential (Cancer Res. 2015 Sep. 15; 75 (18): 3865-3878). In order to mimic the phenotype achieved by genetic silencing, approaches that lead to reduction or complete elimination of SMARCA2/4 may be needed.

The ubiquitin-proteasome system (UPS) is a major pathway that regulates the levels of intracellular proteins and provides a fine balance between protein synthesis and degradation required for normal maintenance of cellular functions, such as proliferation, differentiation and cell death. Ubiquitination is a post-translational modification, where a small protein, ubiquitin, is covalently attached to lysine residues on a substrate protein which is carried out sequentially by a cascade of enzymatic reactions involving an intimate collaboration between E1 activating, E2 conjugating and E3 ligating enzymes and subsequent degradation of the tagged proteins (J. Biosci. 31 (1), March 2006, 137-155; Expert Opin. Ther. Targets. 2013 September; 17 (9): 1091-1108 and Cell Research (2016) 26:484-498).

Proteolysis targeting chimeras (PROTACs) are the heterobifunctional molecules that contain a ligand for a target protein of interest connected via a linker to a ligand for an E3 ubiquitin ligase. Upon such bi-functional molecule-mediated heterodimerization of the two bound proteins, the target protein is ubiquitinated and degraded by the proteasome in cells. Many such bi-functional molecules have been developed to recruit E3 ubiquitin ligases to a variety of substrates using high-affinity ligands for the protein of interest. Proteins effectively degraded using these approaches include RIPK2 and ERRα, BRD4, BRD9, BCR/Abl and Abl and Era. (Cell Chemical Biology 25, 1-10, Jan. 18, 2018). E3 ubiquitin ligases (of which over 600 are known in humans) confer substrate specificity for ubiquitination and are more attractive therapeutic targets than general proteasome inhibitors due to their specificity for certain protein substrates (Cancer Res. 2017 May 1; 77 (9): 2476-2487).

Small molecule ligands targeting the bromodomains of SMARCA2 and SMARCA4 have been reported in the literature (Journal of Medicinal Chemistry 2016, 59, 4800-4811; Hoffman et al., PNAS, 2014b, 777, 3128-3133; Sutherell et al., 2016, Journal of Medicinal Chemistry 59, 5095-5101 and WO2016138114). Although cells lacking SMARCA4 activity are vulnerable to the loss of SMARCA2 (2014a, PNAS 777, 3128-3133). SMARCA2/4 inhibitors have failed to phenocopy these anti-proliferative effects. In agreement with this, re-expression of SMARCA2 variants in cells, where the endogenous protein had been suppressed, showed that an intact bromodomain is not required to maintain proliferation (Cancer Res. 2015 September 15; 75 (18): pp. 3865-3878). SMARCA2/4 Bromo domain inhibitors are thus precluded from use for the treatment of SMARCA4 mutant cancers but could provide attractive ligands for PROTAC conjugation.

It is therefore reasoned that a PROTAC targeting the non-functional bromodomain of SMARCA2 and/or SMARCA4 should offer an opportunity to exploit the vulnerability of SMARCA2 in SMARCA4 mutated cancer cells for therapeutic purposes. The principle of conjugation of a suitable SMARCA ligand with an E3 ligase binder has been described in WO2016/105518, WO2017007612, WO2017011371, WO2020251974, WO2021133920, WO2021133917, WO2022240825, WO2021252666, WO2022099117, WO2023018648, WO2023278402 and WO2023052363. However, there remains a need to find compounds that are SMARCA degraders useful as therapeutic agents.

### SUMMARY OF THE INVENTION

Provided herein tetracyclic compounds and pharmaceutical compositions thereof that are useful as SMARCA2 and/or SMARCA4 degraders and for treating diseases or conditions or disorders that are dependent upon or mediated by SMARCA2 and/or SMARCA4.

In one aspect, the present application provides compound of formula (I):

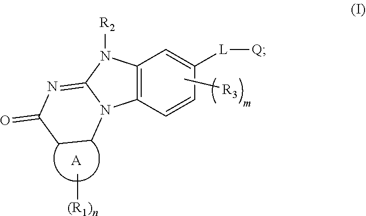

or a pharmaceutically acceptable salt or a stereoisomer or a tautomer thereof;
wherein,
   A is phenyl or pyridyl;
   each R₁ and R₃ independently is halogen, (C₁-C₆)alkyl, (C₁-C₆)alkoxy, amino, halo(C₁-C₆)alkyl, hydroxy, 3- to 8-membered cycloalkyl, hydroxy (C₁-C₆)alkyl or cyano;
   R₂ is hydrogen, halogen, hydroxy, (C₁-C₆)alkyl or 3- to 8-membered cycloalkyl; wherein the alkyl and cycloalkyl is substituted with 0, 1, 2 or 3 substituents, independently, selected from hydroxy, halogen and alkoxy;
   L is

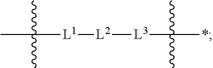

wherein asterisk mark represents the point of attachment with Q;
L¹ and L³ independently is a bond, (C₁-C₆)alkylenyl, 6- to 10-membered arylenyl or 4-6-membered heterocycloalkylenyl; wherein the alkylenyl, arylenyl and to heterocycloalkylenyl, at each occurrence, independently is substituted with 0, 1, 2, 3 or 4 of R₇;
L² is (C₁-C₆)alkylenyl or 4- to 6-membered heterocycloalkylenyl substituted with 0, 1, 2, 3 or 4 of R₇;
each R₇ is halogen, hydroxy, (C₁-C₆)alkyl, (C₁-C₆)alkoxy or halo(C₁-C₆)alkyl;
Q is represented by formula Q1, Q2 or Q3:

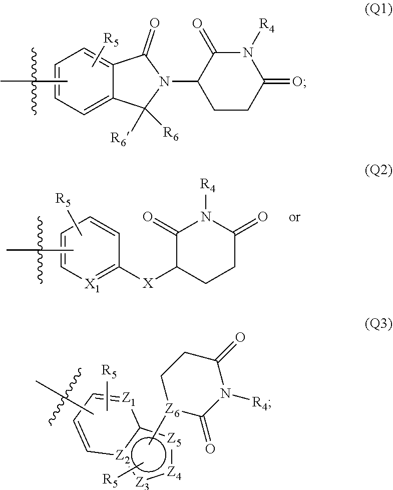

wherein,
   X is a bond, O, —(CO)—NH— or —NH—;
   X₁ is N or CH;
   each Z₁ and Z₆ is independently C, CH or N;
   each Z₂, Z₃ Z₄ and Z₅ is independently C, CH or N;
   each R₄ is hydrogen or (C₁-C₆)alkyl;
   each R₅ is hydrogen, halogen, halo(C₁-C₆)alkyl or (C₁-C₆)alkyl;
   R₆ and R₆′ independently is hydrogen; or R₆ and R₆′ together represent an oxo group; and
   ‘n’ and ‘m’ are an integer, independently selected from 0, 1, 2 and 3.

In yet another aspect, the present application relates to the preparation of compound of formula (I).

In another aspect, the present application provides a pharmaceutical composition comprising a compound of formula (I) or a pharmaceutically acceptable salt or a stereoisomer or a tautomer thereof and a pharmaceutically acceptable carrier or an excipient.

In still another aspect, the present application provides a pharmaceutical composition comprising a compound of formula (I) or a pharmaceutically acceptable salt or a stereoisomer or a tautomer thereof for treating diseases or conditions or disorders that are dependent upon or mediated by SMARCA2 and/or SMARCA4.

In another aspect, the present application provides a pharmaceutical composition comprising a compound of formula (I) or a pharmaceutically acceptable salt or a stereoisomer or a tautomer thereof for treating diseases or conditions or disorders that have altered SMARCA2 and/or SMARCA4 including mutations and overexpression.

In another aspect, the present application provides a pharmaceutical composition comprising a compound of formula (I) or a pharmaceutically acceptable salt or a stereoisomer or a tautomer thereof for treating diseases or conditions or disorders wherein degradation of SMARCA2 and/or SMARCA4 proteins provide a benefit, e.g., cancer.

In another aspect, the present application provides a composition of a compound of formula (I) or a pharmaceutically acceptable salt or a stereoisomer or a tautomer thereof for treating diseases or conditions or disorders that are dependent upon altered activity of SWI/SNF complex with or without chromatic remodeling activities.

In another aspect, the present application provides methods of treating a condition or a disease or a disorder by administering a therapeutically effective amount of a compound of formula (I) or a pharmaceutically acceptable salt or a stereoisomer or a tautomer thereof to an individual, e.g., a human, in need thereof. The conditions or diseases or disorders of interest is treatable by degradation of SMARCA2 and/or SMARCA4, for example, a cancer, a chronic autoimmune disorder, an inflammatory condition, a proliferative disorder, sepsis or a viral infection.

In another aspect, the present application provides a use of a compound of formula (I) or a pharmaceutically acceptable salt or a stereoisomer or a tautomer thereof for the manufacture of a medicament for treating a disease or a condition or a disorder of interest, e.g., cancer.

### DETAILED DESCRIPTION OF THE INVENTION

The present invention provides tetracyclic compounds, referred as a compound of formula (I), which are useful as SMARCA2 and/or SMARCA4 degraders and for the treatment of conditions dependent on or mediated by SMARCA2 and/or SMARCA4. The present invention further provides pharmaceutical compositions comprising the said compound or a stereoisomer or a tautomer thereof as therapeutic agents.

Each embodiment is provided by way of explanation of the invention and not by way of limitation of the invention. In fact, it will be apparent to those skilled in the art that various modifications and variations can be made to the compounds, compositions and methods described herein without departing from the scope or spirit of the invention. For instance, features illustrated or described as part of one embodiment can be applied to another embodiment to yield a still further embodiment. Thus, it is intended that the present invention includes such modifications and variations and their equivalents. Other objects, features and aspects of the present invention are disclosed in or are obvious from, the following detailed description. It is to be understood by one of ordinary skill in the art that the present discussion is a description of exemplary embodiments only and is not to be construed as limiting the broader aspects of the present invention.

In one embodiment, the present invention provides compound of formula (I),

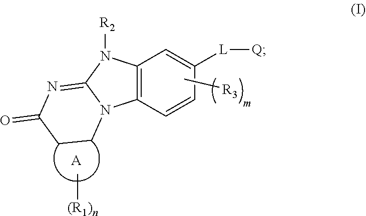

or a pharmaceutically acceptable salt or a stereoisomer or a tautomer thereof;
wherein,
   A is phenyl or pyridyl;
   each R₁ and R₃ independently is halogen, (C₁-C₆)alkyl, (C₁-C₆)alkoxy, amino, halo(C₁-C₆)alkyl, hydroxy, 3- to 8-membered cycloalkyl, hydroxy (C₁-C₆)alkyl or cyano;
   R₂ is hydrogen, halogen, hydroxy, (C₁-C₆)alkyl or 3- to 8-membered cycloalkyl; wherein the alkyl and cycloalkyl is substituted with 0, 1, 2 or 3 substituents, independently, selected from hydroxy, halogen and alkoxy;
   L is

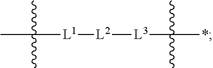

wherein asterisk mark represents the point of attachment with Q;
L¹ and L³ independently is a bond, (C₁-C₆)alkylenyl, 6- to 10-membered arylenyl or 4- to 6-membered heterocycloalkylenyl; wherein the alkylenyl, arylenyl and heterocycloalkylenyl, at each occurrence, independently is substituted with 0, 1, 2, 3 or 4 of R₇;
L² is (C₁-C₆)alkylenyl or 4- to 6-membered heterocycloalkylenyl substituted with 0, 1, 2, 3 or 4 R₇;
each R₇ is halogen, hydroxy, (C₁-C₆)alkyl, (C₁-C₆)alkoxy or halo(C₁-C₆)alkyl; Q is represented by formula Q1, Q2 or Q3:

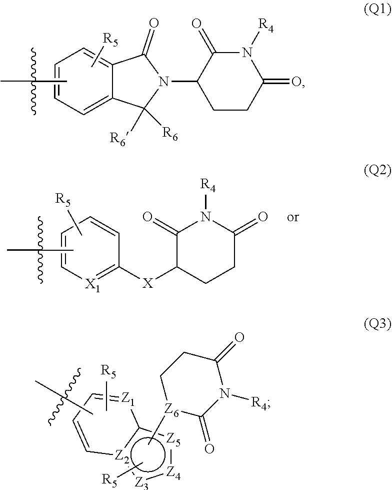

wherein,
   X is a bond, O, —(CO)—NH— or —NH—;
   X₁ is N or CH;
   each Z₁ and Z₆ is independently C, CH or N;
   each Z₂, Z₃ Z₄ and Z₅ is independently C, CH or N;
   each R₄ is hydrogen or (C₁-C₆)alkyl;
   each R₅ is hydrogen, halogen, halo(C₁-C₆)alkyl or (C₁-C₆)alkyl;
   R₆ and R₆′ independently is hydrogen; or R₆ and R₆′ together represent an oxo group; and
   ‘n’ and ‘m’ are an integer, independently selected from 0, 1, 2 and 3.

In one embodiment, A is phenyl.

In one embodiment, A is pyridyl.

In one embodiment, R₁ and R₃ independently is halogen, (C₁-C₆)alkyl, (C₁-C₅)alkoxy, amino, halo(C₁-C₆)alkyl, 3- to 8-membered cycloalkyl, or hydroxy.

In one embodiment, R₁ and R₃ independently is halogen, (C₁-C₆)alkyl, (C₁-C₆)alkoxy, halo(C₁-C₆)alkyl, amino or hydroxy.

In one embodiment, R₁ and R₃ independently is halogen, amino or hydroxy.

In one embodiment, R₁ and R₃ independently is (C₁-C₆)alkyl, (C₁-C₆)alkoxy or halo(C₁-C₆)alkyl.

In one embodiment, R₁ and R₃ independently is selected from —Cl, —Br, —CH₃, —CH₂—CH₃, —CH(CH₃)—CH₃, —CF₂, —CF₃, —O—CH₂—CH₃, —O—CH(CH₃)—CH₃ and —NH₂.

In one embodiment, R₁ and R₃ independently is selected from cyclopropyl, cyclobutyl and cyclopentyl.

In one embodiment, R₁ is —Br, —Cl, —OH or —NH₂.

In one embodiment, R₃ is —Br, —Cl, —OH or —NH₂.

In one embodiment, L is

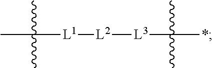

wherein asterisk mark represents the point of attachment with Q.

In one embodiment, L¹ is a bond, 6- to 10-membered arylenyl or 4- to 6-membered heterocycloalkylenyl; wherein arylenyl and heterocycloalkylenyl, at each occurrence, independently is substituted with 0, 1, 2, 3 or 4 of R⁷.

In one embodiment, L¹ is a bond.

In one embodiment, L¹ is a bond, phenylenyl or 4- to 6-membered heterocycloalkylenyl; wherein phenylenyl and heterocycloalkylenyl, at each occurrence, independently is substituted with 0, 1, 2, 3 or 4 of R₇;

L² is —CH₂— or 4- to 6-membered heterocycloalkylenyl substituted with 0, 1, 2, 3 or 4 of R₇; and

L³ is a bond, (C₁-C₆)alkylenyl or 4- to 6-membered heterocycloalkylenyl; wherein the alkylenyl, and the heterocycloalkylenyl, at each occurrence, is substituted with 0, 1, 2, 3 or 4 R₇.

In one embodiment, L³ is a bond or 4- to 6-membered heterocycloalkylenyl.

In one embodiment, L³ is a bond.

In one embodiment, L³ is a bond or (C₁-C₆)alkylenyl; wherein the alkylenyl, at each occurrence, is substituted with 0, 1, 2, 3 or 4 R₇.

In one embodiment, L³ is 4- to 6-membered heterocycloalkylenyl.

In one embodiment, L² is (C₁-C₆)alkylenyl or 4- to 6-membered heterocycloalkylenyl, which is substituted with 0, 1, 2, 3 or 4 of R₇.

In one embodiment, L² is —CH₂— or 4- to 6-membered heterocycloalkylenyl, which is substituted with 0, 1, 2, 3 or 4 of R₇.

In one embodiment, L² is 4- to 6-membered heterocycloalkylenyl, which is substituted with 0, 1, 2, 3 or 4 of R₇.

In one embodiment, L² is (C₁-C₆)alkylenyl.

In one embodiment, L² is —CH₂—.

In one embodiment, L² is 6-membered heterocycloalkylenyl, which is substituted with 0, 1 or 2 of R₇.

In one embodiment, L² is attached to Q, if L³ is bond.

In any one of the preceding embodiment, 4- to 6-membered heterocycloalkylenyl is selected from azetidinylenyl, pyrrolidinylenyl, piperidinylenyl, piperazinylenyl, tetrahydropyranylenyl, morpholinylenyl, thiomorpholinylenyl, 1,4-dioxanylenyl and tetrahydropyranylenyl.

In one embodiment, L¹, L² and L³ are independently 4- to 6-membered heterocycloalkylenyl, wherein heterocycloalkylenyl is selected from piperidinylenyl and piperazinylenyl.

In one embodiment, L¹ is a bond or a group selected from

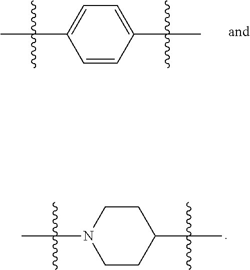 [SMILES: *c1ccc(*)cc1 ; *C1CCN(*)CC1]

In one embodiment, L² is selected from

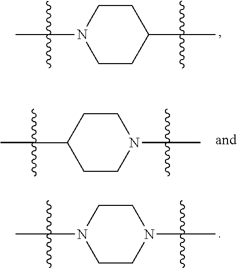

In one embodiment, L³ is a bond or a group selected from —CH₂—, —CH₂—CH₂—, —CH(CH₃)—CH₂—, —CH₂—CH(CH₃)—CH₂—,

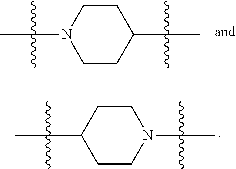

In one embodiment, L¹ and L³ independently is a bond.

In one embodiment, L is selected from the group

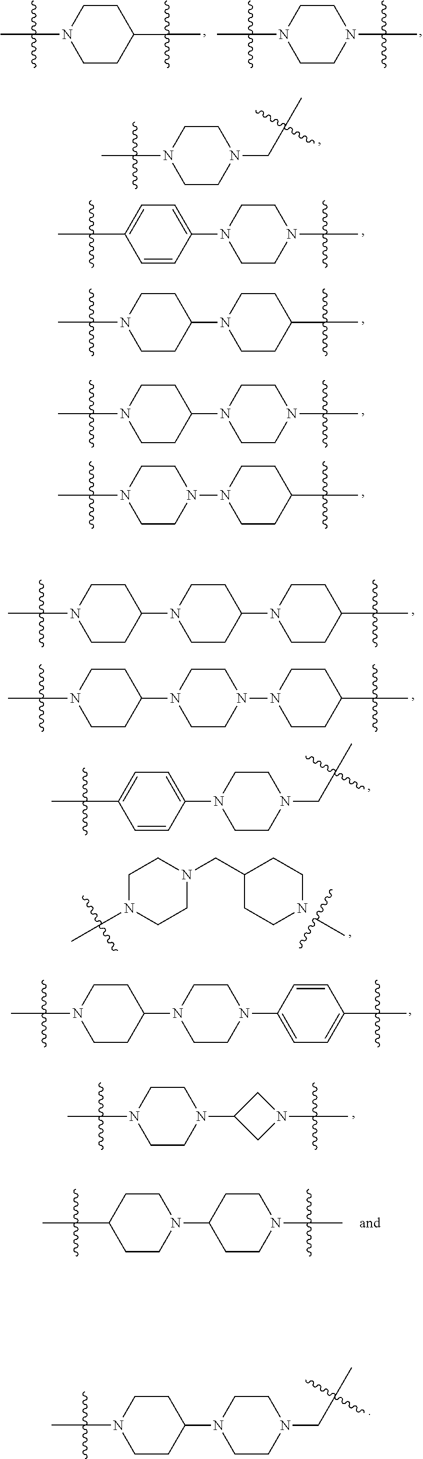

In one embodiment, R₂ is hydrogen, (C₁-C₆)alkyl or 3- to 8-membered cycloalkyl; wherein the alkyl and cycloalkyl independently is substituted with 0, 1, 2 or 3 substituents selected from hydroxy, halogen or (C₁-C₆)alkoxy;

In one embodiment, R₂ is hydrogen or halogen.

In one embodiment, R₂ is (C₁-C₆)alkyl.

In one embodiment, R₂ is 3- to 8-membered cycloalkyl, which is selected from cyclopropyl, cyclobutyl, cyclopentyl and cyclohexyl.

In one embodiment, R₂ is hydrogen, —CH₃, —CH(CH₃)₂, cyclopropyl, cyclobutyl or cyclopentyl.

In one embodiment, R₂ is hydrogen, cyclopropyl or cyclopentyl.

In one embodiment, R₂ is hydrogen, —CH₃ or —CH(CH₃)₂.

In one embodiment, R₇ is halogen.

Q is represented by formula Q1, Q2 or Q3:

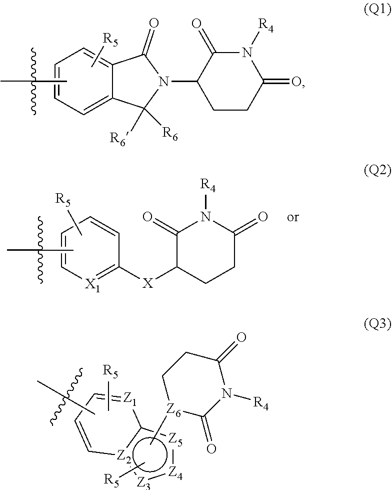

wherein,
   X is a bond, —O—, —(CO)—NH— or —NH—;
   X₁ is N or CH;
   each Z₁ and Z₆ is independently C, CH or N;
   each Z₂, Z₃ Z₄ and Z₅ is independently C, CH or N; wherein at least one of Z₃ and Z₄ is C;
   each R₄ is hydrogen or (C₁-C₆)alkyl,
   each R₅ is hydrogen, halogen, halo(C₁-C₆)alkyl or (C₁-C₆)alkyl; and R₆ and R₆′ independently is hydrogen; or R₆ and R₆′ together represent an oxo group.

In one embodiment, R₅ is hydrogen, halogen or (C₁-C₆)alkyl.

In one embodiment, R₅ is hydrogen.

In one embodiment, R₄ is hydrogen or (C₁-C₆)alkyl.

In one embodiment, Q is represented by formula Q1:

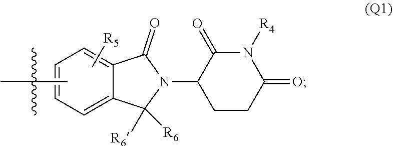

wherein
   R₅ is hydrogen;
   R₆ and R₆′ independently is hydrogen; or R₆ and R₆′ together represent an oxo group; and
   R₄ is hydrogen or (C₁-C₆)alkyl.

In one embodiment, Q is represented by formula Q2:

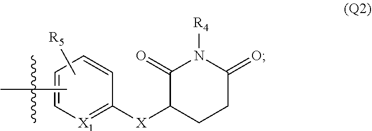

wherein
   X is a bond, —O—, —(CO)—NH— or —NH—;
   X₁ is N or CH;
   R₅ is hydrogen or halogen; and
   R₄ is hydrogen or (C₁-C₆)alkyl.

In one embodiment, Q is represented by formula Q3:

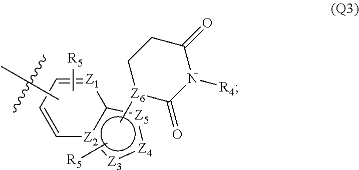

Wherein,
   R₄ is hydrogen or (C₁-C₆)alkyl;
   R₅ is hydrogen or halogen;
   each Z₁ and Z₆ is independently C or N; and
   each Z₂, Z₃ Z₄ and Z₅ is independently C or N; wherein at least one of Z₃ and Z₄ is C.

In one embodiment, R₆ and R₆′ independently is hydrogen.

In one embodiment, R₆ and R₆′ together represent an oxo group.

In one embodiment, R₄ is hydrogen.

In one embodiment, X is a bond.

In one embodiment, X is —O—, —(CO)—NH— or —NH—.

In one embodiment, X₁ is CH.

In one embodiment, ‘n’ is an integer selected from 0, 1, 2 and 3.

In one embodiment, ‘m’ is an integer selected from 0, 1, 2 and 3.

In one embodiment, ‘n’ is an integer selected from 0, 1 and 2.

In one embodiment, ‘m’ is an integer selected from 0, 1 and 2.

In one embodiment, ‘n’ is an integer selected from 0 and 1.

In one embodiment, ‘m’ is an integer selected from 0 and 1.

In one embodiment, Q represents

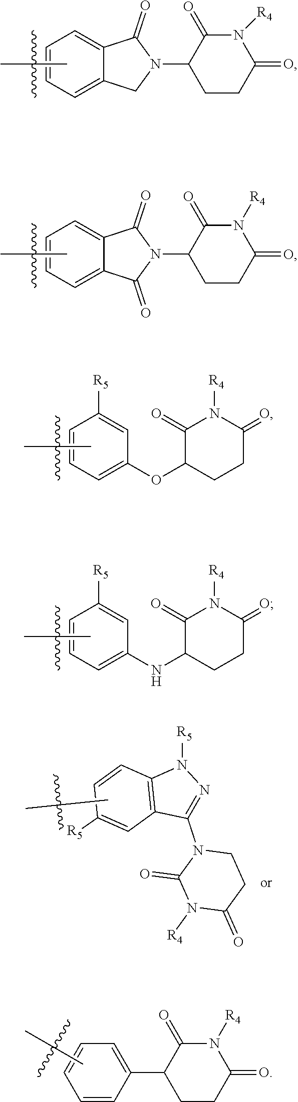

In one embodiment, Q is selected from,

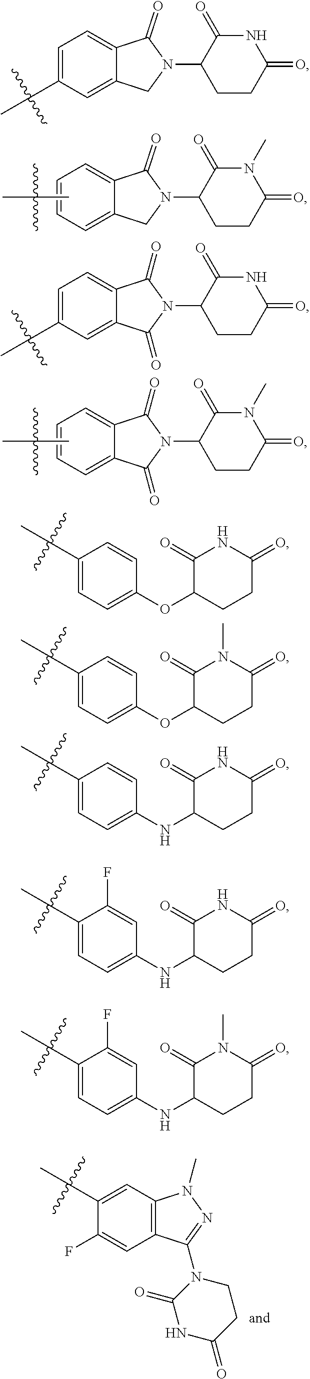

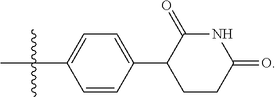 [SMILES: *c1ccc(C2CCC(=O)NC2=O)cc1]

In one embodiment, the group

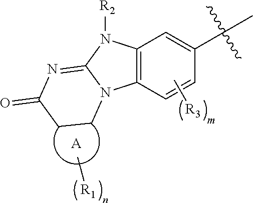

represents

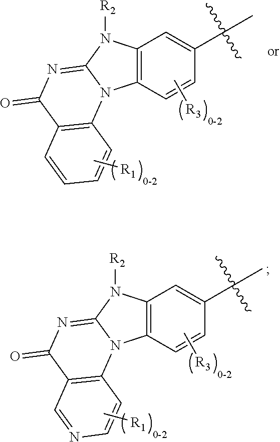

wherein each R₁ and R₃ independently is halogen, (C₁-C₆)alkyl, (C₁-C₆)alkoxy, amino, 3- to 8-membered cycloalkyl, or hydroxy.

In one embodiment of compound of formula (I), wherein
A is phenyl or pyridyl;
each R₁ and R₃ independently is selected from halogen, (C₁-C₆)alkyl, (C₁-C₆)alkoxy, halo(C₁-C₆)alkyl, amino and hydroxy;
R₂ is hydrogen, —CH₃, —CH(CH₃)₂, cyclopropyl, cyclobutyl or cyclopentyl;
Q represents

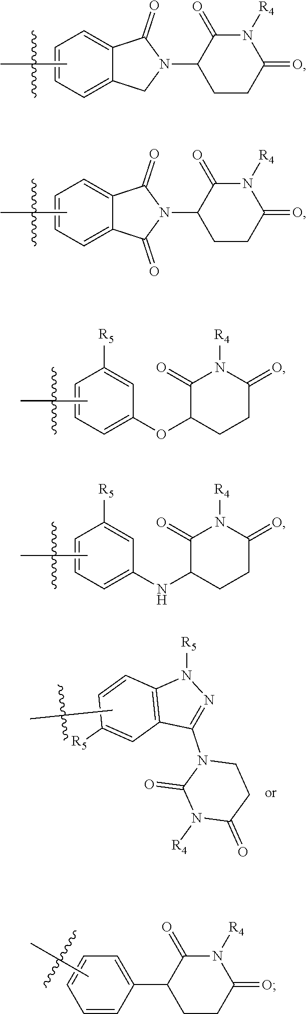

L is

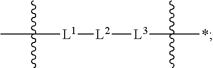

wherein asterisk mark represents the point of attachment with Q; wherein L¹, if present, is attached to fused phenyl ring and L³, if present, is attached to Q;
L¹ is a bond, phenylenyl or 4- to 6-membered heterocycloalkylenyl; wherein phenylenyl and heterocycloalkylenyl, at each occurrence, independently is substituted with 0, 1, 2, 3 or 4 of R₇;
L³ is a bond, alkylenyl or 4- to 6-membered heterocycloalkylenyl; wherein the alkylenyl, and the heterocycloalkylenyl, at each occurrence, is substituted with 0, 1, 2, 3 or 4 of R₇;
each R₅ is hydrogen, halogen, halo(C₁-C₆)alkyl or (C₁-C₆)alkyl; and
each R₄ is hydrogen or (C₁-C₆)alkyl.

In one embodiment of compound of formula (I), wherein
A is phenyl or pyridyl;
R₁ and R₃ independently is selected from —Cl, —Br, —CH₃, —CH₂—CH₃, —CH(CH₃)—CH₃, —CF₂, —CF₃, —O—CH₂—CH₃, —O—CH(CH₃)—CH₃, —OH and —NH₂
L is selected from -piperazinylenyl-, -piperazinylenyl-azitidinylenyl-, -piperidinylenyl-piperidinylenyl-, -piperidinylenyl-piperazinylenyl-, -piperidinylenyl-piperidinylenyl-piperidinylenyl-, -piperazinylenyl-alkylenyl-, -phenylenyl-piperidinylenyl-, -phenylenyl-piperazinylenyl-alkylenyl-, -phenylenyl-piperidinylenyl-alkylenyl- and -piperidinylenyl-piperazinylenyl-alkylenyl-; wherein each group is substituted with 0, 1 or 2 substituents selected from halogen, hydroxy, (C₁-C₆)alkyl, (C₁-C₆)alkoxy and halo(C₁-C₆)alkyl;
R₂ is hydrogen, —CH₃, —CH(CH₃)₂, cyclopropyl, cyclobutyl or cyclopentyl;
Q represents

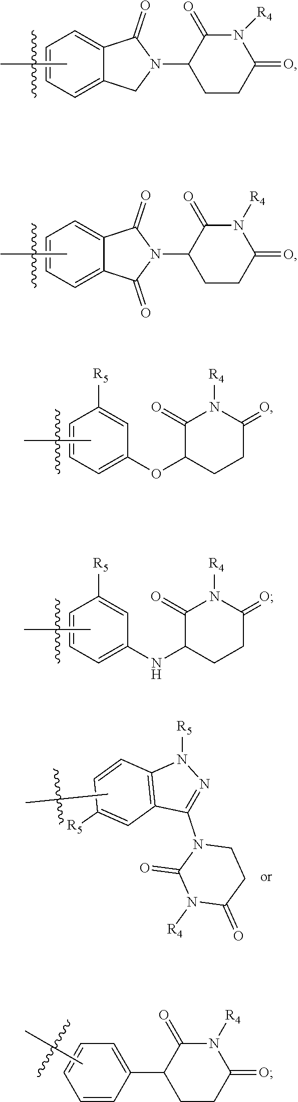

each R₅ is hydrogen, halogen, halo(C₁-C₆)alkyl or (C₁-C₆)alkyl; and
each R₄ is hydrogen or (C₁-C₆)alkyl.

In one embodiment of compound of formula (I), wherein
A is phenyl or pyridyl; wherein each ring is substituted with 0, 1 or 2 R₁ group(s);
R₁ and R₃ independently is selected from —Cl, —Br, —CH₃, —CH₂—CH₃, —CH(CH₃)—CH₃, —CF₂, —CF₃, —O—CH₂—CH₃, —O—CH(CH₃)—CH₃, —OH and —NH₂;
R₂ is hydrogen, —CH₃, —CH(CH₃)₂, cyclopropyl, cyclobutyl or cyclopentyl;
L is selected from the group

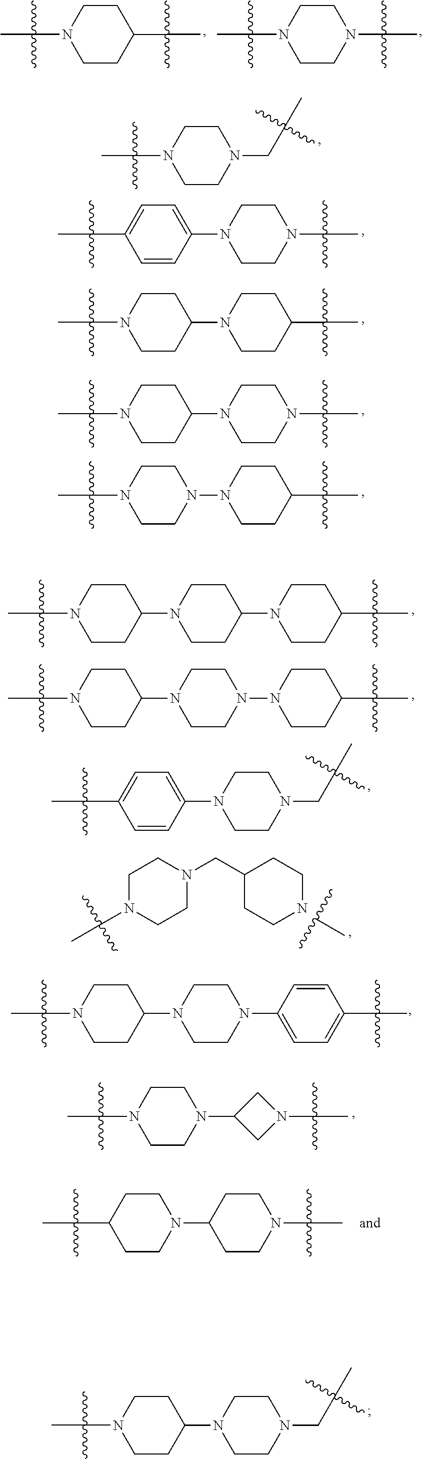

and
Q is selected from

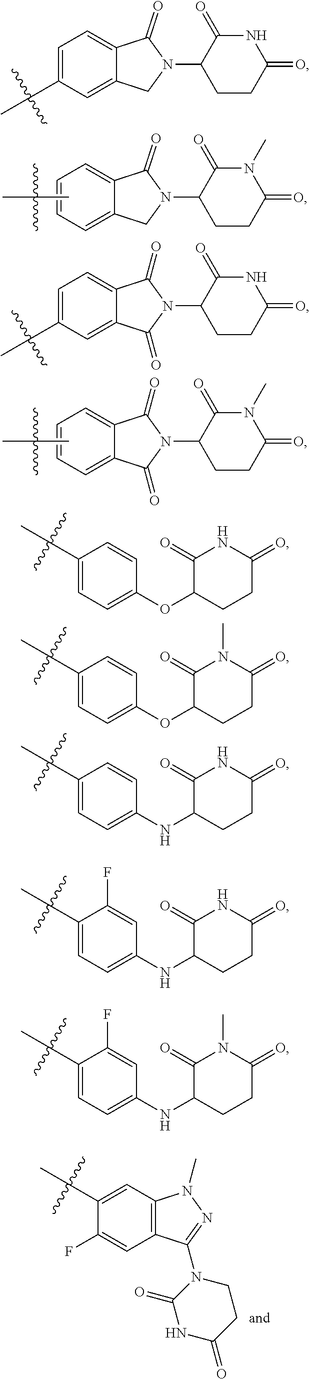

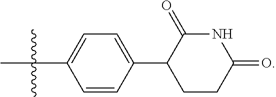 [SMILES: *c1ccc(C2CCC(=O)NC2=O)cc1]

In one embodiment, the present invention provides compound of formula (IA) or a pharmaceutically acceptable salt or a stereoisomer or a tautomer thereof:

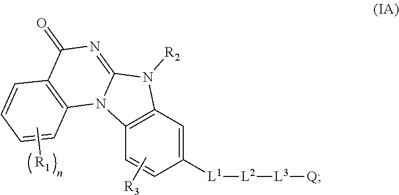

wherein R₁, R₂, R₃, L¹, L², L³, Q and n are as defined in compound of formula (I).

In one embodiment, the compound of formula (IA), wherein R₁ and R₃ independently is halogen, (C₁-C₆)alkyl, (C₁-C₆)alkoxy, halo(C₁-C₆)alkyl, amino, 3- to 8-membered cycloalkyl, or hydroxy.

In one embodiment, the compound of formula (IA), wherein R₁ and R₃ independently is (C₁-C₆)alkyl, (C₁-C₆)alkoxy or halo(C₁-C₆)alkyl.

In one embodiment, the compound of formula (IA), wherein R₁ and R₃ independently is selected from —CH₃, —CH₂—CH₃, —CH(CH₃)—CH₃, —CF₂, —CF₃, —O—CH₂—CH₃, —O—CH(CH₃)—CH₃, —OH and —NH₂.

In one embodiment, the of compound of formula (IA), wherein R₁ and R₃ independently is selected from —Cl, —Br, —OH and —NH₂.

In one embodiment, the compound of formula (IA), wherein R₁ and R₃ independently is selected from —CH₃, —CH₂—CH₃, —CF₂ and —CF₃.

In one embodiment of compound of formula (IA),
L¹ is a bond, phenylenyl or 4- to 6-membered heterocycloalkylenyl;
L² is —CH₂— or 4- to 6-membered heterocycloalkylenyl; and
L³ is a bond, (C₁-C₆)alkylenyl or 4- to 6-membered heterocycloalkylenyl.

In one embodiment of compound of formula (IA), L¹ is a bond.

In one embodiment of compound of formula (IA), L¹ is a bond or a group selected from

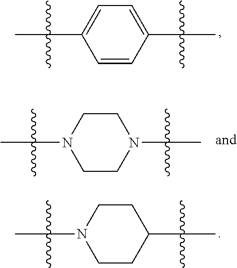

In one embodiment of compound of formula (IA), L¹ is a bond or a group selected from

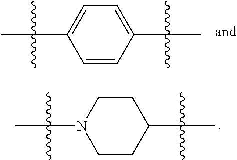

In one embodiment of compound of formula (IA), L² is selected from

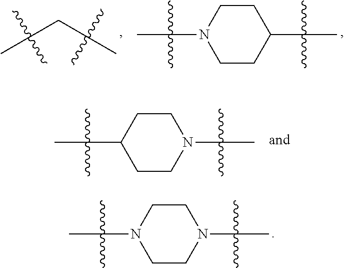

In one embodiment of compound of formula (IA), L³ is selected from —CH₂—, —CH₂—CH₂—, —CH(CH₃)—CH₂—, —CH₂—CH(CH₃)—CH₂—,

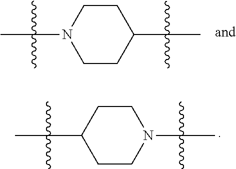

In one embodiment of compound of formula (IA), L³ is selected from —CH₂—, —CH₂—CH₂— and —CH(CH₃)—CH₂—.

In one embodiment, L² is selected from

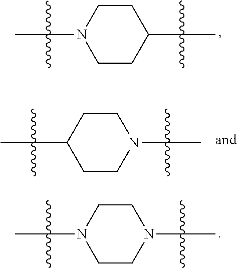

In one embodiment of compound of formula (IA), wherein the group

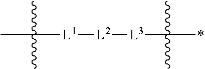

represents

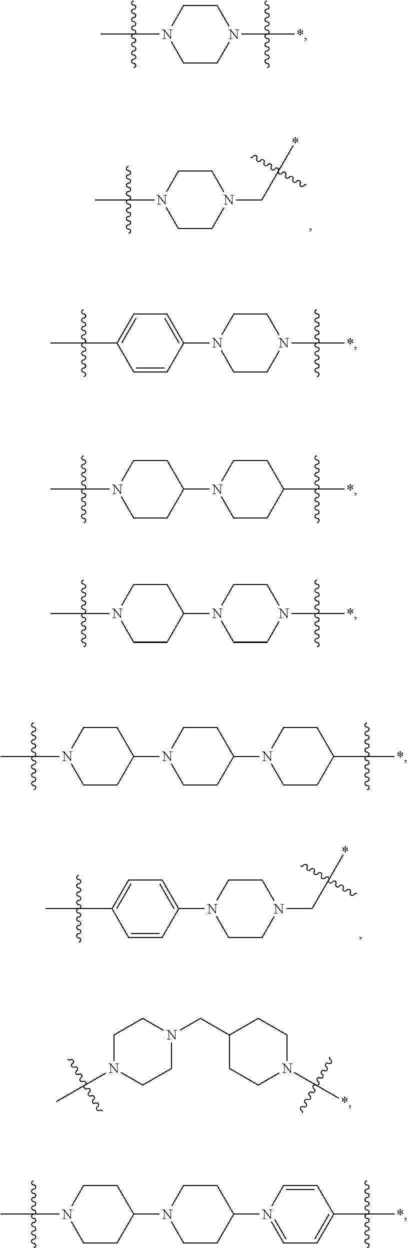

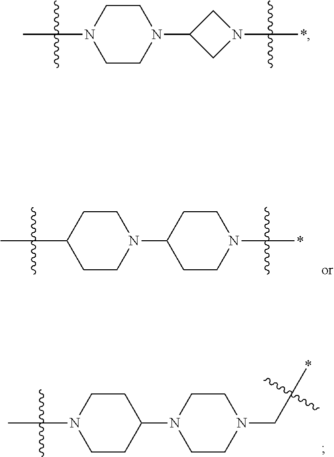

wherein asterisk mark represents the point of attachment with Q.

In one embodiment of compound of formula (IA), wherein the group

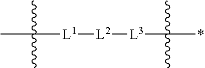

represents

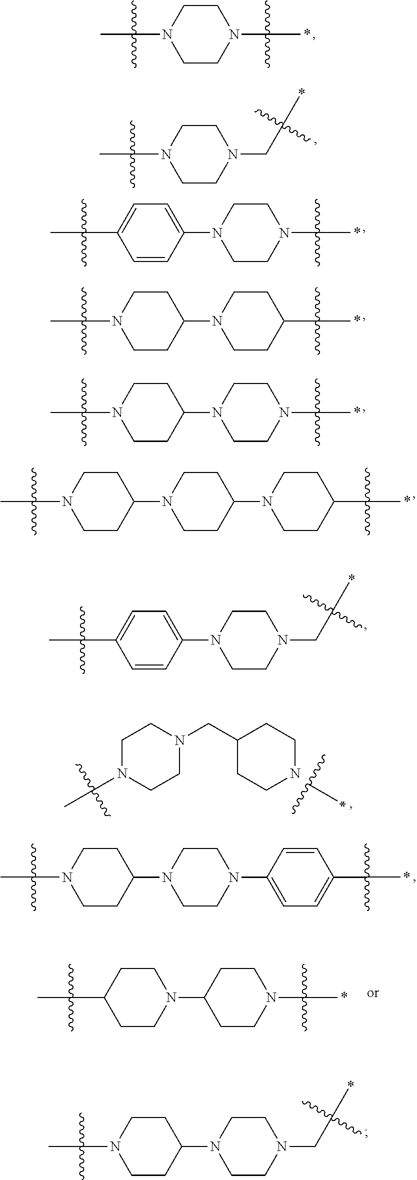

wherein asterisk mark represents the point of attachment with Q.

In one embodiment of compound of formula (IA), Q represents

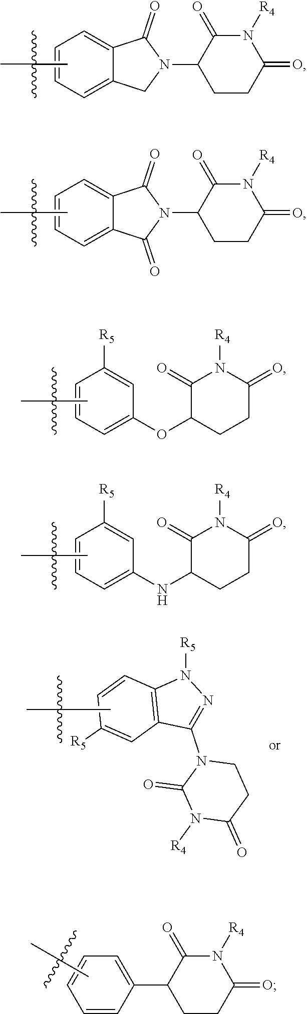

wherein each R₄ is (C₁-C₆)alkyl or hydrogen; and each R₅ is hydrogen or halogen.

In one embodiment of compound of formula (IA), Q is selected from

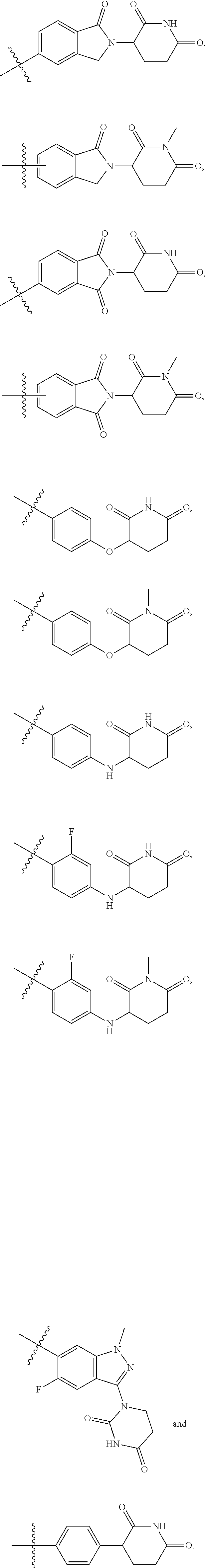

In one embodiment, the present invention provides the compound of formula (IA) or a pharmaceutically acceptable salt or a stereoisomer or a tautomer thereof, wherein
R₁ and R₃ independently is halogen, (C₁-C₆)alkyl, (C₁-C₆)alkoxy, halo(C₁-C₆)alkyl, amino, cyclopropyl, cyclobutyl, cyclopentyl or hydroxy;
R₂ is hydrogen, halogen, hydroxy, (C₁-C₆)alkyl or 3- to 8-membered cycloalkyl;
L¹ is a bond, phenylenyl or 4- to 6-membered heterocycloalkylenyl;
L² is —CH₂— or 4- to 6-membered heterocycloalkylenyl;
L³ is a bond, (C₁-C₆)alkylenyl or 4- to 6-membered heterocycloalkylenyl;
Q is selected from

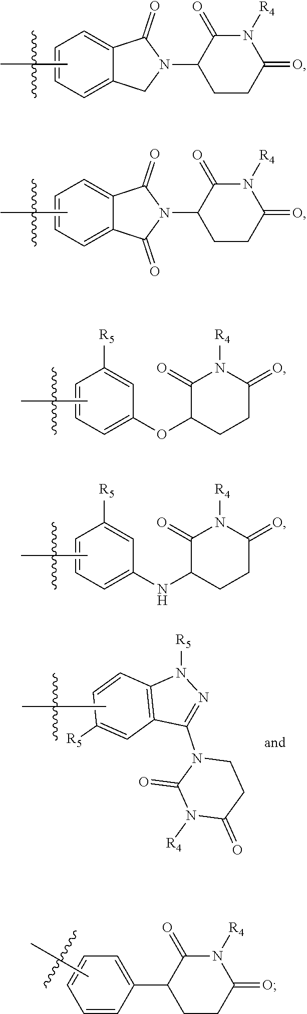

wherein each R₄ is (C₁-C₆)alkyl or hydrogen; and each R₅ is (C₁-C₆)alkyl or hydrogen; and
‘n’ is an integer selected from 0, 1, 2 and 3.

In one embodiment of compound of formula (IA), R₁ and R₃ independently is selected from —Br, —Cl, —CH₃, —CH₂—CH₃, —CH(CH₃)—CH₃, —O—CH₃, —O—CH₂—CH₃, —O—CH(CH₃)—CH₃, —OH, —CF₃, —CF₂ and —NH₂;
R₂ is hydrogen, —CH₃, —CH(CH₃)₂, cyclopropyl, cyclobutyl or cyclopentyl;
L is

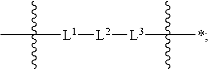

is selected from the group

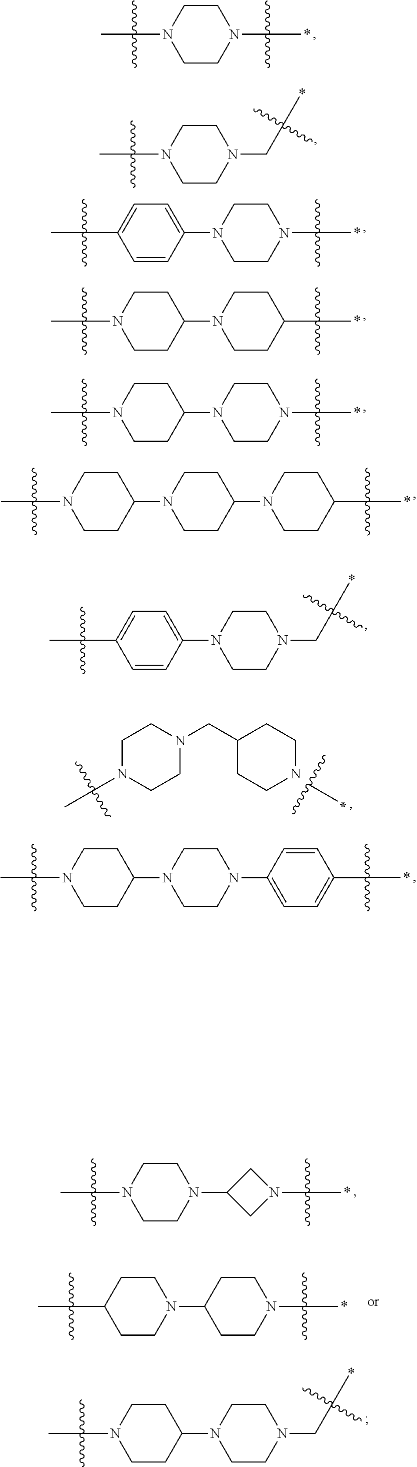

wherein asterisk mark represents the point of attachment with Q.
Q is selected from

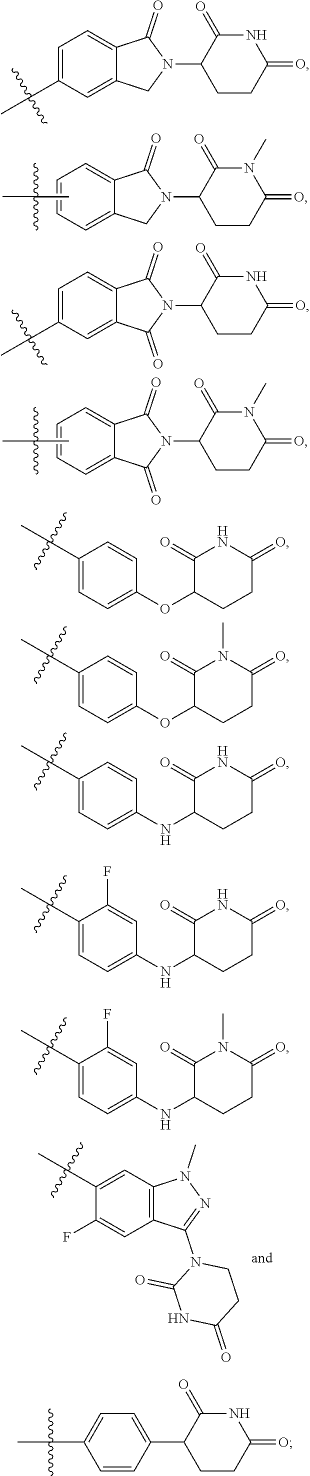

and
‘n’ is an integer selected from 0, 1, 2 and 3.

In one embodiment, the present invention provides compound of formula (IB) or a pharmaceutically acceptable salt or a stereoisomer or a tautomer thereof:

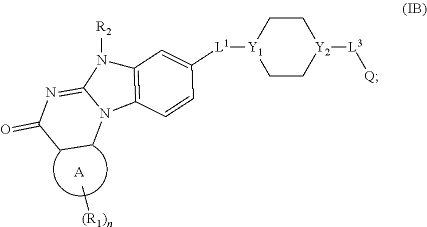

wherein Y₁ and Y₂ independently is CH or N;
L¹ is a bond, phenylenyl or heterocycloalkylenyl;
L³ is a bond, (C₁-C₆)alkylenyl or heterocycloalkylenyl; and
R₁, R₂, Q and n are as defined in compound of formula (I)

In one embodiment of compound of formula (IB), A is phenyl or pyridyl.

In one embodiment of compound of formula (IB), A is phenyl, which is substituted with halogen, (C₁-C₄)alkyl, (C₁-C₄)alkoxy, halo(C₁-C₄)alkyl, amino or hydroxy.

In one embodiment of compound of formula (IB), R₁ is halogen, (C₁-C₆)alkyl, (C₁-C₆)alkoxy, amino, halo(C₁-C₆)alkyl or hydroxy.

In one embodiment of compound of formula (IB), R₁ is (C₁-C₆)alkyl, (C₁-C₆)alkoxy or halo(C₁-C₆)alkyl.

In one embodiment of compound of formula (IB), R₁ is halogen, amino or hydroxy.

In one embodiment of compound of formula (IB), R₁ is selected from —Br, —Cl, —CH₃, —CH₂—CH₃, —CH(CH₃)—CH₃, —CF₂, —CF₃, —O—CH₃, —O—CH₂—CH₃, —O—CH(CH₃)—CH₃, —OH and —NH₂.

In one embodiment of compound of formula (IB), R₂ is hydrogen, halogen, hydroxy, (C₁-C₆)alkyl or 3- to 8-membered cycloalkyl.

In one embodiment of compound of formula (IB), R₂ is (C₁-C₆)alkyl or 3- to 8-membered cycloalkyl.

In one embodiment of compound of formula (IB), R₂ is hydrogen, —CH₃, —CH(CH₃)₂, cyclopropyl, cyclobutyl or cyclopentyl.

In one embodiment of compound of formula (IB), L¹ is a bond or a group selected from

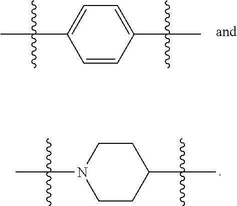 [SMILES: *c1ccc(*)cc1 ; *C1CCN(*)CC1]

In one embodiment of compound of formula (IB), L¹ is a bond.

In one embodiment of compound of formula (IB), L¹ is

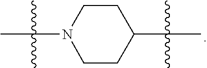 [SMILES: *C1CCN(*)CC1]

In one embodiment of compound of formula (IB), L³ is a bond or a group selected from —CH₂—, —CH₂—CH₂—, —CH(CH₃)—CH₂—,

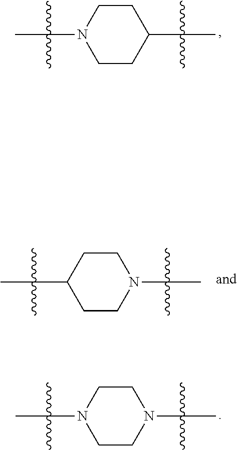 [SMILES: *C1CCN(*)CC1 ; *C1CCN(*)CC1 ; *N1CCN(*)CC1]

In one embodiment of compound of formula (IB), L³ is a bond or a group selected from —CH₂—, —CH₂—CH₂—, —CH(CH₃)—CH₂—.

In one embodiment of compound of formula (IB), ‘n’ is an integer selected from 0, 1 and 2.

In one embodiment of compound of formula (IB), L³ is selected from

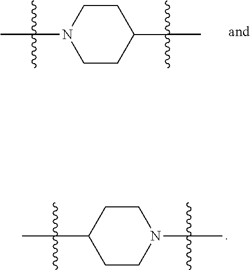 [SMILES: *C1CCN(*)CC1 ; *C1CCN(*)CC1]

In one embodiment of compound of formula (IB), the group

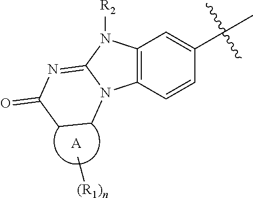

represents

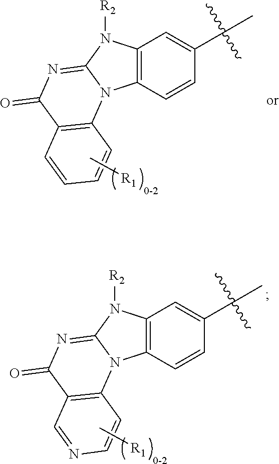

wherein each R₁ is halogen, (C₁-C₆)alkyl, (C₁-C₆)alkoxy, amino or hydroxy; and each R₂ is hydrogen, halogen, hydroxy, (C₁-C₆)alkyl or 3- to 8-membered cycloalkyl.

In one embodiment of compound of formula (IB), the group

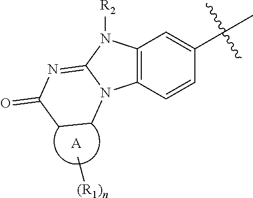

represents

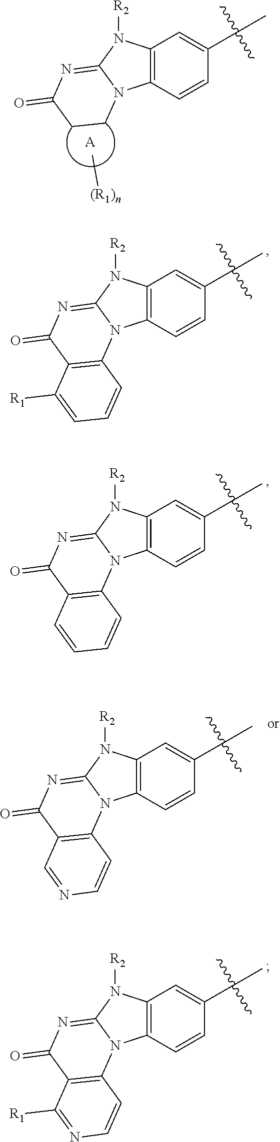

wherein each R₁ is selected from —Cl, —Br, —CH₃, —CF₂, —CF₃, —O—CH₃, —O—CH₂—CH₃, —O—CH(CH₃)—CH₃, —OH and —NH₂; and each R₂ is hydrogen, —CH₃, —CH(CH₃)₂, cyclopropyl, cyclobutyl or cyclopentyl.

In one embodiment of compound of formula (IB), Y₁ is CH or N.

In one embodiment of compound of formula (IB), Y₁ is N.

In one embodiment of compound of formula (IB), Y₂ is CH.

In one embodiment of compound of formula (IB), Q represents

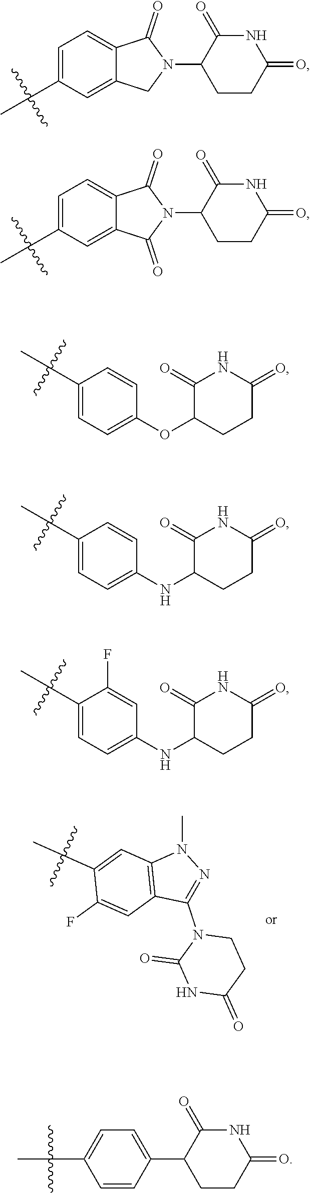

In one embodiment, the present invention provides compound of formula (IB) or a pharmaceutically acceptable salt or a stereoisomer or a tautomer thereof, wherein
A is phenyl or pyridyl;
each R₁ is halogen, (C₁-C₆)alkyl, (C₁-C₆)alkoxy, halo(C₁-C₆)alkyl, amino or hydroxy;
Y₁ and Y₂ independently is CH or N;
R₂ is hydrogen, halogen, hydroxy, (C₁-C₆)alkyl or 3- to 8-membered cycloalkyl;
L₁ is a bond or a group selected from

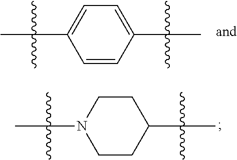

L₃ is a bond or a group selected from —CH₂—, —CH₂—CH₂—, —CH(CH₃)—CH₂—,

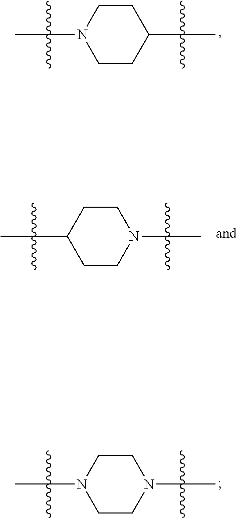 [SMILES: *C1CCN(*)CC1 ; *C1CCN(*)CC1 ; *N1CCN(*)CC1]

and

Q represents

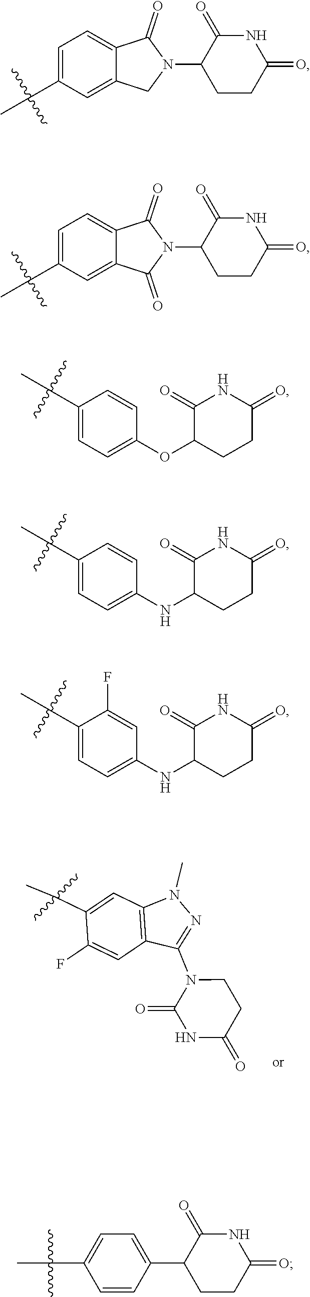

and
‘n’ is an integer selected from 0, 1 and 2.

In one embodiment, the present invention provides compound of formula (IC) or a pharmaceutically acceptable salt or a stereoisomer or a tautomer thereof:

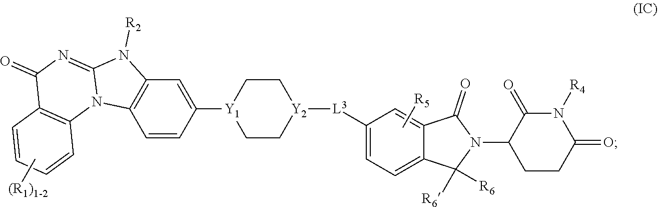

wherein Y₁ and Y₂ independently is CH or N;
L³ is (C₁-C₆)alkylenyl or 6-membered heterocycloalkylenyl; and
R₁, R₂, R₄, R₅, R₆ and R₆′ are as defined in compound of formula (I).
In one embodiment of compound of formula (IC), Y₁ and Y₂ independently is CH or N.

In one embodiment of compound of formula (IC), Y₂ is CH or N.

In one embodiment of compound of formula (IC), Y₁ is N.

In one embodiment of compound of formula (IC), R₂ is hydrogen, halogen, hydroxy, (C₁-C₆)alkyl or 3- to 8-membered cycloalkyl.

In one embodiment of compound of formula (IC), R₁ is selected from —Cl, —Br, —CH₃, —CF₂, —CF₃, —O—CH₃, —O—CH₂—CH₃, —O—CH(CH₃)—CH₃, —OH and —NH₂.

In one embodiment of compound of formula (IC), R₁ is selected from —Br, —Cl, —NH₂, —CH₃, —OCH₃, —OH and —CF₃.

In one embodiment of compound of formula (IC), R₂ is hydrogen, —CH₃, —CH(CH₃)₂, cyclopropyl, cyclobutyl or cyclopentyl.

In one embodiment of compound of formula (IC), L³ is a bond or a group selected from —CH₂—, —CH₂—CH₂—, —CH(CH₃)—CH₂—,

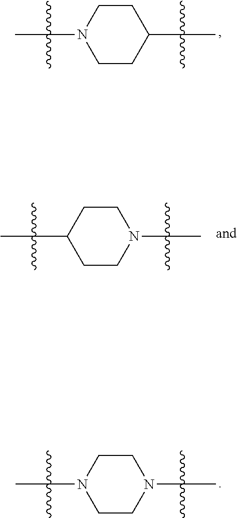 [SMILES: *C1CCN(*)CC1 ; *C1CCN(*)CC1 ; *N1CCN(*)CC1]

In one embodiment of compound of formula (IC), L³ is a bond.

In one embodiment of compound of formula (IC), L³ is selected from —CH₂—, —CH₂—CH₂—,

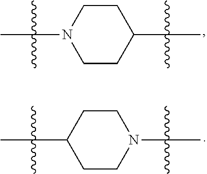 [SMILES: *C1CCN(*)CC1 ; *C1CCN(*)CC1]

In one embodiment of compound of formula (IC), the group

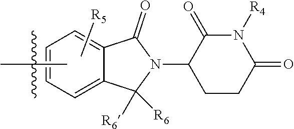

represents

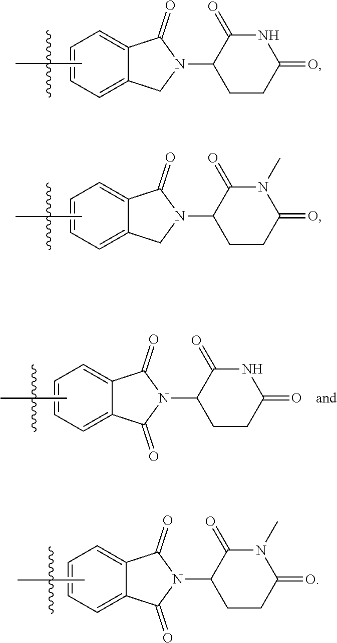

In one embodiment of compound of formula (IC), the group

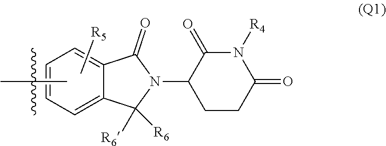

represents

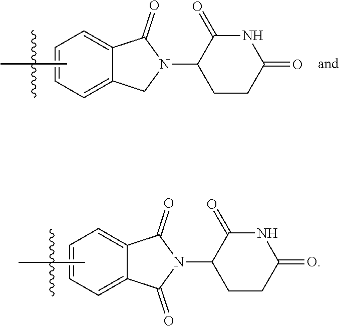

In one embodiment, the present invention provides compound of formula (IC) or a pharmaceutically acceptable salt or a stereoisomer or a tautomer thereof: wherein
Y₁ and Y₂ independently is CH or N;
L³ is selected from —CH₂, —CH₂CH₂—,

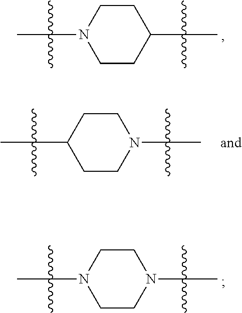 [SMILES: *C1CCN(*)CC1 ; *C1CCN(*)CC1 ; *N1CCN(*)CC1]

R₁ is selected from —Br, —Cl, —NH₂, —CH₃, —OCH₃, —OH and —CF₃;
R₂ is hydrogen, —CH₃, —CH(CH₃)₂, cyclopropyl, cyclobutyl or cyclopentyl; and

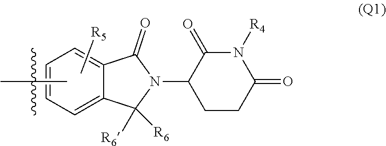

represents

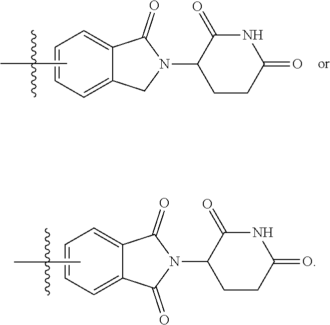

In one embodiment, the present invention provides compound of formula (ID) or a pharmaceutically acceptable salt or a stereoisomer or a tautomer thereof:

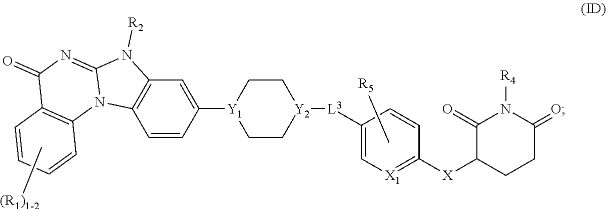

wherein Y₁ and Y₂ independently is CH or N;
L³ is (C₁-C₆)alkylenyl or 6-membered heterocycloalkylenyl; and
R₁, R₂, R₄, R₅, X and X₁ are as defined in compound of formula (I).

In one embodiment of compound of formula (ID), Y₁ and Y₂ independently is CH or N.

In one embodiment of compound of formula (ID), Y₂ is CH or N.

In one embodiment of compound of formula (ID), Y₁ is N.

In one embodiment of compound of formula (ID), R₂ is hydrogen, halogen, hydroxy, (C₁-C₆)alkyl or 3- to 8-membered cycloalkyl.

In one embodiment of compound of formula (ID), R₁ is selected from —Cl, —Br, —CH₃, —CF₂, —CF₃, —O—CH₃, —O—CH₂—CH₃, —O—CH(CH₃)—CH₃, —OH and —NH₂.

In one embodiment of compound of formula (ID), R₁ is selected from —Br, —Cl, —NH₂, —CH₃, —OCH₃, —OH and —CF₃.

In one embodiment of compound of formula (ID), R₂ is hydrogen, —CH₃, —CH(CH₃)₂, cyclopropyl, cyclobutyl or cyclopentyl.

In one embodiment of compound of formula (ID), L³ is a bond or a group selected from —CH₂—, —CH₂—CH₂—, —CH(CH₃)—CH₂—,

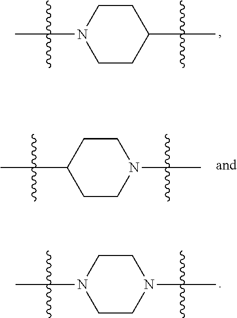 [SMILES: *C1CCN(*)CC1 ; *C1CCN(*)CC1 ; *N1CCN(*)CC1]

In one embodiment of compound of formula (ID), L³ is a bond.

In one embodiment of compound of formula (ID), L³ is selected from —CH₂—, —CH₂—CH₂—,

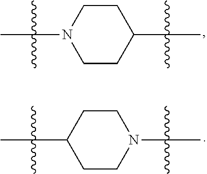 [SMILES: *C1CCN(*)CC1 ; *C1CCN(*)CC1]

In one embodiment of compound of formula (ID), L³ is selected from

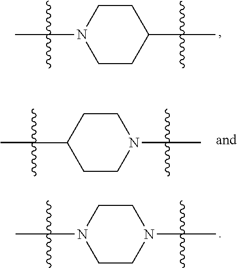

In one embodiment of compound of formula (ID),

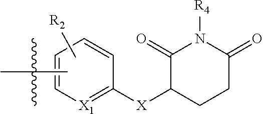

represents

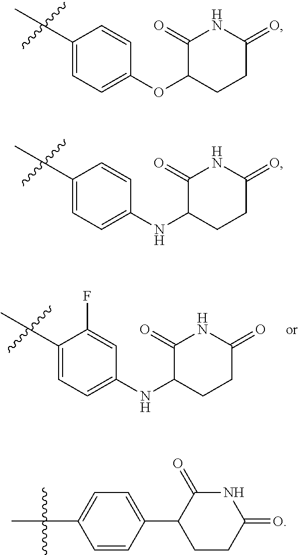 [SMILES: *c1ccc(OC2CCC(=O)NC2=O)cc1 ; *c1ccc(NC2CCC(=O)NC2=O)cc1 ; *c1ccc(NC2CCC(=O)NC2=O)cc1F ; *c1ccc(C2CCC(=O)NC2=O)cc1]

In one embodiment of compound of formula (ID),

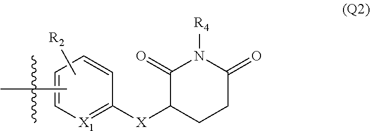

represents

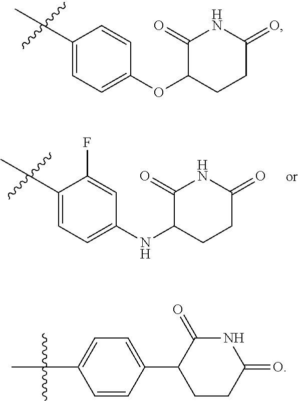 [SMILES: *c1ccc(OC2CCC(=O)NC2=O)cc1 ; *c1ccc(NC2CCC(=O)NC2=O)cc1F ; *c1ccc(C2CCC(=O)NC2=O)cc1]

In one embodiment, the present invention provides compound of formula (ID) or a pharmaceutically acceptable salt or a stereoisomer or a tautomer thereof: wherein
Y₁ and Y₂ independently is CH or N;
L³ is selected from —CH₂, —CH₂CH₂—,

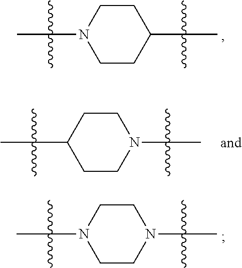

R₁ is selected from —Br, —Cl, —NH₂, —CH₃, —OCH₃, —OH and —CF₃;

R₂ is hydrogen, —CH₃, —CH(CH₃)₂, cyclopropyl, cyclobutyl or cyclopentyl; and

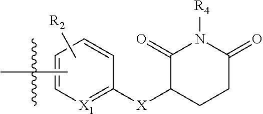

represents

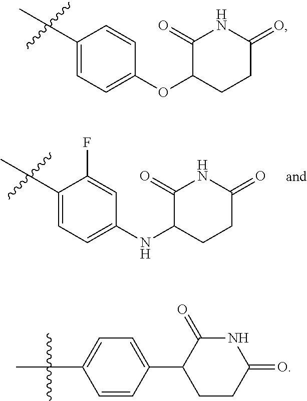 [SMILES: *c1ccc(OC2CCC(=O)NC2=O)cc1 ; *c1ccc(NC2CCC(=O)NC2=O)cc1F ; *c1ccc(C2CCC(=O)NC2=O)cc1]

In one embodiment, the present invention provides a compound of formula (I) selected from the ones described herein, or a pharmaceutically acceptable salt or a stereoisomer or a tautomer thereof.

In one embodiment the present invention provides the compound of formula (IE), (IF) and (IG);

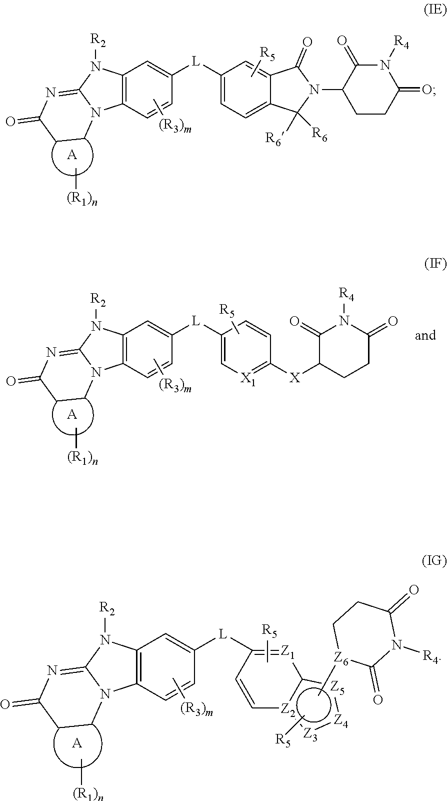

In one embodiment, the present invention provides compound of formula (I),

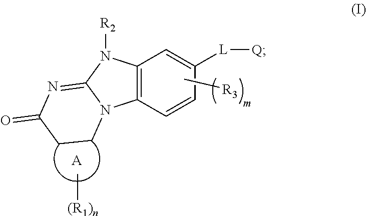

or a pharmaceutically acceptable salt or a stereoisomer or a tautomer thereof;
wherein,
   A is phenyl or pyridyl;
   each R₁ and R₃ independently is halogen, (C₁-C₆)alkyl, (C₁-C₆)alkoxy, amino, halo(C₁-C₆)alkyl, hydroxy, 3- to 8-membered cycloalkyl, hydroxy (C₁-C₆)alkyl or cyano;
   R₂ is hydrogen, halogen, hydroxy, (C₁-C₆)alkyl or 3- to 8-membered cycloalkyl; wherein the alkyl and cycloalkyl is substituted with 0, 1, 2 or 3 substituents, independently, selected from hydroxy, halogen and alkoxy;
   L is

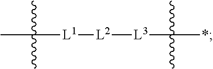

wherein asterisk mark represents the point of attachment with Q;
L¹ and L³ independently is a bond, (C₁-C₆)alkylenyl, 6- to 10-membered arylenyl or 5- to 6-membered heterocycloalkylenyl; wherein the alkylenyl, arylenyl and heterocycloalkylenyl, at each occurrence, independently is substituted with 0, 1, 2, 3 or 4 R₇;
L² is 5- to 6-membered heterocycloalkylenyl which is substituted with 0, 1, 2, 3 or 4 R₇;
each R₇ is halogen, hydroxy, (C₁-C₆)alkyl, (C₁-C₆)alkoxy or halo(C₁-C₆)alkyl;
Q is represented by formula Q1 or Q2:

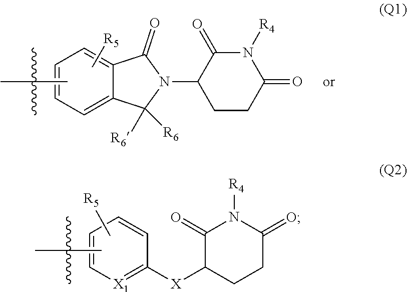

wherein,
   X is a bond, —O— or —NH—;
   X₁ is N or CH;
   each R₅ is hydrogen, halogen, halo(C₁-C₆)alkyl or (C₁-C₆)alkyl;
   R₆ and R₆′ independently is hydrogen; or R₆ and R₆′ together represent an oxo group; and
   each R₄ is hydrogen or (C₁-C₆)alkyl; and
   ‘n’ and ‘m’ are an integer, independently selected from 0, 1, 2 and 3.

In one embodiment of the present invention, the compound is selected from;

| Compound | |
|---|---|
| No. | Structure |
| 1 | 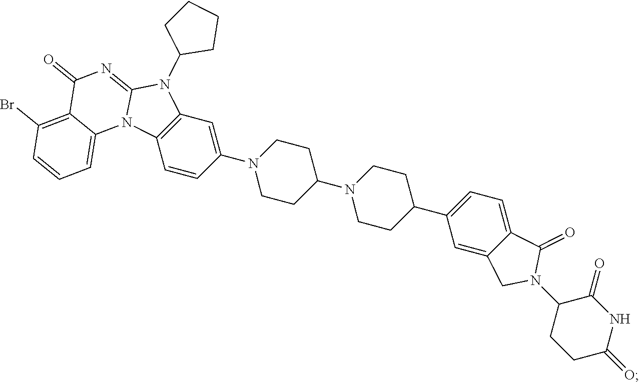 [SMILES: O=C1CCC(N2Cc3cc(C4CCN(C5CCN(c6ccc7c(c6)n(C6CCCC6)c6nc(=O)c8c(Br)cccc8n76)CC5)CC4)ccc3C2=O)C(=O)N1] |
| 2 | 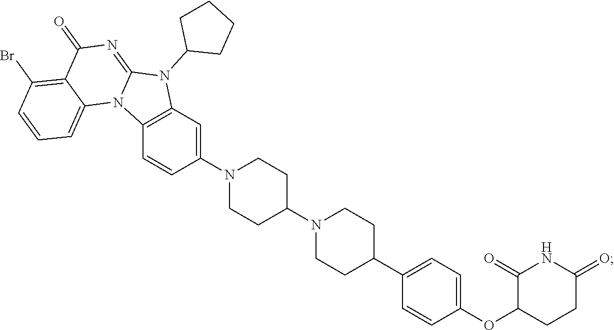 [SMILES: O=C1CCC(Oc2ccc(C3CCN(C4CCN(c5ccc6c(c5)n(C5CCCC5)c5nc(=O)c7c(Br)cccc7n65)CC4)CC3)cc2)C(=O)N1] |
| 3 | 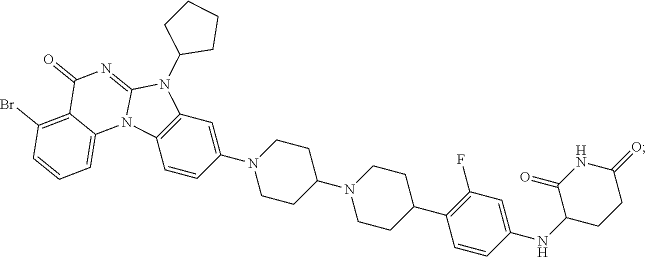 [SMILES: O=C1CCC(Nc2ccc(C3CCN(C4CCN(c5ccc6c(c5)n(C5CCCC5)c5nc(=O)c7c(Br)cccc7n65)CC4)CC3)c(F)c2)C(=O)N1] |
| 4 | 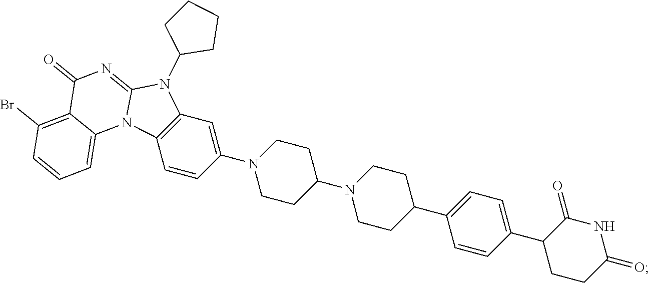 [SMILES: O=C1CCC(c2ccc(C3CCN(C4CCN(c5ccc6c(c5)n(C5CCCC5)c5nc(=O)c7c(Br)cccc7n65)CC4)CC3)cc2)C(=O)N1] |
| 5 | 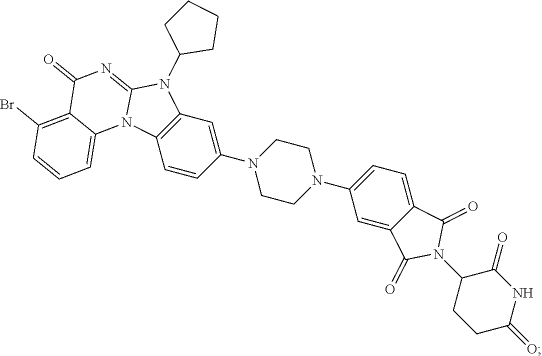 [SMILES: O=C1CCC(N2C(=O)c3ccc(N4CCN(c5ccc6c(c5)n(C5CCCC5)c5nc(=O)c7c(Br)cccc7n65)CC4)cc3C2=O)C(=O)N1] |
| 6 | 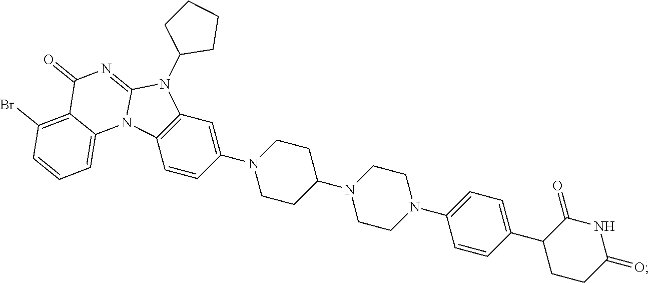 [SMILES: O=C1CCC(c2ccc(N3CCN(C4CCN(c5ccc6c(c5)n(C5CCCC5)c5nc(=O)c7c(Br)cccc7n65)CC4)CC3)cc2)C(=O)N1] |
| 7 | 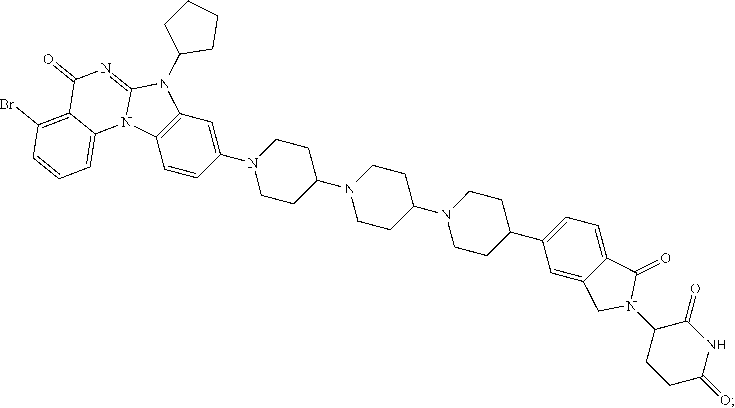 [SMILES: O=C1CCC(N2Cc3cc(C4CCN(C5CCN(C6CCN(c7ccc8c(c7)n(C7CCCC7)c7nc(=O)c9c(Br)cccc9n87)CC6)CC5)CC4)ccc3C2=O)C(=O)N1] |
| 8 | 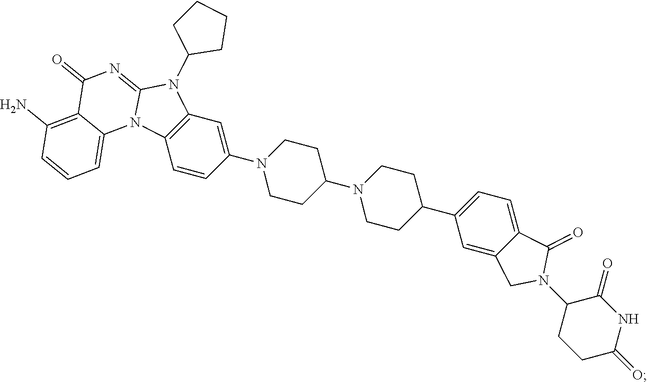 [SMILES: Nc1cccc2c1c(=O)nc1n(C3CCCC3)c3cc(N4CCC(N5CCC(c6ccc7c(c6)CN(C6CCC(=O)NC6=O)C7=O)CC5)CC4)ccc3n21] |
| 9 | 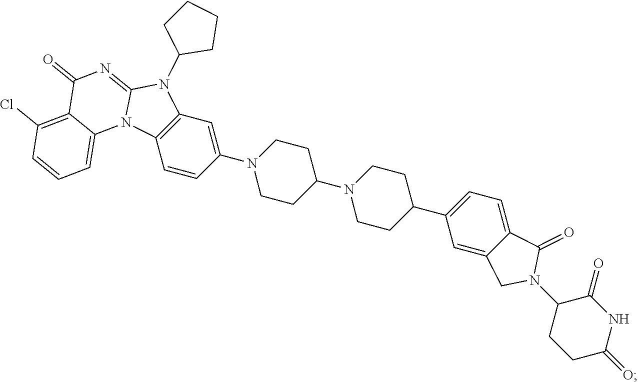 [SMILES: O=C1CCC(N2Cc3cc(C4CCN(C5CCN(c6ccc7c(c6)n(C6CCCC6)c6nc(=O)c8c(Cl)cccc8n76)CC5)CC4)ccc3C2=O)C(=O)N1] |
| 10 | 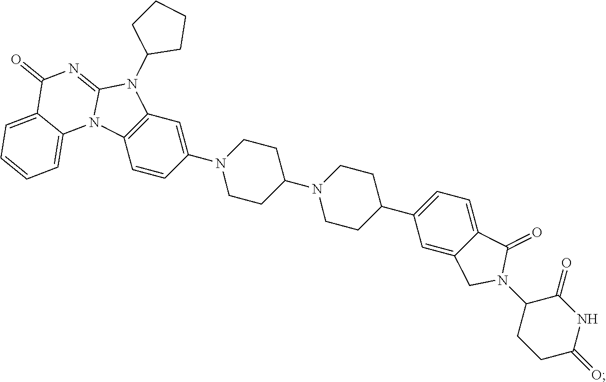 [SMILES: O=C1CCC(N2Cc3cc(C4CCN(C5CCN(c6ccc7c(c6)n(C6CCCC6)c6nc(=O)c8ccccc8n76)CC5)CC4)ccc3C2=O)C(=O)N1] |
| 11 | 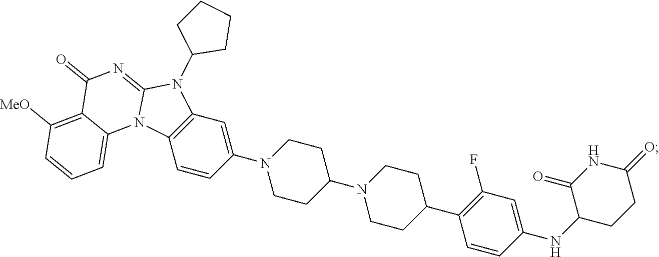 [SMILES: COc1cccc2c1c(=O)nc1n(C3CCCC3)c3cc(N4CCC(N5CCC(c6ccc(NC7CCC(=O)NC7=O)cc6F)CC5)CC4)ccc3n21] |
| 12 | 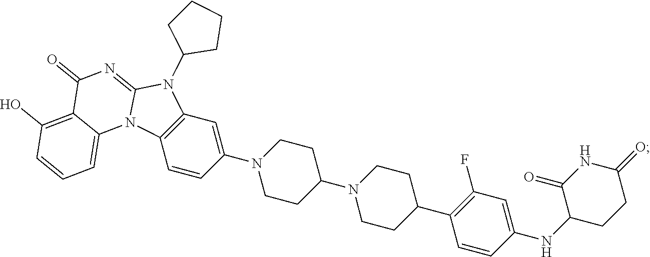 [SMILES: O=C1CCC(Nc2ccc(C3CCN(C4CCN(c5ccc6c(c5)n(C5CCCC5)c5nc(=O)c7c(O)cccc7n65)CC4)CC3)c(F)c2)C(=O)N1] |
| 13 | 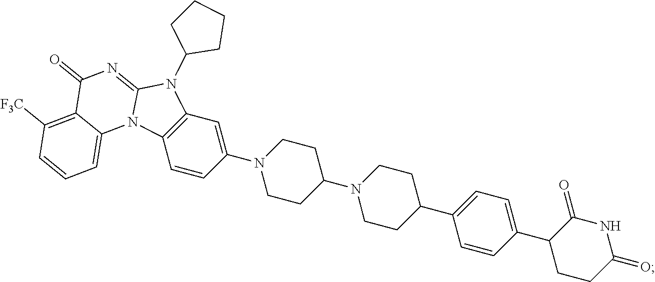 [SMILES: O=C1CCC(c2ccc(C3CCN(C4CCN(c5ccc6c(c5)n(C5CCCC5)c5nc(=O)c7c(C(F)(F)F)cccc7n65)CC4)CC3)cc2)C(=O)N1] |
| 14 | 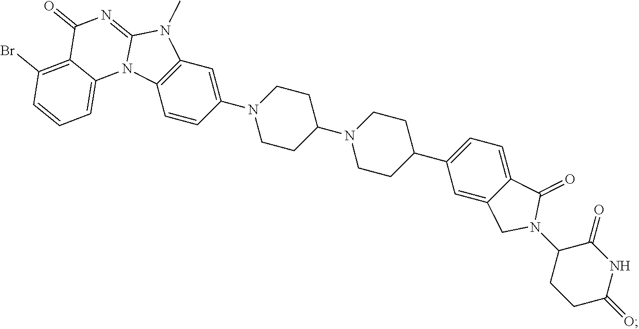 [SMILES: Cn1c2cc(N3CCC(N4CCC(c5ccc6c(c5)CN(C5CCC(=O)NC5=O)C6=O)CC4)CC3)ccc2n2c3cccc(Br)c3c(=O)nc12] |
| 15 | 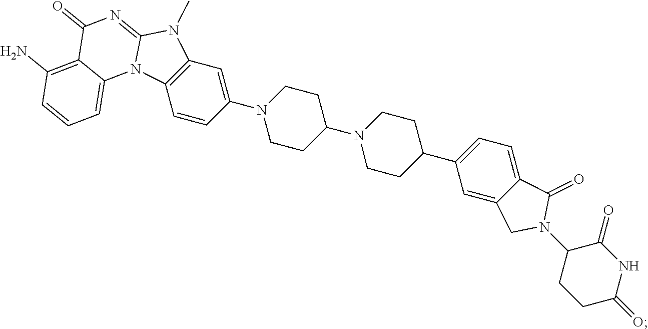 [SMILES: Cn1c2cc(N3CCC(N4CCC(c5ccc6c(c5)CN(C5CCC(=O)NC5=O)C6=O)CC4)CC3)ccc2n2c3cccc(N)c3c(=O)nc12] |
| 16 | 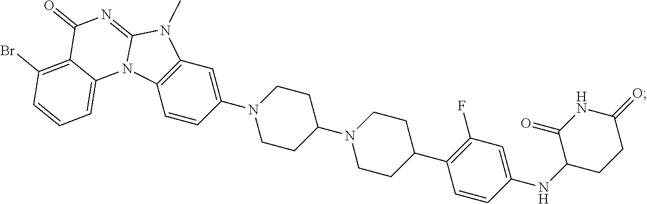 [SMILES: Cn1c2cc(N3CCC(N4CCC(c5ccc(NC6CCC(=O)NC6=O)cc5F)CC4)CC3)ccc2n2c3cccc(Br)c3c(=O)nc12] |
| 17 | 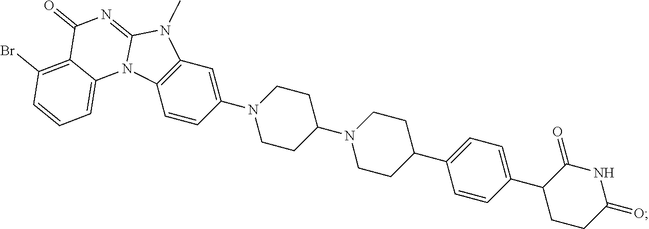 [SMILES: Cn1c2cc(N3CCC(N4CCC(c5ccc(C6CCC(=O)NC6=O)cc5)CC4)CC3)ccc2n2c3cccc(Br)c3c(=O)nc12] |
| 18 | 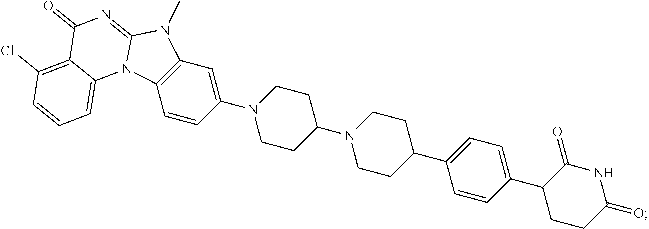 [SMILES: Cn1c2cc(N3CCC(N4CCC(c5ccc(C6CCC(=O)NC6=O)cc5)CC4)CC3)ccc2n2c3cccc(Cl)c3c(=O)nc12] |
| 19 | 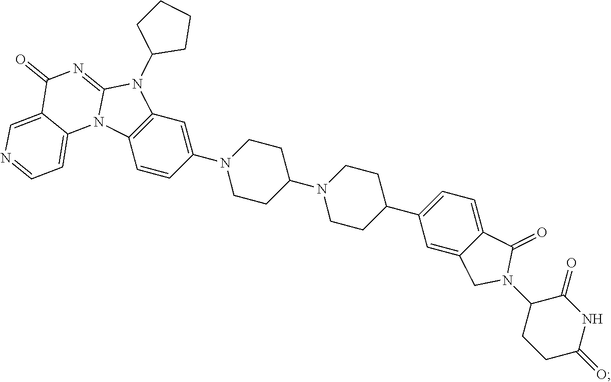 [SMILES: O=C1CCC(N2Cc3cc(C4CCN(C5CCN(c6ccc7c(c6)n(C6CCCC6)c6nc(=O)c8cnccc8n76)CC5)CC4)ccc3C2=O)C(=O)N1] |
| 20 | 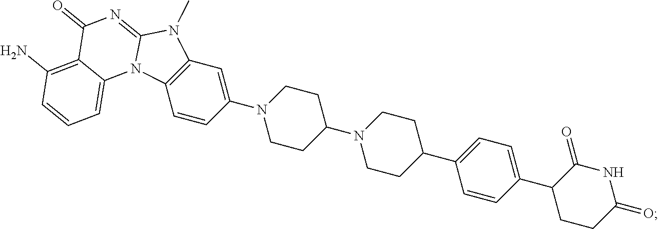 [SMILES: Cn1c2cc(N3CCC(N4CCC(c5ccc(C6CCC(=O)NC6=O)cc5)CC4)CC3)ccc2n2c3cccc(N)c3c(=O)nc12] |
| 21 | 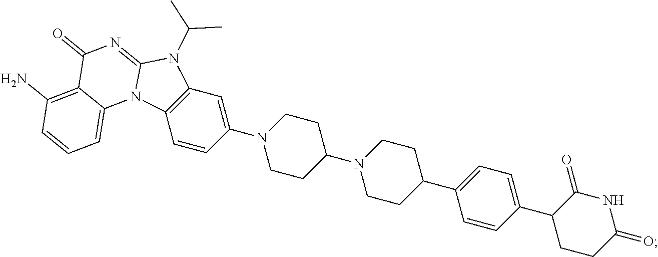 [SMILES: CC(C)n1c2cc(N3CCC(N4CCC(c5ccc(C6CCC(=O)NC6=O)cc5)CC4)CC3)ccc2n2c3cccc(N)c3c(=O)nc12] |
| 22 | 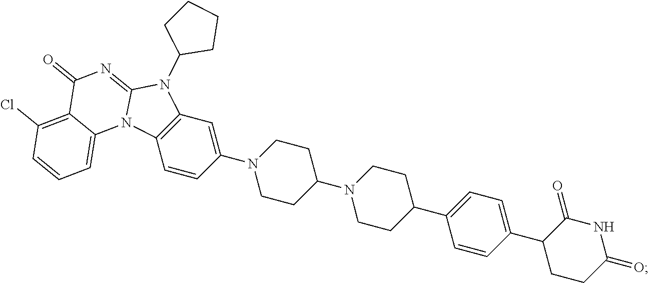 [SMILES: O=C1CCC(c2ccc(C3CCN(C4CCN(c5ccc6c(c5)n(C5CCCC5)c5nc(=O)c7c(Cl)cccc7n65)CC4)CC3)cc2)C(=O)N1] |
| 23 | 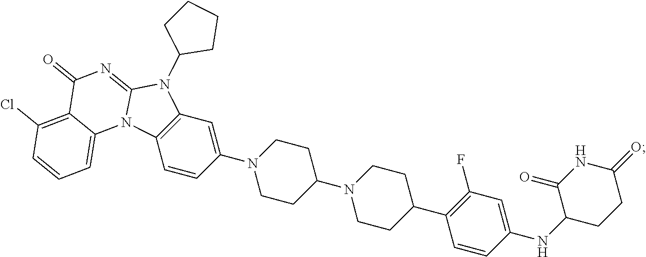 [SMILES: O=C1CCC(Nc2ccc(C3CCN(C4CCN(c5ccc6c(c5)n(C5CCCC5)c5nc(=O)c7c(Cl)cccc7n65)CC4)CC3)c(F)c2)C(=O)N1] |
| 24 | 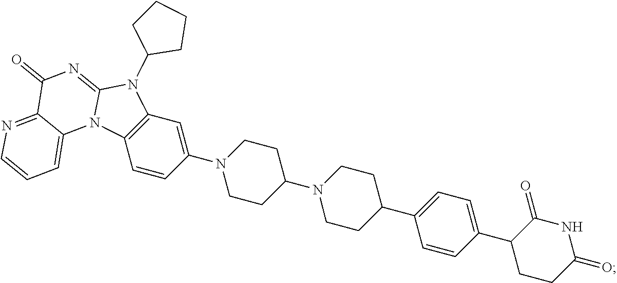 [SMILES: O=C1CCC(c2ccc(C3CCN(C4CCN(c5ccc6c(c5)n(C5CCCC5)c5nc(=O)c7ncccc7n65)CC4)CC3)cc2)C(=O)N1] |
| 25 | 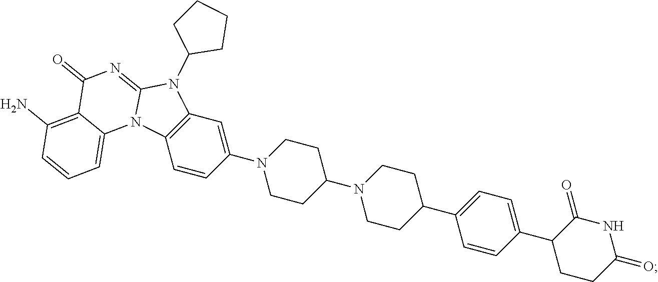 [SMILES: Nc1cccc2c1c(=O)nc1n(C3CCCC3)c3cc(N4CCC(N5CCC(c6ccc(C7CCC(=O)NC7=O)cc6)CC5)CC4)ccc3n21] |
| 26 | 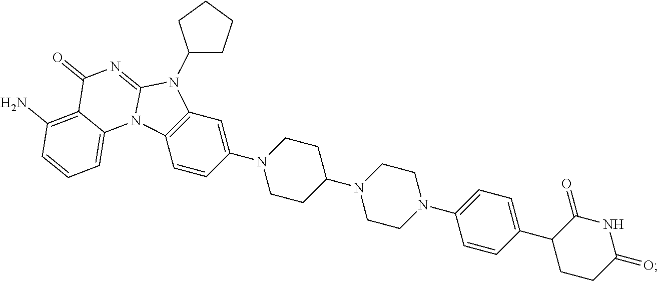 [SMILES: Nc1cccc2c1c(=O)nc1n(C3CCCC3)c3cc(N4CCC(N5CCN(c6ccc(C7CCC(=O)NC7=O)cc6)CC5)CC4)ccc3n21] |
| 27 | 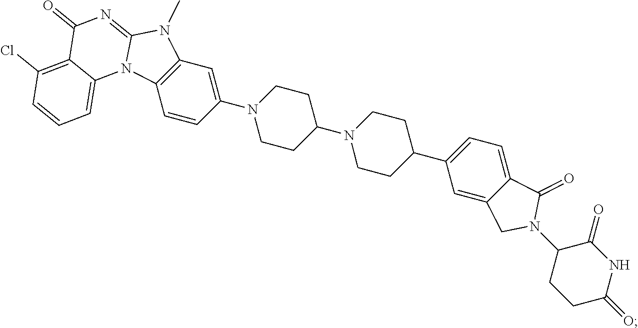 [SMILES: Cn1c2cc(N3CCC(N4CCC(c5ccc6c(c5)CN(C5CCC(=O)NC5=O)C6=O)CC4)CC3)ccc2n2c3cccc(Cl)c3c(=O)nc12] |
| 28 | 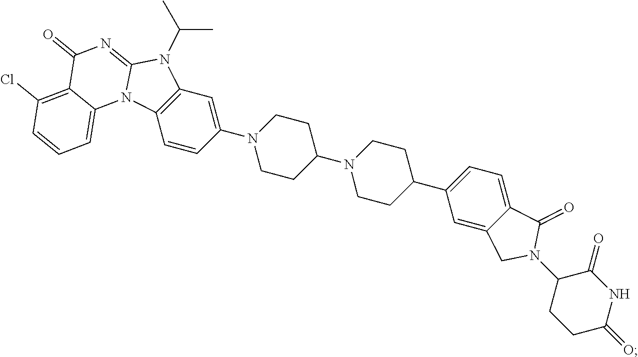 [SMILES: CC(C)n1c2cc(N3CCC(N4CCC(c5ccc6c(c5)CN(C5CCC(=O)NC5=O)C6=O)CC4)CC3)ccc2n2c3cccc(Cl)c3c(=O)nc12] |
| 29 | 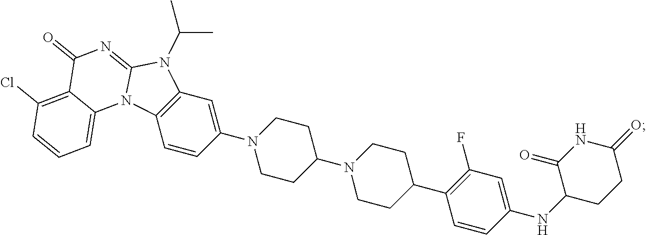 [SMILES: CC(C)n1c2cc(N3CCC(N4CCC(c5ccc(NC6CCC(=O)NC6=O)cc5F)CC4)CC3)ccc2n2c3cccc(Cl)c3c(=O)nc12] |
| 30 | 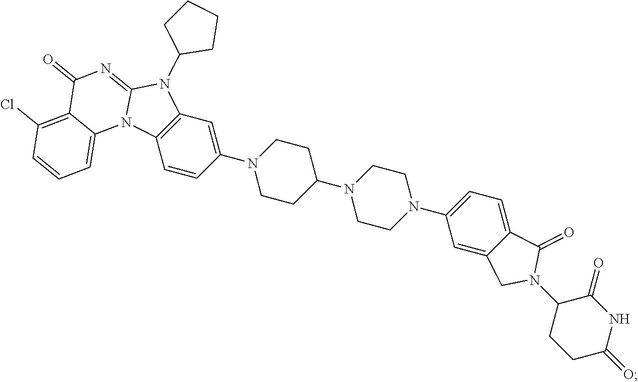 [SMILES: O=C1CCC(N2Cc3cc(N4CCN(C5CCN(c6ccc7c(c6)n(C6CCCC6)c6nc(=O)c8c(Cl)cccc8n76)CC5)CC4)ccc3C2=O)C(=O)N1] |
| 31 | 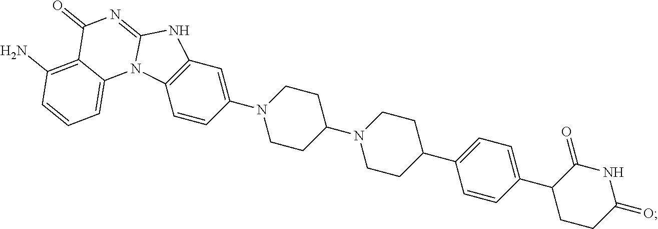 [SMILES: Nc1cccc2c1c(=O)nc1[nH]c3cc(N4CCC(N5CCC(c6ccc(C7CCC(=O)NC7=O)cc6)CC5)CC4)ccc3n12] |
| 32 | 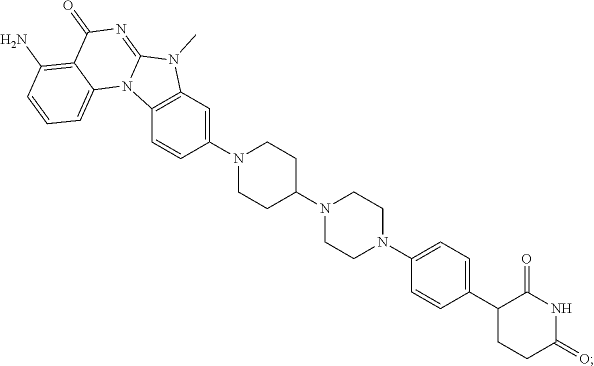 [SMILES: Cn1c2cc(N3CCC(N4CCN(c5ccc(C6CCC(=O)NC6=O)cc5)CC4)CC3)ccc2n2c3cccc(N)c3c(=O)nc12] |
| 33 | 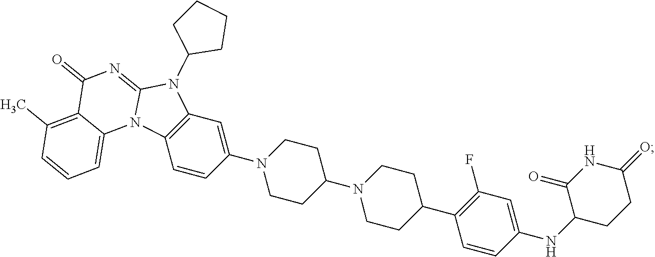 [SMILES: Cc1cccc2c1c(=O)nc1n(C3CCCC3)c3cc(N4CCC(N5CCC(c6ccc(NC7CCC(=O)NC7=O)cc6F)CC5)CC4)ccc3n21] |
| 34 | 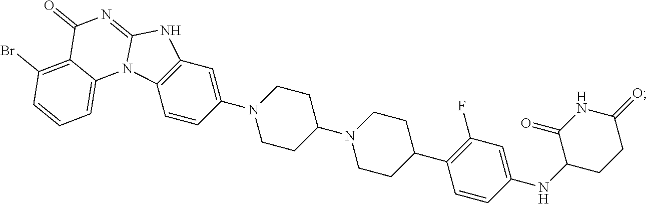 [SMILES: O=C1CCC(Nc2ccc(C3CCN(C4CCN(c5ccc6c(c5)[nH]c5nc(=O)c7c(Br)cccc7n56)CC4)CC3)c(F)c2)C(=O)N1] |
| 35 | 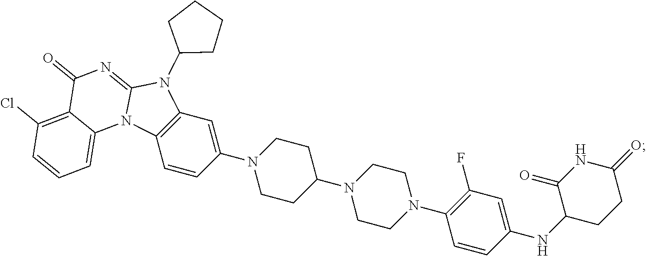 [SMILES: O=C1CCC(Nc2ccc(N3CCN(C4CCN(c5ccc6c(c5)n(C5CCCC5)c5nc(=O)c7c(Cl)cccc7n65)CC4)CC3)c(F)c2)C(=O)N1] |
| 36 | 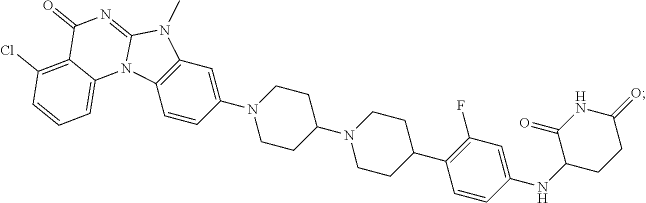 [SMILES: Cn1c2cc(N3CCC(N4CCC(c5ccc(NC6CCC(=O)NC6=O)cc5F)CC4)CC3)ccc2n2c3cccc(Cl)c3c(=O)nc12] |
| 37 | 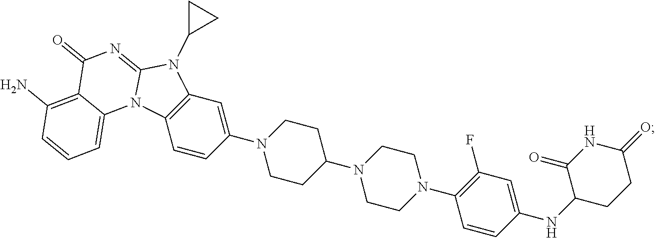 [SMILES: Nc1cccc2c1c(=O)nc1n(C3CC3)c3cc(N4CCC(N5CCN(c6ccc(NC7CCC(=O)NC7=O)cc6F)CC5)CC4)ccc3n21] |
| 38 | 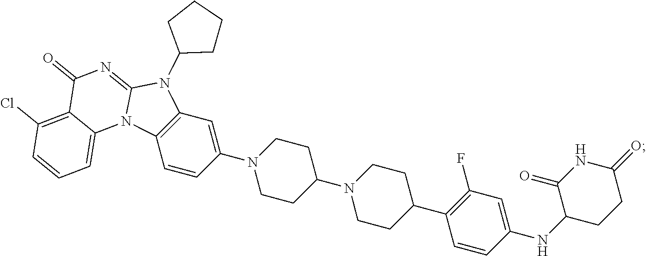 [SMILES: O=C1CCC(Nc2ccc(C3CCN(C4CCN(c5ccc6c(c5)n(C5CCCC5)c5nc(=O)c7c(Cl)cccc7n65)CC4)CC3)c(F)c2)C(=O)N1] |
| 39 | 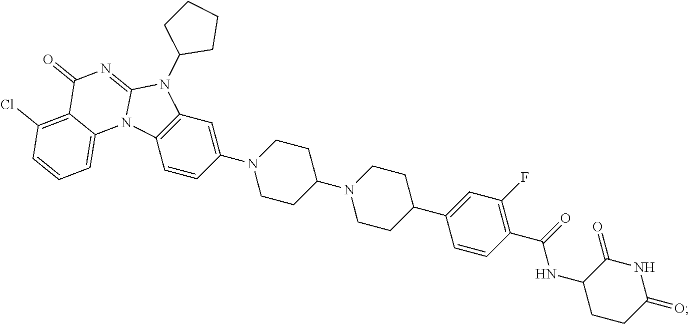 [SMILES: O=C1CCC(NC(=O)c2ccc(C3CCN(C4CCN(c5ccc6c(c5)n(C5CCCC5)c5nc(=O)c7c(Cl)cccc7n65)CC4)CC3)cc2F)C(=O)N1] |
| 40 | 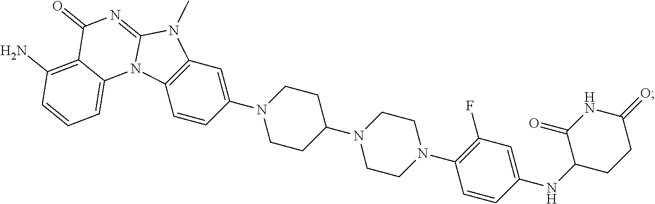 [SMILES: Cn1c2cc(N3CCC(N4CCN(c5ccc(NC6CCC(=O)NC6=O)cc5F)CC4)CC3)ccc2n2c3cccc(N)c3c(=O)nc12] |
| 41 | 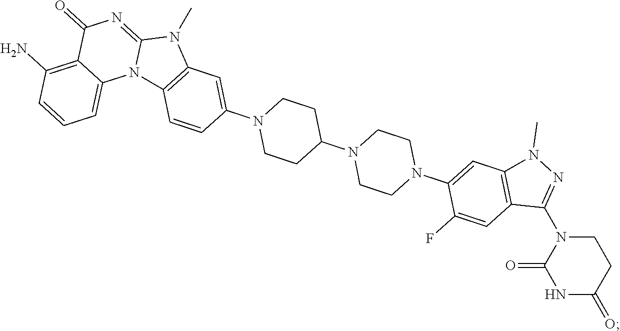 [SMILES: Cn1nc(N2CCC(=O)NC2=O)c2cc(F)c(N3CCN(C4CCN(c5ccc6c(c5)n(C)c5nc(=O)c7c(N)cccc7n65)CC4)CC3)cc21] |
| 42 | 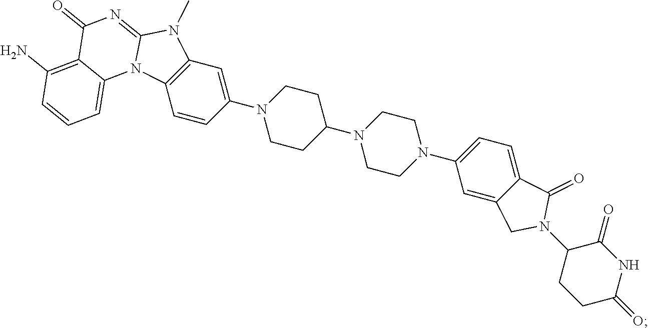 [SMILES: Cn1c2cc(N3CCC(N4CCN(c5ccc6c(c5)CN(C5CCC(=O)NC5=O)C6=O)CC4)CC3)ccc2n2c3cccc(N)c3c(=O)nc12] |
| 43 | 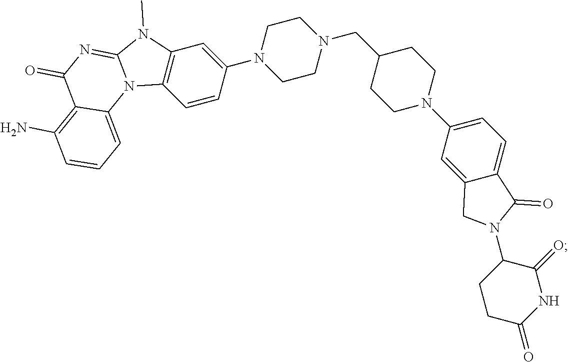 [SMILES: Cn1c2cc(N3CCN(CC4CCN(c5ccc6c(c5)CN(C5CCC(=O)NC5=O)C6=O)CC4)CC3)ccc2n2c3cccc(N)c3c(=O)nc12] |
| 44 | 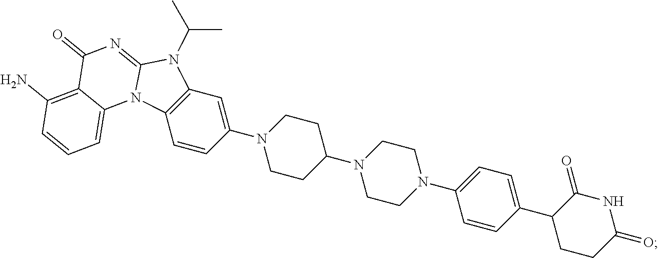 [SMILES: CC(C)n1c2cc(N3CCC(N4CCN(c5ccc(C6CCC(=O)NC6=O)cc5)CC4)CC3)ccc2n2c3cccc(N)c3c(=O)nc12] |
| 45 | 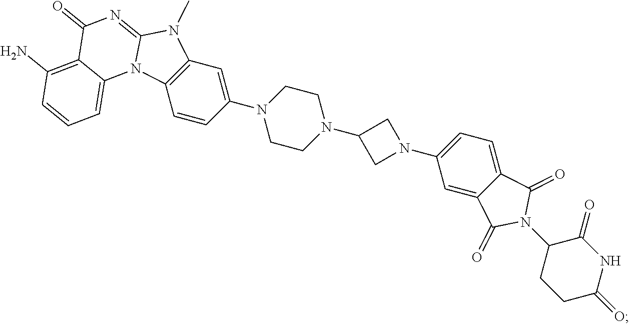 [SMILES: Cn1c2cc(N3CCN(C4CN(c5ccc6c(c5)C(=O)N(C5CCC(=O)NC5=O)C6=O)C4)CC3)ccc2n2c3cccc(N)c3c(=O)nc12] |
| 46 | 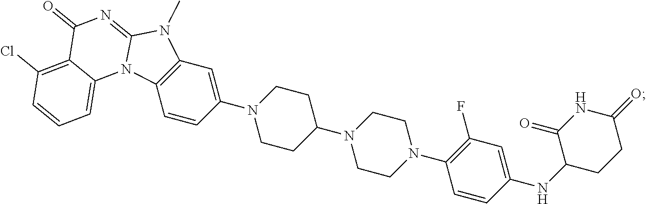 [SMILES: Cn1c2cc(N3CCC(N4CCN(c5ccc(NC6CCC(=O)NC6=O)cc5F)CC4)CC3)ccc2n2c3cccc(Cl)c3c(=O)nc12] |
| 47 | 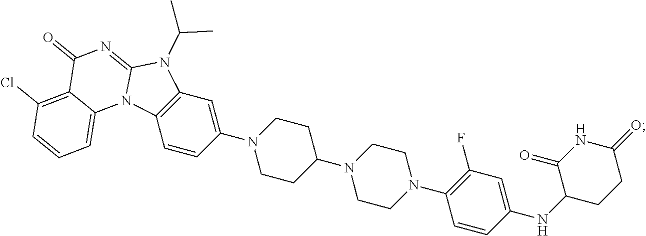 [SMILES: CC(C)n1c2cc(N3CCC(N4CCN(c5ccc(NC6CCC(=O)NC6=O)cc5F)CC4)CC3)ccc2n2c3cccc(Cl)c3c(=O)nc12] |
| 48 | 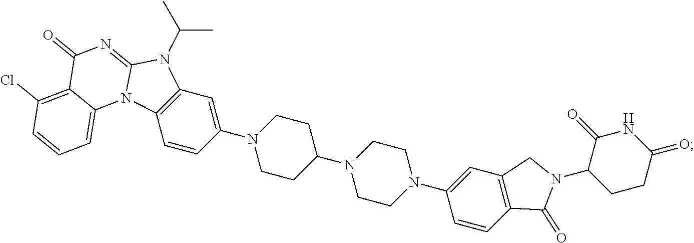 [SMILES: CC(C)n1c2cc(N3CCC(N4CCN(c5ccc6c(c5)CN(C5CCC(=O)NC5=O)C6=O)CC4)CC3)ccc2n2c3cccc(Cl)c3c(=O)nc12] |
| 49 | 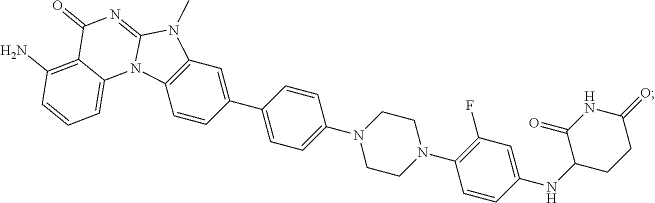 [SMILES: Cn1c2cc(-c3ccc(N4CCN(c5ccc(NC6CCC(=O)NC6=O)cc5F)CC4)cc3)ccc2n2c3cccc(N)c3c(=O)nc12] |
| 50 | 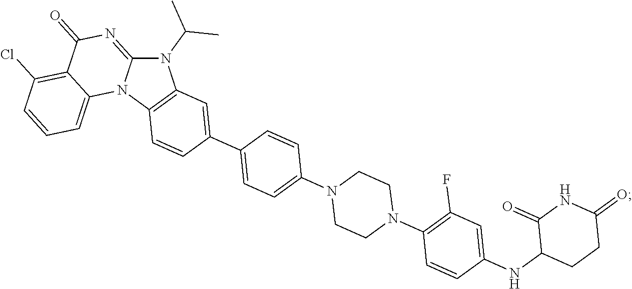 [SMILES: CC(C)n1c2cc(-c3ccc(N4CCN(c5ccc(NC6CCC(=O)NC6=O)cc5F)CC4)cc3)ccc2n2c3cccc(Cl)c3c(=O)nc12] |

or a pharmaceutically acceptable salt or a stereoisomer or a tautomer thereof.

### Pharmaceutical Compositions

In one embodiment, the present application provides a pharmaceutical composition comprising a compound of formula (I), or a pharmaceutically acceptable salt or a stereoisomer thereof, and at least one pharmaceutically acceptable carrier or pharmaceutically acceptable excipients.

Illustrative pharmaceutical compositions are tablets and gelatin capsules comprising one or more compounds of the present disclosure and a pharmaceutically acceptable carrier, such as, but not limited to, a) a diluent, e.g., purified water, triglyceride oils, such as hydrogenated or partially hydrogenated vegetable oil or mixtures thereof, corn oil, olive oil, sunflower oil, safflower oil, fish oils, such as EPA or DHA or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminium silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, alginic acid or its sodium salt or effervescent mixtures; e) absorbent, colorant, flavourant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

In one embodiment, the present invention provides a pharmaceutical composition for use in degrading a target protein in a subject, wherein the target protein is SMARCA2 and/or SMARCA4.

In one embodiment, the present invention provides a pharmaceutical composition wherein the subject is afflicted with a disease or disorder dependent upon SMARCA2 and/or SMARCA4.

In one embodiment, the present invention provides a pharmaceutical composition comprising a compound of formula (I), or a pharmaceutically acceptable salt or a stereoisomer thereof, for use in treating or preventing cancers selected from the group consisting of hematologic cancers, lung cancer, non-small cell lung cancer, acoustic neuroma, acute leukemia, acute lymphocytic leukemia, acute myelocytic leukemia, adenocarcinoma, angiosarcoma, astrocytoma, myelomonocytic and promyelocytic, acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic, granulocytic, leukemia, chronic myelogenous leukemia, colon cancer, colorectal cancer, craniopharyngioma, cystadenocarcinoma, diffuse large B-cell lymphoma, dysproliferative changes, dysplasias and metaplasias changes, embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, head and neck cancer, hemangioblastoma, hepatoma, hepatocellular cancer, hormone insensitive prostate cancer, leiomyosarcoma, leukemia, liposarcoma, liver cancer, lymphagioendotheliosarcoma, lymphangiosarcoma, lymphoblastic leukemia, lymphoma, Hodgkin's lymphoma, non-Hodgkin's lymphoma; Burkitt's lymphoma, malignancies and hyperproliferative disorders of the bladder, breast, colon, lung, ovaries, pancreas, prostate, skin and uterus, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, malignant rhabdoid tumor (MRT), rhabdomyosarcoma, sarcoma, sebaceous gland carcinoma, seminoma, skin cancer, small cell lung carcinoma, solid tumors, carcinomas, sarcomas, small cell lung cancer, stomach cancer, squamous cell carcinoma, synovioma, sweat gland carcinoma, thyroid cancer, Waldenstrom's macroglobulinemia, testicular tumors, uterine cancer or Wilms' tumor.

METHODS and/or USES:

In one embodiment, the present invention provides, a compound of formula (I), (IA), (IB), (IC), (ID), (IE), (IF) or (IG) or a pharmaceutically acceptable salt or a stereoisomer or a tautomer thereof, for use as a medicament.

In one embodiment, the present invention provides, a compound of formula (I), (IA), (IB), (IC), (ID), (IE), (IF) or (IG) or a pharmaceutical acceptable salt or a stereoisomer or a tautomer thereof, for use in the treatment of a disease or disorder dependent upon SMARCA2 and/or SMARCA4.

In one embodiment, a disease or disorder dependent upon SMARCA2 and/or SMARCA4 is cancer.

In one embodiment, the present invention provides, a method of degrading a target protein in a subject comprising administering to a subject in need thereof, a therapeutically effective amount of the compound of formula (I), (IA), (IB), (IC), (ID), (IE), (IF) or (IG) or a pharmaceutically acceptable salt or a stereoisomer or a tautomer thereof.

In one embodiment, the present invention provides a method of degrading a target protein is SMARCA2 and/or SMARCA4.

In one embodiment, the present invention provides, a method of treating or delaying progression of a disease or disorder dependent upon SMARCA2 and/or SMARCA4 in a subject comprising administering to the subject, in need thereof, a therapeutically effective amount of a compound of formula (I), (IA), (IB), (IC), (ID), (IE), (IF) or (IG) or a pharmaceutically acceptable salt or a stereoisomer or a tautomer thereof.

In one embodiment, the present invention provides a method for treating diseases or disorders dependent upon SMARCA2 and/or SMARCA4 in a subject comprising administering to the subject in need thereof a therapeutically effective amount of a compound formula (I), (IA), (IB), (IC), (ID), (IE), (IF) or (IG) or a pharmaceutically acceptable salt or a stereoisomer or a tautomer thereof.

In one embodiment, diseases or disorders that are dependent upon SMARCA2 and/or SMARCA4, include cancer.

In one embodiment, the present invention provides a method of inhibiting tumor growth in a subject afflicted with cancer comprising administering a therapeutically effective amount of a compound of formula (I), (IA), (IB), (IC), (ID), (IE), (IF) or (IG) or a pharmaceutically acceptable salt or a stereoisomer or a tautomer thereof to the subject, in need thereof.

In one embodiment, the present invention provides a pharmaceutical composition comprising a compound of formula (I), (IA), (IB), (IC), (ID), (IE), (IF) or (IG) or a pharmaceutical acceptable salt or a stereoisomer or a tautomer thereof, for use in degrading a target protein in a subject wherein the target protein is SMARCA2 and/or SMARCA4.

In one embodiment, the subject is afflicted with a disease or disorder dependent upon at least one of SMARCA2 and SMARCA4, wherein the disease or disorder is cancer.

In one embodiment, the present invention provides a use of a compound represented by formula (I), (IA), (IB), (IC), (ID), (IE), (IF) or (IG) or a pharmaceutical acceptable salt or a stereoisomer or a tautomer thereof, in the manufacture of a medicament for the treatment of disease or disorder dependent upon SMARCA2 and/or SMARCA4.

In one embodiment, disease or disorder dependent upon SMARCA2 and/or SMARCA4 is a cancer.

In any one of the preceding embodiment, cancer is selected from hematologic cancers, lung cancer, non-small cell lung cancer, acoustic neuroma, acute leukemia, acute lymphocytic leukemia, acute myelocytic leukemia, adenocarcinoma, angiosarcoma, astrocytoma, myelomonocytic, promyelocytic, acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic leukemia, granulocytic leukemia, chronic myelogenous leukemia, colon cancer, colorectal cancer, craniopharyngioma, cystadenocarcinoma, diffuse large B-cell lymphoma, dysproliferative changes, dysplasias, metaplasias, embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, head and neck cancer, hemangioblastoma, hepatoma, hepatocellular cancer, hormone insensitive prostate cancer, leiomyosarcoma, leukemia, liposarcoma, liver cancer, lymphagioendotheliosarcoma, lymphangiosarcoma, lymphoblastic leukemia, lymphoma, Hodgkin's lymphoma, non-Hodgkin's lymphoma, Burkitt's lymphoma, malignancies and hyperproliferative disorders of the bladder, breast, colon, lung, ovaries, pancreas, prostate, skin and uterus, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, malignant rhabdoid tumor (MRT), rhabdomyosarcoma, sarcoma, sebaceous gland carcinoma, seminoma, skin cancer, small cell lung carcinoma, solid tumors, carcinomas, sarcomas, small cell lung cancer, stomach cancer, squamous cell carcinoma, synovioma, sweat gland carcinoma, thyroid cancer, Waldenstrom's macroglobulinemia, testicular tumors, uterine cancer or Wilms' tumor.

In one embodiment, the cancer dependent upon SMARCA2 and/or SMARCA4 is lung cancer such as NSCLC, i.e., non-small cell lung cancer.

In one embodiment, the cancer dependent upon SMARCA2 and/or SMARCA4 is melanoma.

In one further embodiment, the cancer is a SMARCA2 and/or SMARCA4 dependent cancer.

In one embodiment, the present invention provides a compound of formula (I) or a pharmaceutical acceptable salt or a stereoisomer or a tautomer thereof, for use in the treatment of a disease or disorder dependent upon SMARCA2 and/or SMARCA4, wherein the disease or disorder dependent upon SMARCA2 and/or SMARCA4 is cancer.

In one embodiment, the present invention provides a use of compound of formula (I) or a pharmaceutical acceptable salt or a stereoisomer or a tautomer thereof, in the manufacture of a medicament for the treatment of a disease or disorder dependent upon SMARCA2 and/or SMARCA4; wherein the disease or disorder is cancer.

### Definitions

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated in order to facilitate the understanding of the present invention.

The singular forms “a”, “an” and “the” encompass plural references unless the context clearly indicates otherwise.

As used herein, the terms “optional” or “optionally” mean that the subsequently described event or circumstance may occur or may not occur and that the description includes instances where the event or circumstance occurs as well as instances in which it does not. For example, “optionally substituted alkyl” refers to an event or circumstance where the alkyl is substituted as well as the event or circumstance where the alkyl is not substituted. The term “optionally substituted alkyl” can also be referred to as ‘unsubstituted or substituted alkyl’ group, wherein the substituent(s) are as stated therein.

As used herein, the term “unsubstituted” means that there is no substituent or that the only substituents are hydrogen.

As used herein, the term “substituted” refers to moieties having substituents replacing hydrogen on one or more carbons of the backbone. It will be understood that “substitution” or “substituted with” includes the implicit proviso that such substitution is in accordance with permitted valence of the substituted atom and the substituent and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl or an acyl), a thiocarbonyl (such as a thioester, a thioacetate or a thioformate), an alkoxyl, an oxo, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heteroaryl, a heterocyclyl, an aralkyl or an aromatic or heteroaromatic moiety. It will be understood by those skilled in the art that substituents can themselves be substituted, if appropriate.

As used herein, the term “alkylenyl” refers to a divalent alkyl group as defined herein. The term “alkyl” refers to saturated aliphatic groups, including but not limited to C₁-C₁₀ straight-chain alkyl groups or C₃-C₁₀ branched-chain alkyl groups. Preferably, “alkyl” group refers to C₁-C₆ straight-chain alkyl groups or C₃-C₆ branched-chain alkyl groups. In one embodiment, the “alkyl” group refers to C₁-C₄ alkyl groups. Examples of “alkyl” include, but are not limited to, methyl, ethyl, 1-propyl, 2-propyl, isopropyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl and 4-octyl. Accordingly, examples of “alkylenyl” include, but are not limited to, —CH₂—, —CH₂CH₂—, —CH(CH₃)CH₂—, —CH₂CH₂CH₂CH₂—, —CH(CH₃)CH₂CH₂CH₂— and —CH₂CH(CH₃)CH₂CH₂—, The “alkyl” group may be optionally substituted. In one embodiment, the term “optionally substituted alkyl” can also be referred to as ‘unsubstituted or substituted alkyl’ group, wherein the substituent(s) are as stated therein.

As used herein, the term “halo” is used herein interchangeably with the term “halogen” to mean F, Cl, Br or I atoms.

As used herein, the term “amino” is art-recognized and refers to an —NH₂ group.

As used herein, the term “haloalkyl” refers to alkyl substituted with one or more halogen atoms, wherein the halo and alkyl groups are as defined above. In one embodiment, haloalkyl contains (C₁-C₆)alkyl and preferably (C₁-C₄)alkyl. Examples of “haloalkyl” include but are not limited to fluoromethyl, difluoromethyl, chloromethyl, trifluoromethyl and 2,2,2-trifluoroethyl.

As used herein, the term “hydroxyalkyl” refers to an alkyl group, as defined above, wherein one or more of the alkyl group's hydrogen atoms have been replaced with hydroxyl group. In one embodiment, hydroxyalkyl contains (C₁-C₆)alkyl and preferably (C₁-C₄)alkyl. Examples of hydroxyalkyl moieties include but are not limited to —CH₂OH, —CH₂CH₂OH, —CH₂CH₂CH₂OH, —CH₂CH(OH)CH₂OH, —CH₂CH(OH)CH₃, —CH(CH₃)CH₂OH.

As used herein, the term “hydroxy” or “hydroxyl” alone or in combination with other term(s), is art-recognized and refers to —OH.

As used herein, the term “oxo” is art-recognized and refers to ═O group.

As used herein, the term “alkoxy” refers to the group-O-alkyl, where alkyl groups are as defined above. Exemplary C₁-C₁₀ alkoxy group include but are not limited to methoxy, ethoxy, n-propoxy, n-butoxy or t-butoxy. In one embodiment, the “alkoxy” group refers to C₁-C₆ alkoxy groups. In another embodiment, the “alkoxy” group refers to C₁-C₄ alkoxy groups. An alkoxy group can be optionally substituted with one or more suitable groups. In some embodiments, alkoxy group can be unsubstituted or substituted with one or more substitutents, wherein the substitutents are described herein.

As used herein, the term “cycloalkyl” means C₃-C₁₀ saturated cyclic hydrocarbon ring. A cycloalkyl may be a single ring, which typically contains from 3 to 7 carbon ring atoms. Examples of single ring cycloalkyls include, but are not limited to, cyclopropyl, cyclobutyl, cyclopentyl, cyclohexyl and cycloheptyl. A cycloalkyl may alternatively be polycyclic or contain more than one ring. Examples of polycyclic cycloalkyls include bridged, fused and spirocyclic carbocyclyls. Accordingly, examples of ‘cycloalkylenyl include, but not limited to, cyclopropylenyl, cyclobutylenyl, cyclopentylenyl, cyclohexylenyl and cycloheptylenyl.

As used herein, the term “heterocycloalkylenyl” refers to a divalent heterocycloalkyl group as defined herein. The term “heterocycloalkyl” refers to a non-aromatic, saturated or partially saturated, bridged bicyclic, spirocyclic, monocyclic or polycyclic ring system of 3 to 15 member, unless the ring size is specifically mentioned, having at least one heteroatom or heterogroup selected from O, N and S with the remaining ring atoms being independently selected from the group consisting of carbon, oxygen, nitrogen and sulfur. The term “heterocycloalkyl” also refers to the bridged bicyclic ring system having at least one heteroatom or hetero group selected from O, N and S. Examples of “heterocycloalkyl” include, but not limited to, azetidinyl, oxetanyl, imidazolidinyl, pyrrolidinyl, oxazolidinyl, thiazolidinyl, pyrazolidinyl, tetrahydrofuranyl, piperidinyl, dihydropyridinyl, piperazinyl, tetrahydropyranyl, morpholinyl, thiomorpholinyl, 1,4-dioxanyl, dioxidothiomorpholinyl, oxapiperazinyl, oxapiperidinyl, tetrahydrofuryl, tetrahydropyranyl, tetrahydrothiophenyl, dihydropyranyl, indolinyl, indolinylmethyl, isoindolinyl, oxoisoindolinyl, dioxoisoindolinyl, aza-bicyclooctanyl, diazabicyclooctanyl, azocinyl, chromanyl, isochromanyl, xanthenyl and 2-oxa-6-azaspiro[3.3]heptanyl. Accordingly, examples of ‘heterocycloalkylenyl’ include, but not limited to, azetidinylenyl, oxetanylenyl, pyrrolidinylenyl, piperidinylenyl and piperazinylenyl. All “heterocycloalyl” and “heterocycloalkylenyl” are optionally substituted by one or more aforesaid groups.

As used herein, the term “arylenyl” refers to a divalent aryl group as defined herein. The term “aryl”, as employed herein as such or as part of another group, refers to a monocyclic, bicyclic or polycyclic aromatic hydrocarbon ring system of 6 to 14 carbon atoms. Examples of aryl groups include, but are not limited to phenyl, naphthyl, biphenyl, anthryl, biphenylenyl and acenaphthyl. Preferred aryl group is phenyl. The term “arylenyl” refers to a divalent aryl group. Accordingly, examples of arylenyl groups include, but are not limited to phenylenyl, naphthylenyl, biphenylenyl and anthrylenyl. The term “optionally substituted aryl” can also be referred to as ‘unsubstituted or substituted aryl’ group, wherein the substituent(s) are as stated therein.

As used herein, the term ‘compound(s)’ comprise(s) the compound(s) disclosed in the present invention.

As used herein, the term “salt/salts” refers to the salts derived from appropriate bases include alkali metal (e.g., sodium and potassium), alkaline earth metal (e.g., magnesium), ammonium and N⁺(C₁₋₄ alkyl)₄ salts.

As used herein, the term “comprise” or “comprising” is generally used in the sense of include, that is to say permitting the presence of one or more features or components.

As used herein, the term “or” means “and/or” unless stated otherwise.

As used herein, the term “including” as well as other forms, such as “include”, “includes” and “included” is not limiting.

As used herein, the term “composition” is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

As used herein, the term “pharmaceutical composition” refers to a composition(s) containing a therapeutically effective amount of at least one compound of formula (I) or a pharmaceutically acceptable salt thereof; and a pharmaceutically acceptable carrier.

The pharmaceutical composition(s) usually contain(s) about 1% to 99%, for example, about 5% to 75% or from about 10% to about 30% by weight of the compound of formula (I) or (II) or pharmaceutically acceptable salts thereof. The amount of the compound of formula (I) or pharmaceutically acceptable salts thereof in the pharmaceutical composition(s) can range from about 1 mg to about 1000 mg or from about 2.5 mg to about 500 mg or from about 5 mg to about 250 mg or in any range falling within the broader range of 1 mg to 1000 mg or higher or lower than the aforementioned range.

The term “tautomer” refers to compounds in which hydrogen atoms are transposed to other parts of the molecules and the chemical bonds between the atoms of the molecules are consequently rearranged. Compounds of the present invention, free form and salts thereof, may exist in multiple tautomeric forms. It is understood that all tautomeric forms, insofar as they may exist, are included within the invention. For example, pyridine or pyridyl can be optionally substituted by oxo to form a respective pyridone or pyridon-yl and may include its tautomeric form such as a respective hydroxy-pyridine or hydroxy-pyridyl, provided said tautomeric form may be obtainable.

As used herein, “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant or emulsifier that has been approved by the United States Food and Drug Administration as being acceptable for use in humans or domestic animals.

As used herein, the term “administer”, “administering,” or “administration” as used in this disclosure refers to either directly administering one or more disclosed compounds or a pharmaceutically acceptable salt of one or more disclosed compounds or a composition comprising one or more disclosed compounds to a subject or administering a prodrug derivative or analog of the compound or a pharmaceutically acceptable salt of the compound or composition to the subject, which can form an equivalent amount of active compound within the subject's body.

As used herein, the term “carrier” as used in this disclosure, encompasses carriers, excipients and diluents and means a material, composition or vehicle, such as a liquid or solid filler, diluent, excipient, solvent or encapsulating material, involved in carrying or transporting a pharmaceutical agent from one organ or portion of the body to another organ or portion of the body of a subject.

As used herein, the term “treat”, “treating” and “treatment” refer to a method of alleviating or abrogating a disease and/or its attendant symptoms.

As used herein, the term “prevent”, “preventing” and “prevention” refer to a method of preventing the onset of a disease and/or its attendant symptoms or barring a subject from acquiring a disease. As used herein, “prevent”, “preventing” and “prevention” also include delaying the onset of a disease and/or its attendant symptoms and reducing a subject's risk of acquiring a disease.

As used herein, the term “subject” that may be interchangeable with ‘patient’, refers to an animal, preferably a mammal and most preferably a human.

As used herein, the term, “therapeutically effective amount” refers to an amount of a compound of formula (I) or a pharmaceutically acceptable salt or a stereoisomer or a tautomer thereof; or a composition comprising the compound of formula (I) or a pharmaceutically acceptable salt or a stereoisomer or a tautomer thereof, effective in producing the desired therapeutic response in a particular patient suffering from a diseases or disorder, in particular their use in diseases or disorder associated with cancer. Particularly, the term “therapeutically effective amount” includes the amount of the compound of formula (I) or a pharmaceutically acceptable salt or a stereoisomer or a tautomer thereof, when administered, that induces a positive modification in the disease or disorder to be treated or is sufficient to prevent development of or alleviate to some extent, one or more of the symptoms of the disease or disorder being treated in a subject. In respect of the therapeutic amount of the compound, the amount of the compound used for the treatment of a subject is low enough to avoid undue or severe side effects, within the scope of sound medical judgment can also be considered. The therapeutically effective amount of the compound or composition will be varied with the particular condition being treated, the severity of the condition being treated or prevented, the duration of the treatment, the nature of concurrent therapy, the age and physical condition of the end user, the specific compound or composition employed the particular pharmaceutically acceptable carrier utilized.

As used herein, the term “pharmaceutically acceptable salt” refers to a product obtained by reaction of the compound of the present invention with a suitable acid or a base. Pharmaceutically acceptable salts of the compounds of this invention include those derived from suitable inorganic bases such as Li, Na, K, Ca, Mg, Fe, Cu, Al, Zn and Mn salts; Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain compounds of the invention (compound of formula (I)) can form pharmaceutically acceptable salts with various organic bases such as lysine, arginine, guanidine, diethanolamine or metformin. Suitable base salts include, but are not limited to, aluminum, calcium, lithium, magnesium, potassium, sodium or zinc salts.

“Pharmaceutically acceptable” means that, which is useful in preparing a pharmaceutical composition that is generally safe, non-toxic and neither biologically nor otherwise undesirable and includes that which is acceptable for veterinary as well as human pharmaceutical use.

The present invention also provides methods for formulating the disclosed compounds as for pharmaceutical administration.

In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

The term “cancer” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites and are likely to recur after attempted removal and to cause the death of the patient unless adequately treated. As used herein, the term neoplasia is used to describe all cancerous disease states and embraces or encompasses the pathological process associated with malignant hematogenous, ascitic and solid tumors. Exemplary cancers which may be treated by the present compounds include squamous-cell carcinoma, basal cell carcinoma, adenocarcinoma, hepatocellular carcinomas, hematologic cancers and renal cell carcinomas, cancer of the bladder, bowel, breast, cervix, colon, esophagus, head, kidney, liver, lung, neck, ovary, pancreas, prostate and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using compounds according to the present invention include, for example, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL and Philadelphia chromosome positive CML.

The term “stereoisomers” refers to any enantiomers, diastereoisomers or geometrical isomers of the compound of formula (I), wherever they are chiral or when they bear one or more double bonds. When the compounds of the formula (I) and related formulae are chiral, they can exist in racemic or in optically active form. It should be understood that the invention encompasses all stereochemical isomeric forms, including diastereomeric, enantiomeric and epimeric forms, as well as d-Isomers and I-Isomers and mixtures thereof. Individual stereoisomers of compounds can be prepared synthetically from commercially available starting materials which contain chiral centres or by preparation of mixtures of enantiomeric products followed by separation such as conversion to a mixture of diastereomers followed by separation or recrystallization, chromatographic techniques, direct separation of enantiomers on chiral chromatographic columns or any other appropriate method known in the art. Starting compounds of particular stereochemistry are either commercially available or can be made and resolved by techniques known in the art. Additionally, the compounds of the present invention may exist as geometric Isomers. The present invention includes all cis, trans, syn, anti, entgegen I and zusammen (Z) Isomers as well as the appropriate mixtures thereof.

The term “enantiomers” refers to a pair of stereoisomers which are non-superimposable mirror images of one another. The term “enantiomer” refers to a single member of this pair of stereoisomers. The term “racemic” refers to a 1:1 mixture of a pair of enantiomers. The disclosure includes enantiomers of the compounds described herein. Each compound herein disclosed includes all the enantiomers that conform to the general structure of the compound. The compounds may be in a racemic or enantiomerically pure form or any other form in terms of stereochemistry. In some embodiments the compounds are the (S)-enantiomer.

The term “diastereomers” refers to the set of stereoisomers which cannot be made superimposable by rotation around single bonds. For example, cis- and trans-double bonds, endo- and exo-substitution on bicyclic ring systems and compounds containing multiple stereogenic centres with different relative configurations are considered to be diastereomers. The term “diastereomer” refers to any member of this set of compounds. In some examples presented, the synthetic route may produce a single diastereomer or a mixture of diastereomers. The disclosure includes diastereomers of the compounds described herein.

The compounds of the present invention may be used as single drug or as a pharmaceutical composition in which the compound is mixed with various pharmacologically acceptable materials.

The compounds of the invention are typically administered in the form of a pharmaceutical composition. Such compositions can be prepared using procedures well known in the pharmaceutical art and comprise at least one compound of the invention. The pharmaceutical composition of the present patent application comprises one or more compounds described herein and one or more pharmaceutically acceptable excipients. Typically, the pharmaceutically acceptable excipients are approved by regulatory authorities or are generally regarded as safe for human or animal use. The pharmaceutically acceptable excipients include, but are not limited to, carriers, diluents, glidants and lubricants, preservatives, buffering agents, chelating agents, polymers, gelling agents, viscosifying agents and solvents.

The pharmaceutical composition can be administered by oral, parenteral or inhalation routes. Examples of the parenteral administration include administration by injection, percutaneous, transmucosal, transnasal and transpulmonary administrations.

Examples of suitable carriers include, but are not limited to, water, salt solutions, alcohols, polyethylene glycols, peanut oil, olive oil, gelatin, lactose, terra alba, sucrose, dextrin, magnesium carbonate, sugar, amylose, magnesium stearate, talc, gelatin, agar, pectin, acacia, stearic acid, lower alkyl ethers of cellulose, silicic acid, fatty acids, fatty acid amines, fatty acid monoglycerides and diglycerides, fatty acid esters and polyoxyethylene.

The pharmaceutical composition may also include one or more pharmaceutically acceptable auxiliary agents, wetting agents, suspending agents, preserving agents, buffers, sweetening agents, flavouring agents, colorants or any combination of the foregoing.

The pharmaceutical compositions may be in conventional forms, for example, tablets, capsules, solutions, suspensions, injectables or products for topical application. Further, the pharmaceutical composition of the present invention may be formulated so as to provide desired release profile.

Administration of the compounds of the invention, in pure form or in an appropriate pharmaceutical composition, can be carried out using any of the accepted routes of administration of pharmaceutical compositions. The route of administration may be any route which effectively transports the active compound of the patent application to the appropriate or desired site of action. Suitable routes of administration include, but are not limited to oral, nasal, buccal, dermal, intradermal, transdermal, parenteral, rectal, subcutaneous, intravenous, intraurethral, intramuscular or topical.

Solid oral formulations include, but are not limited to, tablets, capsules (soft or hard gelatin), dragees (containing the active ingredient in powder or pellet form), troches and lozenges.

Liquid formulations include, but are not limited to, syrups, emulsions and sterile injectable liquids, such as suspensions or solutions.

Topical dosage forms of the compounds include ointments, pastes, creams, lotions, powders, solutions, eye or ear drops, impregnated dressings and may contain appropriate conventional additives such as preservatives, solvents to assist drug penetration.

The pharmaceutical compositions of the present patent application may be prepared by conventional techniques known in literature.

Suitable doses of the compounds for use in treating the diseases or disorders described herein can be determined by those skilled in the relevant art. Therapeutic doses are generally identified through a dose ranging study in humans based on preliminary evidence derived from the animal studies. Doses must be sufficient to result in a desired therapeutic benefit without causing unwanted side effects. Mode of administration, dosage forms and suitable pharmaceutical excipients can also be well used and adjusted by those skilled in the art. All changes and modifications are envisioned within the scope of the present patent application.

According to one embodiment, the compounds of the present invention can also contain unnatural proportions of atomic isotopes at one or more of the atoms that constitute such compounds. For example, the present invention also embraces isotopically-labeled variants of the present invention which are identical to those recited herein, but for the fact that one or more atoms of the compound are replaced by an atom having the atomic mass or mass number different from the predominant atomic mass or mass number usually found in nature for the atom. All isotopes of any particular atom or element as specified are contemplated within the scope of the compounds of the invention and their uses. Exemplary isotopes that can be incorporated into compounds of the invention include isotopes of hydrogen, carbon, nitrogen, oxygen, phosphorous, sulfur, fluorine, chlorine and iodine, such as ²H (“D”), ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically labeled compounds of the present inventions can generally be prepared by following procedures analogous to those disclosed in the schemes and/or in the examples herein below, by substituting an isotopically labeled reagent for a non-isotopically labeled reagent.

### EXPERIMENTAL

The abbreviations used in the entire specification is summarized below with their meaning.

MeOH—Methanol, EtOH—Ethanol, DCM—Dichloromethane, DMF—N,N-Dimethylformamide, EtOAc—Ethyl acetate, THF—Tetrahydrofuran, DME—1,2-Dimethoxyethane, DMSO—Dimethyl sulfoxide DIPEA—N,N-Diisopropylethylamine, NCS—N-chloro succinimide, HATU—(1-[Bis(dimethylamino)methylene]-1H-1,2,3-triazolo[4,5-b]pyridinium 3-oxid hexafluoro phosphate), KOAc—Potassium acetate, Na₂S—₄—Sodium sulphate, Na₂CO₃—Sodium carbonate, K₂CO₃—Potassium carbonate, Cs₂CO₃—Cesium carbonate, KO^{t}Bu—Potassium tert-butoxide, Pd(dppf)Cl₂·DCM—[1,1′-Bis(diphenylphosphino)ferrocene]-dichloropalladium (II) dichloromethane complex, NaoMe—Sodium methoxide, BINAP—(2,2′-bis(diphenylphosphino)-1,1′-binaphthyl), H₂SO₄—Sulfuric acid, NaHCO₃—Sodium bicarbonate, TEA—Triethyl amine; DEA-Diethyl amine, LiOH H₂O Lithium hydroxide EDC·HCl—1-(3-Dimethylaminopropyl)-3-ethylcarbodiimide. hydrochloride, DAST—diethylaminosulfur trifluoride, PPTs—Pyridinium p-toluenesulfonate, NaH—Sodium hydride, NH₄OH-Ammonium hydroxide, NaOH-Sodium hydroxide, HCl-Hydrochloric acid, Pd(pph₃)₂Cl₂, DCM—Bis(triphenylphosphine)-palladium (II) dichloride Dichloromethane complex; Pd(OAc)₂—Palladium (II) acetate, mL—Milliliter, TLC—Thin layer chromatography, RT—Room temperature, h—Hour, N—Normality, M—Molarity, ¹HNMR—Proton nuclear magnetic resonance, DMSO-d₆—Deuterated Dimethyl sulfoxide, CDCl₃—Deuterated chloroform, CD₃OD—Deuterated Methanol, s—Singlet, d—Doublet, t—Triplet, m—Multiplet, H—Proton, MHz—Mega hertz, Hz—Hertz, Ppm—Parts per million, Bs—Broad singlet, HPLC—High-performance liquid chromatography, LCMS—Liquid chromatography Mass spectroscopy, g—Gram, mmol—Milli mol and ° C.—degree centigrade.

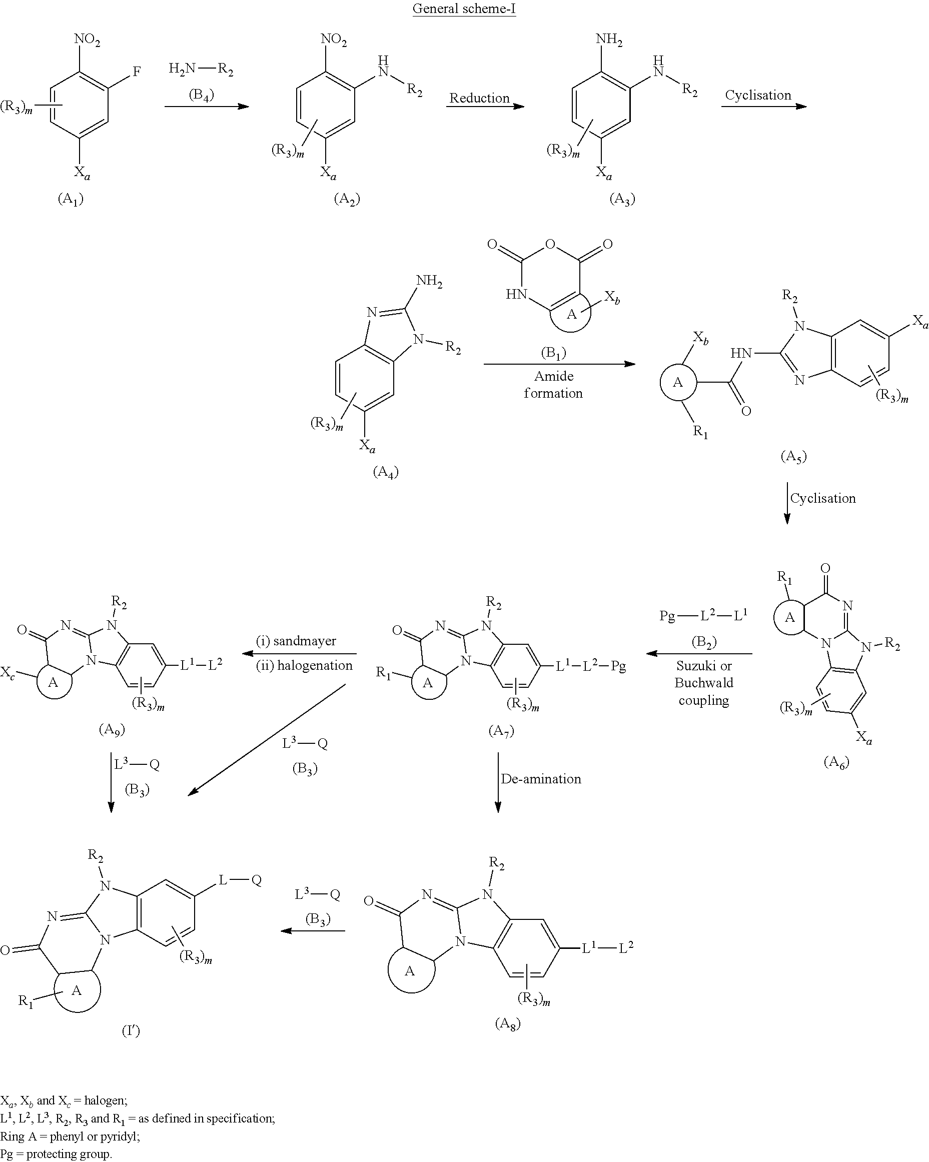

The general scheme for the synthesis of compound represented by formula (I′) is depicted in above scheme. The compound of formula (A1) was reacted with the compound of formula (B4) in suitable solvent to yield compound of formula (A2) which upon further reduction in presence of suitable reducing agent and solvent gives compound of formula (A3). The compound of formula (A3) undergoes cyclization reaction to result compound of formula (A4). The compound of formula (A4) was reacted with the compound of formula (B1) with suitable solvent in high temperature to result in amide compound of formula (As) which further undergoes cyclization reaction in the presence of suitable reagent and solvent to provide compound of formula (A6). The compound of formula (A6) undergoes Suzuki or Buchwald coupling with the compound of formula (B2) to provide compound of formula (A7) which further undergoes Sandmeyer reaction or halogenation reaction in presence of sodium nitrite and copper chloride to result the compound of formula (A9). The compound of formula (A7) undergoes de-amination rection in presence of sodium nitrite to result the compound of formula (As). The compound of formula (A7), (As) and (A9) was reacted with the compound of formula (B3) in appropriate solvent resulting in the formation of the compound of formula (I′).

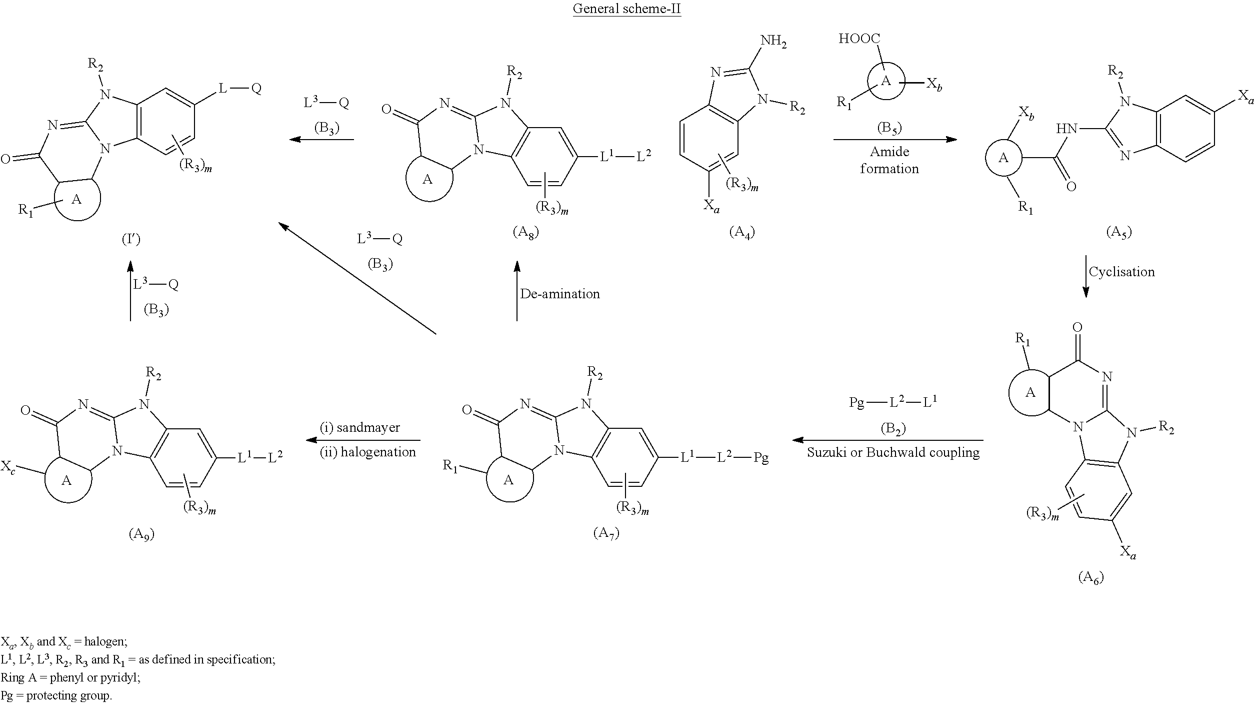

The general scheme for the synthesis of compound represented by formula (I′) is depicted in above scheme. The compound of formula (As) was obtained by the reaction of compound of formula (A4) with the compound of formula (B5) with suitable solvent in high temperature. The compound formula (As) undergoes cyclization reaction in the presence of suitable reagent and solvent to provide compound of formula (A6) which further undergoes Suzuki or Buchwald coupling with the compound of formula (B2) to provide compound of formula (A7). The compound of formula (A7) undergoes Sandmeyer reaction or halogenation reaction in presence of sodium nitrite and copper chloride to result the compound of formula (A9). The compound of formula (A7) undergoes de-amination rection in presence of sodium nitrite to result the compound of formula (As). The compound of formula (A7), (A8) and (A9) was reacted with the compound of formula (B3) in appropriate solvent result in the formation of the compound of formula (I′).

### Intermediate 1: Synthesis of 3-(4-(piperazin-1-yl)phenyl)piperidine-2,6-dione hydrochloride

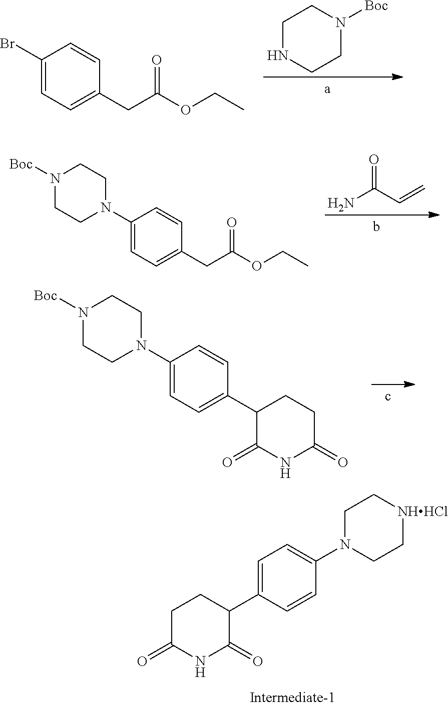

### Step-a: Synthesis of tert-butyl 4-(4-(2-ethoxy-2-oxoethyl)phenyl) piperazine-1-carboxylate

To a stirred solution of ethyl 2-(4-bromophenyl)acetate (2 g, 8.22 mmol), tert-butyl piperazine-1-carboxylate (1.53 g, 8.22 mmol) in toluene (15 mL) was added Cs₂CO₃ (5.36 g, 16.45 mmol) and degassed with nitrogen for 10 min followed by addition of Ru-Phos Pd-G₂ (0.32 g, 0.41 mmol) and the reaction mixture was heated for 12 h at 110° C. in sealed tube. Once the reaction was completed (monitored by TLC), the reaction mixture was dissolved in 10% MeOH/DCM and passed through celite bed. Filtrate was collected and concentrated under reduced pressure to obtain crude compound which was purified by combi flash column chromatography using 20-25% ethyl acetate in hexane as eluent to afford the title compound as off-white solid (2.1 g, 73.2%). ¹H NMR (400 MHz, DMSO-d₆): δ 7.10 (d, J=8.4 Hz, 2H), 6.89 (d, J=8.4 Hz, 2H), 4.04 (q, J=7.2 Hz, 2H), 3.52 (s, 2H), 3.43 (m, 4H), 3.05 (m, 4H), 1.41 (s, 9H), 1.16 (t, J=7.2 Hz, 3H); LC-MS: m/z 349.2 (M+H).

### Step-b: Synthesis of tert-butyl 4-(4-(2,6-dioxopiperidin-3-yl)phenyl) piperazine-1-carboxylate

To a stirred solution of tert-butyl 4-(4-(2-ethoxy-2-oxoethyl)phenyl) piperazine-1-carboxylate (2 g, 5.74 mmol) and acrylamide (0.36 g, 5.16 mmol) were taken in THF (20 mL) was added KO Bu (0.70 g, 6.31 mmol) and the reaction mixture was heated for 12 h at 50° C. Once the reaction was completed (monitored by TLC), the reaction mixture was quenched with ice cold water and extracted with EtOAc. The combined organic layer was washed with water, brine, dried over anhydrous sodium sulphate and concentrated under vacuum to give the residue which was purified by combi flash column chromatography using 40-60% ethyl acetate in hexane as eluent to afford the title compound as off-white solid (0.82 g, 38%). ¹H NMR (400 MHz, DMSO-d₆): δ 10.77 (s, 1H), 7.06 (d, J=8.8 Hz, 2H), 6.90 (d, J=8.8 Hz, 2H), 3.73 (m, 1H), 3.44 (m, 4H), 3.06 (m, 4H), 2.69-2.54 (m, 1H), 2.48-2.42 (m, 1H), 2.18-2.07 (m, 1H), 2.03-1.95 (m, 1H), 1.41 (s, 9H); LC-MS: m/z 374.3 (M+H).

### Step-c: Synthesis of 3-(4-(piperazin-1-yl)phenyl)piperidine-2,6-dione hydrochloride

To a stirred solution of tert-butyl 4-(4-(2,6-dioxopiperidin-3-yl)phenyl) piperazine-1-carboxylate (0.82 g, 2.19 mmol) in DCM (10 mL) was added 4 (N) dioxane HCl (10 mL) at 0° C. and then slowly brought into RT and stirred at RT for 16 h. Reaction mixture was evaporated under reduced pressure, the resultant residue was washed with diethyl ether and dried under vacuum to afford the title compounds yellowish solid (0.8 g, crude). 1H NMR (400 MHz, DMSO-d₆): δ 10.78 (s, 1H), 9.35 (bs, 2H), 7.10 (d, J=8.4 Hz, 2H), 6.96 (d, J=8.4 Hz, 2H), 3.78-3.74 (m, 1H), 3.37 (m, 4H), 3.19 (m, 4H), 2.69-2.56 (m, 1H), 2.47-2.41 (m, 1H), 2.21-2.09 (m, 1H), 2.05-1.95 (m, 1H); LC-MS: m/z 274.2 (M+H).

### Intermediate-2: Synthesis of 3-(4-(Piperidin-4-yl)phenoxy)piperidine-2,6-dione hydrochloride

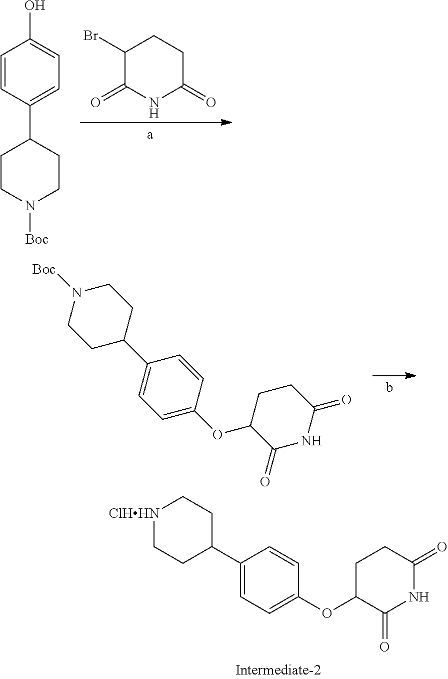

### Step-a: Synthesis of tert-butyl 4-(4-((2,6-dioxopiperidin-3-yl)oxy)phenyl)piperidine-1-carboxylate

To a stirred solution of tert-butyl 4-(4-hydroxyphenyl)piperidine-1-carboxylate (synthesized using Ref. chem comm 2018, 54, 46,-49) (2.0 g, 7.21 mmol) in DMF (20 mL) was added NaH (0.43 g, 18.02 mmol) at 0° C. temperature and stirred for 30 mins. 3-bromopiperidine-2,6-dione (1.66 g, 8.65 mmol) was added to the reaction mixture and stirred at RT for 2 h. After completion of the reaction the reaction mixture was poured in ice cold water and extracted with 2×100 mL of ethyl acetate, evaporated. The crude obtained was purified first by combi flash column chromatography using 20-30% ethyl acetate in hexane to afford the title compound colourless solid (1.1 g, 39%). ¹H NMR (400 MHz, DMSO-d₆): □ 10.91 (s, 1H), 7.14 (d, J=8.8 Hz, 2H), 6.93 (d, J=8.4 Hz, 2H), 5.15-5.13 (m, 1H), 4.10-3.90 (m, 2H), 2.84-2.72 (m, 2H), 2.68-2.55 (m, 2H), 2.20-2.11 (m, 3H), 1.73-1.69 (m, 2H), 1.51-1.42 (m, 2H), 1.41 (s, 9H). LCMS: m/z 387.1 (M−H).

### Step-b: Synthesis of 3-(4-(piperidin-4-yl)phenoxy)piperidine-2,6-dione hydrochloride

To a stirred solution of tert-butyl 4-(4-((2,6-dioxopiperidin-3-yl)oxy)phenyl)piperidine-1-carboxylate (1.1 g, 2.83 mmol) in DCM (10 mL) was added 4N dioxane hydrochloride (10 mL) at 0° C. and then slowly brought into RT and stirred for 4 h. Reaction mixture was evaporated under reduced pressure, the resultant residue was washed with diethyl ether and dried under vacuum to afford the title compound as yellowish solid (0.81 g, crude). ¹H NMR (400 MHz, DMSO-d₆): 10.93 (s, 1H), 8.91 (bs, 1H), 8.71 (bs, 1H), 7.13 (d, J=8.8 Hz, 2H), 6.97 (d, J=8.4 Hz, 2H), 5.18-5.15 (m, 1H), 3.34-3.31 (m, 2H), 2.98-2.93 (m, 2H), 2.78-2.68 (m, 2H), 2.63-2.61 (m, 1H), 2.17-2.12 (m, 2H), 1.91-1.87 (m, 2H), 1.85-1.78 (m, 2H). LCMS: m/z 289.1 (M+H).

### Intermediate 3: 3-((3-Fluoro-4-(piperidin-4-yl)phenyl)amino)piperidine-2,6-dione hydrochloride

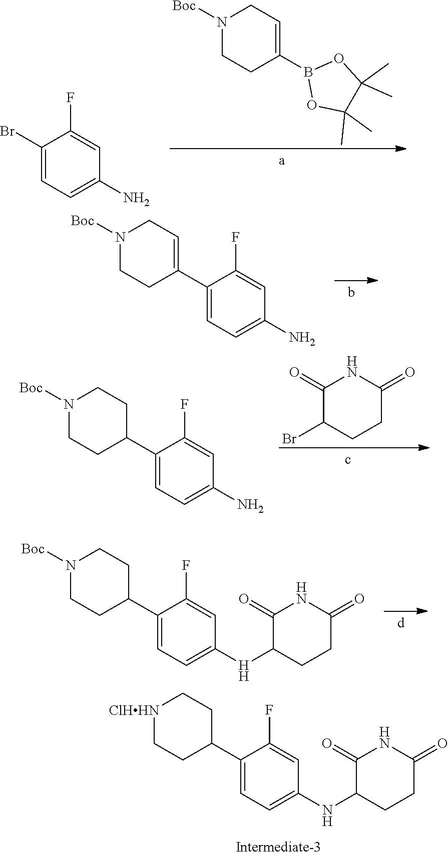

### Step-a: Synthesis of tert-butyl 4-(4-amino-2-fluorophenyl)-3,6-dihydropyridine-1(2H)-carboxylate

To a stirred solution of 4-bromo-3-fluoroaniline (25.0 g, 131.56 mmol), tert-butyl 4-(4,4,5,5-tetramethyl-1,3,2-dioxaborolan-2-yl)-3,6-dihydropyridine-1(2H)-carboxylate (44.75 g, 144.72 mmol) and potassium carbonate (54.5 g, 394.69 mmol) in 1,4-dioxane (200 mL) and water (50 ml) was purged with nitrogen for 10 minutes followed by Pd(dppf)Cl₂·DCM (8.05 g, 816.60 mmol) was added. The reaction mixture was heated to 110° C. for 16 h. Once the reaction was completed (monitored by TLC), the reaction mixture was diluted with EtOAc and washed with water, brine, dried over anhydrous sodium sulphate and concentrated under vacuum to give the residue which was purified by combi flash column chromatography using 20-30% ethyl acetate in hexane as eluent to afford the title compound as yellow solid (38 g, 98.8%). ¹H NMR (400 MHz, DMSO-d6): δ 6.98 (t, J=8.4 Hz, 1H), 6.35-6.27 (m, 2H), 5.78 (bs, 1H), 5.41 (s, 2H), 3.92 (bs, 2H), 3.48 (t, J=5.6 Hz, 2H), 2.34 (bs, 2H), 1.42 (s, 9H); LC-MS: m/z 293.1 (M+H).

### Step-b: Synthesis of tert-butyl 4-(4-amino-2-fluorophenyl)piperidine-1-carboxylate

To a stirred solution of tert-butyl 4-(4-amino-2-fluorophenyl)-3,6-dihydropyridine-1(2H)-carboxylate (10.0 g, 34.20 mmol) in ethanol (100 mL) and THF (10 mL) were added 10% Pd/C (3.64 g, 34.2 mmol) and the reaction mixture was stirred for 16 h in presence of hydrogen bladder. Once the reaction was completed (monitored by TLC), the reaction mixture was filtered through celite bed and washed with EtOAc, concentrated under vacuum to afford the title compound as yellow solid (10 g, crude). ¹H NMR (400 MHz, CDCl₃): δ 6.89 (t, J=8.4 Hz, 1H), 6.33-6.25 (m, 2H), 5.18 (bs, 1H), 4.04 (d, J=10.8 Hz, 2H), 3.93 (s, 1H), 2.78-2.72 (m, 2H), 1.63 (d, J=12.0 Hz, 2H), 1.50-1.42 (m, 2H), 1.41 (s, 9H); LC-MS: m/z 195.2 (M+H−100).

### Step-c: Synthesis of tert-butyl 4-(4-((2,6-dioxopiperidin-3-yl)amino)-2-fluorophenyl)piperidine-1-carboxylate

To a stirred solution of tert-butyl 4-(4-amino-2-fluorophenyl)piperidine-1-carboxylate (2.0 g, 6.79 mmol) and 3-bromopiperidine-2,6-dione (1.95 g, 10.19 mmol) in 1,4-dioxane (20 mL) was added DIPEA (3.51 g, 27.17 mmol), The reaction mixture was heated to 110° C. for 16 h. Once the reaction was completed (monitored by TLC), the reaction mixture was diluted with EtOAc, washed with water, brine, dried over anhydrous sodium sulphate and concentrated under vacuum to give the residue which was purified by combi flash column chromatography using 40-50% ethyl acetate in hexane as eluent to afford the title compound as yellow solid (0.350 g, 13%). ¹H NMR (400 MHz, CDCl₃): δ 10.78 (s, 1H), 6.98 (t, J=8.4 Hz, 1H), 6.46-6.42 (m, 2H), 6.02 (d, J=8.0 Hz, 2H), 4.32-4.29 (m, 1H), 4.05 (d, J=11.2 Hz, 2H), 2.82-2.67 (m, 3H), 2.62-2.52 (m, 2H), 2.12-2.05 (m, 1H), 1.91-1.82 (m, 1H), 1.65 (d, J=10 Hz, 2H), 1.53-1.43 (m, 2H), 1.41 (s, 9H); LC-MS: m/z 306.2 (M+H−100).

### Step-d: Synthesis of 3-((3-fluoro-4-(piperidin-4-yl)phenyl)amino)piperidine-2,6-dione hydrochloride

To a stirred solution of tert-butyl 4-(4-((2,6-dioxopiperidin-3-yl)amino)-2-fluorophenyl)piperidine-1-carboxylate (1.2 g, 2.96 mmol) in DCM (12 mL) was added 4N dioxane HCl (12 mL) at 0° C. and stirred for 16 h at RT under nitrogen atmosphere. Then the reaction mixture was concentrated under vacuum to get crude product. The obtained crude product was triturated with n-pentane and then dried under vacuum to afford pure title compound as pale yellow solid (1 g). ¹H NMR (400 MHz, DMSO-d₆): 10.79 (s, 1H), 8.89 (bs, 1H), 8.68 (bs, 1H), 6.94 (t, J=8.8 Hz, 1H), 6.50-6.45 (m, 2H), 4.34-4.30 (m, 1H), 3.24 (d, J=15.2 Hz, 2H), 3.05-2.89 (m, 3H), 2.79-2.67 (m, 1H), 2.63-2.55 (m, 1H), 2.12-2.04 (m, 1H), 1.93-1.77 (m, 5H); LC-MS: m/z 306.1 (M+H).

### Intermediate-4: Synthesis of 5-Bromo-2H-benzo[d][1,3]oxazine-2,4(1H)-dione

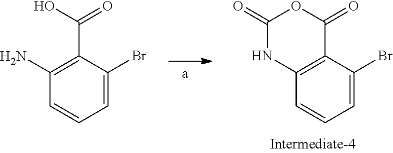 [SMILES: Nc1cccc(Br)c1C(=O)O ; O=c1[nH]c2cccc(Br)c2c(=O)o1]

### Step a: Synthesis of 5-bromo-2H-benzo[d][1,3]oxazine-2,4(1H)-dione

To a stirred solution of 2-amino-6-bromobenzoic acid (10 g, 46.28 mmol) in THF (100 mL) was added triphosgene (13.73 g, 46.28 mmol) at 0° C. and stirred at RT for 1 h. Once the reaction was completed (monitored by TLC), the reaction mixture was quenched with cold water and stirred for 1 h, the solid so formed was filtered and dried under vacuum to afford the title compounds as off white solid (11 g, crude). ¹H NMR (400 MHz, DMSO-d₆): 11.83 (s, 1H), 7.58-7.51 (m, 2H), 7.15 (dd, J=8, 1.6 Hz, 1H); LC-MS: m/z 242.0 (M+H).

### Intermediate-5: Synthesis of 4-Amino-7-cyclopentyl-9-(1,4-dioxa-8-azaspiro[4.5]decan-8-yl)benzo[4,5]imidazo[1,2-a]quinazolin-5(7H)-one

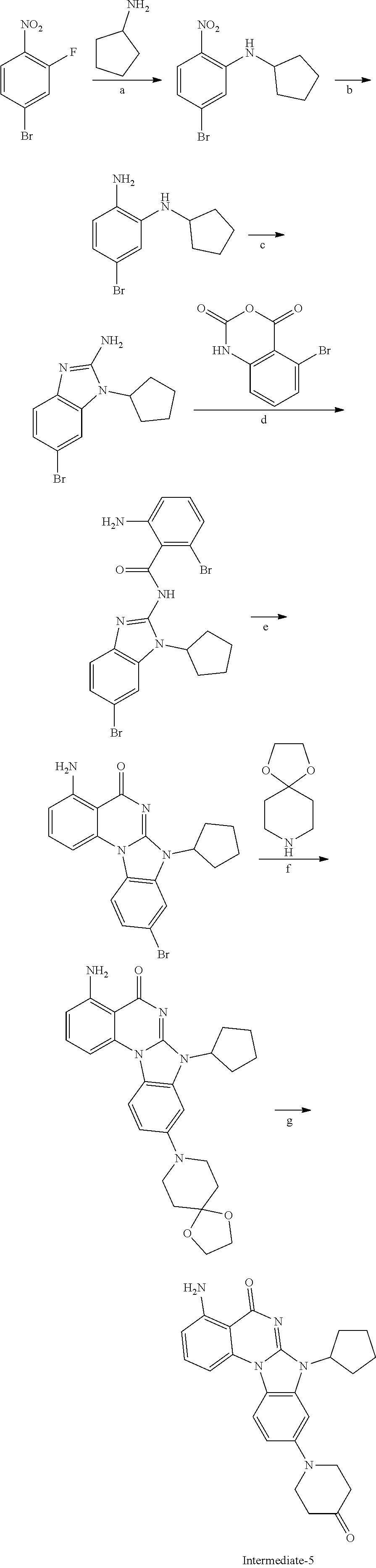

### Step a: Synthesis of 5-bromo-N-cyclopentyl-2-nitroaniline

To a stirred solution of 4-bromo-1-fluoro-2-nitrobenzene (16 g, 72.72 mmol) in DMF (150 mL) were added cyclopentanamine (6.19 g, 72.72 mmol) and K₂CO₃ (20.1 g, 145.45 mmol) at RT and heated at 90° C. for 4 h. Once the reaction was completed (monitored by TLC), the reaction mixture was quenched with cold water and stirred for 1 h, the solid so formed was filtered and dried under vacuum to afford the title compounds as yellow solid (22 g, crude). ¹H NMR (400 MHz, DMSO-d₆): 7.99 (d, J=8.8 Hz, 2H), 7.28 (d, J=2.0 Hz, 1H), 6.85 (dd, J=8.8, 2.0 Hz, 1H), 4.13-4.06 (m, 1H), 2.09-2.02 (m, 2H), 1.73-1.51 (m, 6H); LC-MS: m/z 285.1 (M+H).

### Step b: Synthesis of 5-bromo-N1-cyclopentylbenzene-1,2-diamine

To a stirred solution of 5-bromo-N-cyclopentyl-2-nitroaniline (22 g, 77.15 mmol) in methanol (20 mL) and THF (30 mL) mixture was added NH₄OH (41.27 g, 771.55 mmol) in water (20 mL) followed by addition of Zinc (50.44 g, 771.55 mmol) portion wise and stirred at RT for 10 mins. After completion of the reaction (monitored by TLC), reaction mixture was passed through celite and thoroughly washed with EtOAc. Filtrate was washed with Sat. bicarbonate solution, water and brine solution and dried over Na₂SO₄. Organic layer was concentrated to afford the title compound as brick red sticky solid (19 g, crude). ¹H NMR (400 MHz, DMSO-d₆): 6.51-6.44 (m, 3H), 4.70 (bs, 2H), 4.54 (d, J=6.0 Hz, 1H), 3.69-3.64 (m, 1H), 1.99-1.87 (m, 2H), 1.75-1.41 (m, 6H); LC-MS: m/z 255.0 (M+H).

### Step c: Synthesis of 6-bromo-1-cyclopentyl-1H-benzo[d]imidazol-2-amine

To a stirred solution of crude 5-bromo-N1-cyclopentylbenzene-1,2-diamine (19 g, 74.46 mol) in methanol (200 mL) at 0° C. was added cyanogen bromide (6.0 g, 56.7 mol) portion wise and stirred at RT for 4 h. Once the reaction was completed (monitored by TLC), the reaction mixture was quenched with saturated bicarbonate solution and stirred for 30 mins, solid so formed was filtered and dried under vacuum to afford the title compounds as brick red solid (20 g, 98% yield over three steps). ¹H NMR (400 MHz, DMSO-d₆): 7.26 (t, J=1.2 Hz, 1H), 7.06 (d, J=1.2 Hz, 2H), 6.48 (s, 2H), 4.69 (quin, J=8.8 Hz, 1H), 2.03-1.86 (m, 6H), 1.71-1.62 (m, 2H); LC-MS: m/z 280.1 (M+H).

### Step d: Synthesis of 2-amino-6-bromo-N-(6-bromo-1-cyclopentyl-1H-benzo[d]imidazol-2-yl)benzamide

To a stirred solution of 6-bromo-1-cyclopentyl-1H-benzo[d]imidazol-2-amine (8 g, 28.55 mmol) in dioxane (80 mL) was added 5-bromo-2H-benzo[d][1,3]oxazine-2,4(1H)-dione (8.63 g, 35.69 mmol) and the reaction mixture was heated at 100° C. for 6 h. Once the reaction was completed (monitored by TLC), the reaction mixture was concentrated under vacuum to get crude material which was purified by combi flash column chromatography using 2-4% methanol in DCM as eluent to afford the title compounds as brick red solid (5 g, 58.5%). ¹H NMR (400 MHz, DMSO-d₆): 12.92 (br s, 1H), 7.70 (d, J=1.2 Hz, 1H), 7.52 (d, J=4.4 Hz, 1H), 7.42 (dd, J=8.4, 1.6 Hz, 1H), 6.94-6.90 (m, 1H), 6.76 (dd, J=8.0, 0.8 Hz, 1H), 6.68 (dd, J=8.0, 0.8 Hz, 1H), 5.49 (s, 2H), 5.23-5.15 (m, 1H), 2.22-2.05 (m, 2H), 2.03-1.87 (m, 4H), 1.72-1.60 (m, 2H). LC-MS: m/z 477.1 (M+H).

### Step e: Synthesis of 4-amino-9-bromo-7-cyclopentylbenzo[4,5]imidazo[1,2-a]quinazolin-5(7H)-one

To a stirred solution of 2-amino-6-bromo-N-(6-bromo-1-cyclopentyl-1H-benzo[d]imidazol-2-yl)benzamide (5 g, 10.45 mmol) in DMSO (50 mL) was added N,N,N′,N′-Tetramethylethane-1,2-diamine (0.24 g, 2.09 mmol), copper (I) iodide (0.99 g, 5.22 mmol) and Cs₂CO₃ (5.11 g, 15.63 mmol) at RT and the reaction mixture was degassed with argon for 5 minutes then heated at 120° C. for 30 minutes. Once the reaction was completed (monitored by TLC), the reaction mixture was diluted with 10% methanol in DCM and filtered through celite and thoroughly washed with 10% methanol in DCM. The combined organic layer was concentrated under vacuum to get crude material which was purified by combi flash column chromatography using 3-5% methanol in DCM as eluent to afford the title compounds as yellowish green solid (2.1 g, 50.5%). ¹H NMR (400 MHz, DMSO-d₆): δ 8.24 (d, J=8.4 Hz, 1H), 7.86 (s, 1H), 7.50 (d, J=8.4 Hz, 1H), 7.46-7.38 (m, 1H), 7.32 (d, J=8.0 Hz, 1H), 6.69 (d, J=8.4 Hz, 1H), 5.19 (quin, J=8.4 Hz, 1H), 2.23-2.22 (m, 2H), 2.00 (br s, 4H), 1.71 (br d, J=4 Hz, 2H). LC-MS: m/z 397.1 (M+H).

### Step f: Synthesis of 4-amino-7-cyclopentyl-9-(1,4-dioxa-8-azaspiro[4.5]decan-8-yl)benzo[4,5]imidazo[1,2-a]quinazolin-5(7H)-one

To a stirred solution of 4-amino-9-bromo-7-cyclopentylbenzo[4,5]imidazo[1,2-a]quinazolin-5(7H)-one (4.5 g, 11.32 mmol) and 1,4-dioxa-8-azaspiro[4.5]decane (4.05 g, 28.31 mmol) in THF (90 mL) was degassed with nitrogen for 15 min, followed by LiHMDS (79.2 mL, 79.2 mmol) and RuPhos Pd G4 (0.96 g, 1.13 mmol) were added and the reaction mixture was heated at 80° C. for 20 mins in sealed tube. Once the reaction was completed (monitored by TLC), the reaction mixture was diluted with DCM and quenched with NH₄Cl solution and extracted with DCM. The combined organic layer was washed with water, brine, dried over anhydrous sodium sulphate and concentrated under vacuum to give the residue which was purified by combiflash column chromatography using 2-4% methanol in DCM as eluent to afford the title as light brown solid (1.8 g, 34%). ¹H NMR (400 MHz, DMSO-d₆): δ 8.09 (d, J=9.2 Hz, 1H), 7.40 (t, J=8.0 Hz, 1H), 7.29 (d, J=8.4 Hz, 1H), 7.11 (d, J=2.4 Hz 1H), 6.97 (dd, J=9.2, 1.6 Hz, 1H), 6.34 (d, J=8.4 Hz, 1H), 5.23-5.21 (m, 1H), 3.93 (s, 4H), 3.36 (t, J=5.6 Hz, 4H), 2.34-2.24 (m, 2H), 2.09-1.96 (m, 4H), 1.81-1.68 (m, 6H); LC-MS: m/z 460.3 (M+H).

### Step g: Synthesis of 4-amino-7-cyclopentyl-9-(4-oxopiperidin-1-yl)benzo[4,5]imidazo[1,2-a]quinazolin-5(7H)-one

To a stirred solution of 4-amino-7-cyclopentyl-9-(1,4-dioxa-8-azaspiro[4.5]decan-8-yl)benzo[4,5]imidazo[1,2-a]quinazolin-5(7H)-one (1.2 g, 2.611 mmol) in THF was added 6N HCl, stirred at 60° C. for 8 h. Reaction mixture was quenched with bicarbonate solution and extracted with 10% MeOH in DCM. The combined organic layers were dried over Na₂SO₄ and concentrated in vacuo to afford the title compound as light brown solid (1.0 g, 92%). ¹H NMR (400 MHz, DMSO-d6): δ 8.11 (d, J=8.8 Hz, 1H), 7.41 (t, J=8.0 Hz, 1H), 7.29 (d, J=8.4 Hz, 1H), 7.17 (d, J=2.4 Hz, 1H), 7.01 (d, J=2.4 Hz, 1H), 6.65 (d, J=6.8 Hz, 1H), 5.25-5.21 (m, 1H), 3.71 (t, J=6.0 Hz, 4H), 2.45 (t, J=5.8 Hz, 4H), 2.36-2.27 (m, 2H), 2.11-1.99 (m, 4H), 1.79-1.68 (m, 2H); LC-MS: m/z 416.2 (M+H).

The intermediates 6 to 13 listed in below Table-1 were prepared by procedure similar to the one described in synthesis of intermediate-5 with appropriate variations in reactants, quantities of reagents, protections and deprotections, solvents and reaction conditions. The characterization data of the compounds are summarized herein the below table.

TABLE 1

| Intermediate | Intermediate Structure & chemical | |
|---|---|---|
| No. | name | Characterization Data |
| 6 | 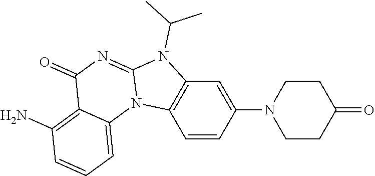 [SMILES: CC(C)n1c2cc(N3CCC(=O)CC3)ccc2n2c3cccc(N)c3c(=O)nc12] 4-amino-7-isopropyl-9-(4- oxopiperidin-1- yl)benzo[4,5]imidazo[1,2- a]quinazolin-5(7H)-one | ¹H NMR (400 MHz, DMSO-d6): δ 8.08 (d, J = 8.8 Hz, 1H), 7.40 (t, J = 8.4 Hz, 1H), 7.29 (d, J = 8.0 Hz, 1H), 7.25 (d, J = 2.0 Hz, 1H), 7.01 (dd, J = 9.2, 2.0 Hz, 1H), 6.64 (d, J = 8.0 Hz, 1H), 5.13-5.10 (m, 1H), 3.71 (t, J = 6.0 Hz, 4H), 2.45 (t, J = 5.8 Hz, 4H), 1.61 (d, J = 6.8 Hz, 6H). LC-MS: m/z 390.1 (M + H). |
| 7 | 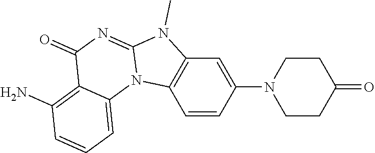 [SMILES: Cn1c2cc(N3CCC(=O)CC3)ccc2n2c3cccc(N)c3c(=O)nc12] 4-amino-7-methyl-9-(4- oxopiperidin-1- yl)benzo[4,5]imidazo[1,2- a]quinazolin-5(7H)-one | ¹H NMR (400 MHz, DMSO-d6): δ 8.05 (d, J = 8.8 Hz, 1H), 7.41 (t, J = 8.0 Hz, 1H), 7.27 (d, J = 8.4 Hz, 2H), 7.01 (d, J = 2.4 Hz, 1H), 6.65 (d, J = 6.8 Hz, 1H), 3.71 (t, J = 6.0 Hz, 4H), 3.63 (s, 3H), 2.45 (t, J = 5.8 Hz, 4H). LC-MS: m/z 362.1 (M + H). |
| 8 | 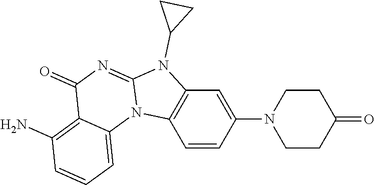 [SMILES: Nc1cccc2c1c(=O)nc1n(C3CC3)c3cc(N4CCC(=O)CC4)ccc3n21] 4-amino-7-cyclopropyl-9-(4- oxopiperidin-1- yl)benzo[4,5]imidazo[1,2- a]quinazolin-5(7H)-one | LC-MS: m/z 388.1 (M + H) |
| 9 | 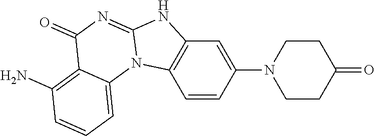 [SMILES: Nc1cccc2c1c(=O)nc1[nH]c3cc(N4CCC(=O)CC4)ccc3n12] 4-amino-9-(4-oxopiperidin-1- yl)benzo[4,5]imidazo[1,2- a]quinazolin-5(7H)-one | ¹H NMR (400 MHz, DMSO-d6): δ 8.05 (d, J = 8.8 Hz, 1H), 7.44 (t, J = 8.0 Hz, 1H), 7.27 (d, J = 8.4 Hz, 1H), 7.15 (d, J = 2.4 Hz, 1H), 7.01 (d, J = 6.8 Hz, 1H), 6.87 (d, J = 7.2 Hz, 1H), 6.64 (d, J = 8.4 Hz, 1H), 3.65 (t, J = 6.0 Hz, 4H), 2.45 (t, J = 5.8 Hz, 4H). LC-MS: m/z 348.1 (M + H). |
| 10 | 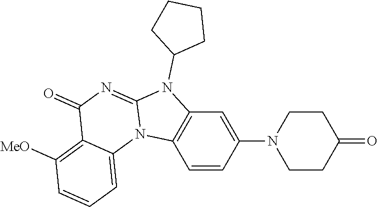 [SMILES: COc1cccc2c1c(=O)nc1n(C3CCCC3)c3cc(N4CCC(=O)CC4)ccc3n21] 7-cyclopentyl-4-methoxy-9-(4- oxopiperidin-1- yl)benzo[4,5]imidazo[1,2- a]quinazolin-5(7H)-one | ¹H NMR (400 MHz, DMSO-d6): δ 8.30 (d, J = 8.8 Hz, 1H), 7.60 (t, J = 8.0 Hz, 1H), 7.10-7.07 (m, 2H), 7.01 (dd, J = 8.8, 2.4 Hz, 1H), 6.80 (d, J = 7.2 Hz, 1H), 5.26-5.22 (m, 1H), 3.90 (s, 3H), 3.71 (t, J = 5.2 Hz, 4H), 2.45 (t, J = 5.8 Hz, 4H), 2.36-2.27 (m, 2H), 2.11-1.99 (m, 4H), 1.79-1.68 (m, 2H); LC-MS: m/z 431.2 (M + H). |
| 11 | 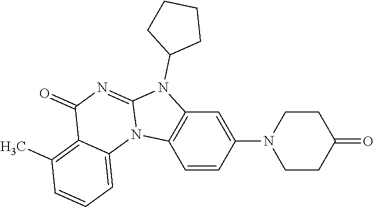 [SMILES: Cc1cccc2c1c(=O)nc1n(C3CCCC3)c3cc(N4CCC(=O)CC4)ccc3n21] 7-cyclopentyl-4-methyl-9-(4- oxopiperidin-1- yl)benzo[4,5]imidazo[1,2- a]quinazolin-5(7H)-one | ¹H NMR (400 MHz, DMSO-d6): δ 8.30 (d, J = 8.8 Hz, 1H), 7.56 (t, J = 8.0 Hz, 1H), 7.40 (d, J = 8.0 Hz, 1H), 7.13 (d, J = 6.8 Hz, 1H), 7.07 (d, J = 4.0 Hz, 1H), 7.01 (dd, J = 8.8, 2.4 Hz, 1H), 5.26-5.22 (m, 1H), 3.67 (t, J = 5.2 Hz, 4H), 2.99 (s, 3H), 2.45 (t, J = 5.8 Hz, 4H), 2.36-2.27 (m, 2H), 2.11-1.99 (m, 4H), 1.79-1.68 (m, 2H); LC-MS: m/z 415.2 (M + H). |
| 12 | 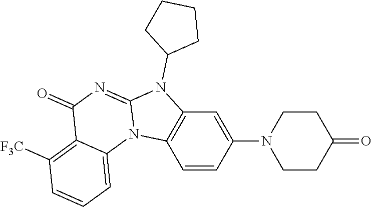 [SMILES: O=C1CCN(c2ccc3c(c2)n(C2CCCC2)c2nc(=O)c4c(C(F)(F)F)cccc4n32)CC1] 7-cyclopentyl-9-(4-oxopiperidin-1- yl)-4- (trifluoromethyl)benzo[4,5]imidazo[ 1,2-a]quinazolin-5(7H)-one | ¹H NMR (400 MHz, DMSO-d6): δ 8.39 (d, J = 8.8 Hz, 1H), 7.89-7.86 (m, 2H), 7.74 (d, J = 4.0 Hz, 1H), 7.21 (d, J = 2.4 Hz, 1H), 7.05 (dd, J = 8.8, 2.4 Hz, 1H), 5.36-5.30 (m, 1H), 3.69 (t, J = 5.2 Hz, 4H), 2.46 (t, J = 5.8 Hz, 4H), 2.36-2.27 (m, 2H), 2.11-1.99 (m, 4H), 1.79-1.68 (m, 2H); LC-MS: m/z 469.1 (M + H). |
| 13 | 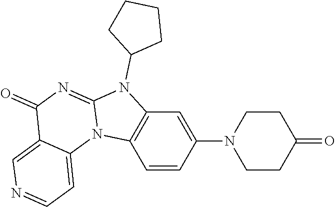 [SMILES: O=C1CCN(c2ccc3c(c2)n(C2CCCC2)c2nc(=O)c4cnccc4n32)CC1] 7-cyclopentyl-9-(4-oxopiperidin-1- yl)benzo[4,5]imidazo[1,2- a]pyrido[3,4-e]pyrimidin-5(7H)-one | ¹H NMR (400 MHz, DMSO-d6): δ 9.30 (s, 1H), 8.70 (s, 1H), 8.30 (d, J = 8.8 Hz, 1H), 7.45 (d, J = 7.0 Hz, 1H), 7.21 (s, 1H), 7.08 (dd, J = 8.8, 2.4 Hz, 1H), 5.36-5.30 (m, 1H), 3.70 (t, J = 5.2 Hz, 4H), 2.46 (t, J = 5.8 Hz, 4H), 2.36-2.27 (m, 2H), 2.11-1.99 (m, 4H), 1.79-1.68 (m, 2H); LC-MS: m/z 402.1 (M + H). |
| 14 | 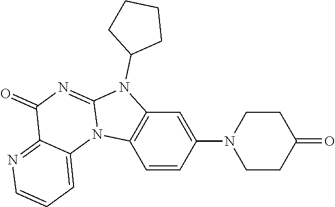 [SMILES: O=C1CCN(c2ccc3c(c2)n(C2CCCC2)c2nc(=O)c4ncccc4n32)CC1] 7-cyclopentyl-9-(4-oxopiperidin-1- yl)benzo[4,5]imidazo[1,2- a]pyrido[2,3-e]pyrimidin-5(7H)-one | ¹H NMR (400 MHz, DMSO-d6): δ 8.87 (d, J = 8.0 Hz, 1H), 8.78 (s, 1H), 8.25 (d, J = 8.0 Hz, 1H), 7.85 (s, 1H), 7.23 (s, 1H), 7.08 (dd, J = 8.8, 2.4 Hz, 1H), 5.36-5.30 (m, 1H), 3.69 (t, J = 5.2 Hz, 4H), 2.46 (t, J = 5.8 Hz, 4H), 2.36-2.27 (m, 2H), 2.11-1.99 (m, 4H), 1.79-1.68 (m, 2H); LC-MS: m/z 402.1 (M + H). |
| 15 | 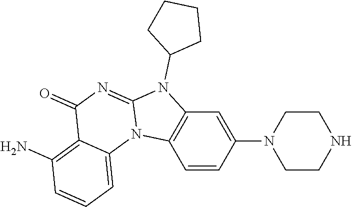 [SMILES: Nc1cccc2c1c(=O)nc1n(C3CCCC3)c3cc(N4CCNCC4)ccc3n21] 4-amino-7-cyclopentyl-9-(piperazin- 1-yl)benzo[4,5]imidazo[1,2- a]quinazolin-5(7H)-one | LC-MS: m/z 403.2 (M + H). |
| 16 | 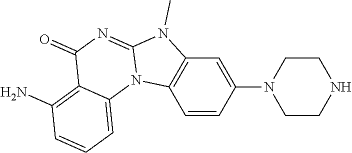 [SMILES: Cn1c2cc(N3CCNCC3)ccc2n2c3cccc(N)c3c(=O)nc12] 4-amino-7-methyl-9-(piperazin-1- yl)benzo[4,5]imidazo[1,2- a]quinazolin-5(7H)-one | LC-MS: m/z 349.1 (M + H). |
| 17 | 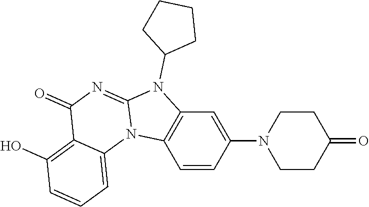 [SMILES: O=C1CCN(c2ccc3c(c2)n(C2CCCC2)c2nc(=O)c4c(O)cccc4n32)CC1] 7-cyclopentyl-4-hydroxy-9-(4- oxopiperidin-1- yl)benzo[4,5]imidazo[1,2- a]quinazolin-5(7H)-one | ¹H NMR (400 MHz, DMSO-d6): δ 8.26 (d, J = 8.8 Hz, 1H), 7.60 (t, J = 8.0 Hz, 1H), 7.15 (d, J = 2.4 Hz, 1H), 7.04-7.1 (m, 2H), 6.66 (dd, J = 8.8, 2.4 Hz, 1H), 5.26-5.22 (m, 1H), 3.71 (t, J = 5.2 Hz, 4H), 2.45 (t, J = 5.8 Hz, 4H), 2.36-2.27 (m, 2H), 2.11-1.99 (m, 4H), 1.79-1.68 (m, 2H); LC-MS: m/z 417.1 (M + H). |

### Intermediate-18: 4-Chloro-7-cyclopentyl-9-(4-oxopiperidin-1-yl)benzo[4,5]imidazo[1,2-a]quinazolin-5(7H)-one

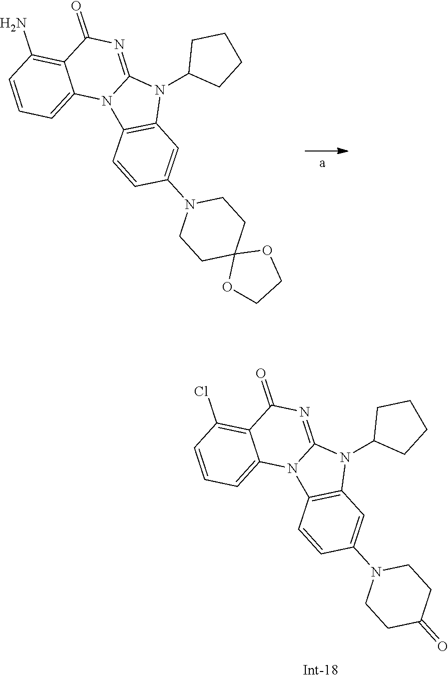 [SMILES: Nc1cccc2c1c(=O)nc1n(C3CCCC3)c3cc(N4CCC5(CC4)OCCO5)ccc3n21 ; O=C1CCN(c2ccc3c(c2)n(C2CCCC2)c2nc(=O)c4c(Cl)cccc4n32)CC1]

### Step a: Synthesis of 4-chloro-7-cyclopentyl-9-(4-oxopiperidin-1-yl)benzo[4,5]imidazo[1,2-a]quinazolin-5(7H)-one

To a stirred solution of 4-amino-7-cyclopentyl-9-(1,4-dioxa-8-azaspiro[4.5]decan-8-yl)benzo[4,5]imidazo[1,2-a]quinazolin-5(7H)-one (1.5 g, 3.26 mmol) in acetonitrile (40 mL) and acetic acid (9 mL) was added sodium nitrite (0.22 g, 3.26 mmol) dissolved in water (2 mL) dropwise at 0° C. and stirred for 15 minutes. Then copper (I) chloride (0.35 g, 3.59 mmol) was added portion wise to the reaction mixture followed by conc. HCl was added dropwise at the at 0° C. The mixture was allowed to reach RT and then stirred at 50° C. for 30 minutes. Then the reaction mixture was diluted with water and basified with sodium bicarbonate solution to pH 8 and extracted with DCM:MeOH (9:1) three times. The combined organic layers were dried over Na₂SO₄ and concentrated in vacuo to get crude compound which was purified by combiflash column chromatography using 3-4% methanol in DCM as eluent to afford mixture of ketal and keto compound. Mixture of ketal and keto compounds were taken in THF and added 6N HCl, stirred at 60° C. for 8 h. Reaction mixture was quenched with bicarbonate solution and extracted with 10% MeOH in DCM. The combined organic layers were dried over Na₂SO₄ and concentrated in vacuo to afford the title compound as light brown solid (0.5 g, 31.9%). ¹H NMR (400 MHz, DMSO-d₆): δ 8.37 (d, J=8.4 Hz, 1H), 8.22 (d, J=9.2 Hz, 1H), 7.77 (t, J=8.0 Hz, 1H), 7.55 (d, J=7.2 Hz, 1H), 7.20 (d, J=2.4H), 7.05 (dd, J=9.2, 2.4 Hz, 1H), 5.27-5.19 (m, 1H), 3.72 (t, J=6.0 Hz, 4H), 2.49 (t, J=6.0 Hz, 4H), 2.38-2.28 (m, 2H), 2.08-1.98 (m, 4H), 1.80-1.69 (m, 2H); LC-MS: m/z 435.2 (M+H).

The intermediates 19 to 24 listed in below Table-2 were prepared by procedure similar to the one described in synthesis of intermediate-18 with appropriate variations in reactants, quantities of reagents, protections and deprotections, solvents and reaction conditions. The characterization data of the compounds are summarized herein the below table.

TABLE-2

| Int. | Intermediate Structure & chemical | |
|---|---|---|
| No. | name | Characterization Data |
| 19 | 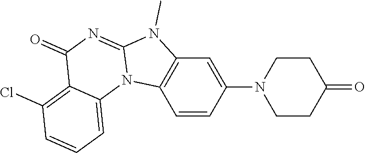 [SMILES: Cn1c2cc(N3CCC(=O)CC3)ccc2n2c3cccc(Cl)c3c(=O)nc12] | ¹H NMR (400 MHz, DMSO-d6): δ 8.34 (d, J = 8.8 Hz, 1H), 8.20 (d, J = 8.0 Hz, 1H), 7.80 (t, J = 8.4 Hz, 1H), 7.60 (d, J = 8.0 Hz, 1H), 7.30 (s, 1H), 7.01 (dd, J = 9.2, 2.0 Hz, 1H), 3.70 (t, J = 6.0 Hz, 1H), 3.61 (s, 3H), 2.45 (t, J = 5.8 Hz, 4H). LC-MS: m/z 381.1 (M + H). |
| | 4-chloro-7-methyl-9-(4- | |
| | oxopiperidin-1- | |
| | yl)benzo[4,5]imidazo[1,2- | |
| | a]quinazolin-5(7H)-one | |
| 20 | 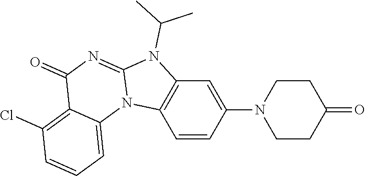 [SMILES: CC(C)n1c2cc(N3CCC(=O)CC3)ccc2n2c3cccc(Cl)c3c(=O)nc12] | ¹H NMR (400 MHz, DMSO-d6): δ 8.33 (d, J = 8.8 Hz, 1H), 8.20 (d, J = 8.0 Hz, 1H), 7.80 (t, J = 8.4 Hz, 1H), 7.60 (d, J = 8.0 Hz, 1H), 7.30 (s, 1H), 7.01 (dd, J = 9.2, 2.0 Hz, 1H), 5.13-5.10 (m, 1H), 3.71 (t, J = 6.0 Hz, 4H), 2.45 (t, J = 5.8 Hz, 4H), 1.61 (d, J = 6.8 Hz, 6H). LC-MS: m/z 409.1 (M + H). |
| | 4-chloro-7-isopropyl-9-(4- | |
| | oxopiperidin-1- | |
| | yl)benzo[4,5]imidazo[1,2- | |
| | a]quinazolin-5(7H)-one | |
| 21 | 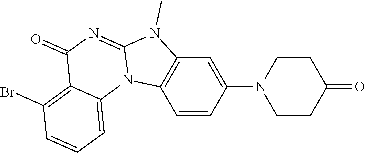 [SMILES: Cn1c2cc(N3CCC(=O)CC3)ccc2n2c3cccc(Br)c3c(=O)nc12] | ¹H NMR (400 MHz, DMSO-d6): δ 8.41 (d, J = 8.8 Hz, 1H), 8.21 (d, J = 8.0 Hz, 1H), 7.77 (d, J = 8.4 Hz, 1H), 7.66 (t, J = 8.0 Hz, 1H), 7.21 (d, J = 2.4 Hz, 1H), 7.04 (dd, J = 9.2, 2.0 Hz, 1H), 3.71 (t, J = 6.0 Hz, 4H), 3.61 (s, 3H), 2.45 (t, J = 5.8 Hz, 4H). LC- MS: m/z 425.0 (M + H). |
| | 4-bromo-7-methyl-9-(4- | |
| | oxopiperidin-1- | |
| | yl)benzo[4,5]imidazo[1,2- | |
| | a]quinazolin-5(7H)-one | |
| 22 | 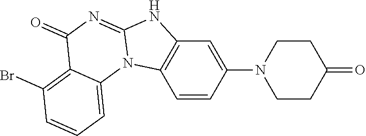 [SMILES: O=C1CCN(c2ccc3c(c2)[nH]c2nc(=O)c4c(Br)cccc4n23)CC1] | LC-MS: m/z 411.0 (M + H). |
| | 4-bromo-9-(4-oxopiperidin-1- | |
| | yl)benzo[4,5]imidazo[1,2- | |
| | a]quinazolin-5(7H)-one | |
| 23 | 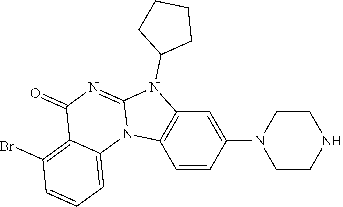 [SMILES: O=c1nc2n(C3CCCC3)c3cc(N4CCNCC4)ccc3n2c2cccc(Br)c12] | LC-MS: m/z 466.1 (M + H). |
| | 4-bromo-7-cyclopentyl-9-(piperazin- | |
| | 1-yl)benzo[4,5]imidazo[1,2- | |
| | a]quinazolin-5(7H)-one | |
| 24 | 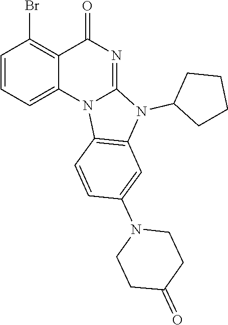 [SMILES: O=C1CCN(c2ccc3c(c2)n(C2CCCC2)c2nc(=O)c4c(Br)cccc4n32)CC1] | ¹H NMR (400 MHz, DMSO-d₆): δ 8.41 (d, J = 8.0 Hz, 1H), 8.21 (d, J = 9.2 Hz, 1H), 7.77 (d, J = 7.2 Hz, 1H), 7.66 (t, J = 8.0 Hz, 1H), 7.20 (d, J = 2.4 Hz. 1H), 7.04 (dd, J = 9.2, 1.6 Hz, 1H), 5.28-5.20 (m, 1H), 3.71 (t, J = 6.0 Hz, 4H), 2.49 (t, J = 6.0 Hz, 4H), 2.37-2.27 (m, 2H), 2.09-1.98 (m, 4H), 1.79- 1.68 (m, 2H); LC-MS: m/z 479.2 (M + H). |

### Intermediate-25: Synthesis of 7-Cyclopentyl-9-(4-oxopiperidin-1-yl)benzo[4,5]imidazo[1,2-a]quinazolin-5(7H)-one

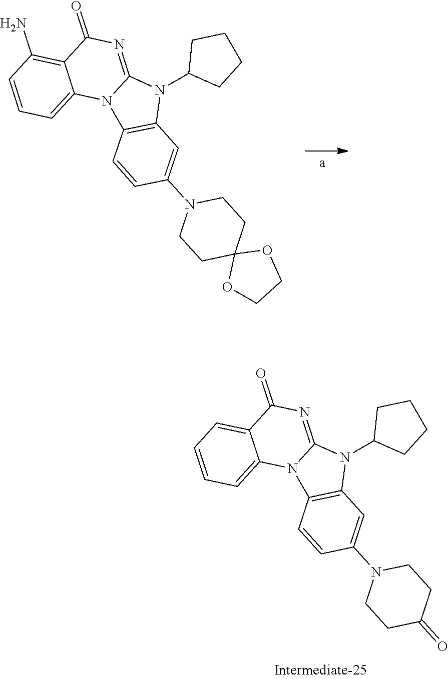 [SMILES: Nc1cccc2c1c(=O)nc1n(C3CCCC3)c3cc(N4CCC5(CC4)OCCO5)ccc3n21 ; O=C1CCN(c2ccc3c(c2)n(C2CCCC2)c2nc(=O)c4ccccc4n32)CC1]

### Step a: Synthesis of 7-cyclopentyl-9-(4-oxopiperidin-1-yl)benzo[4,5]imidazo[1,2-a]quinazolin-5(7H)-one

To a stirred solution of 4-amino-7-cyclopentyl-9-(1,4-dioxa-8-azaspiro[4.5]decan-8-yl)benzo[4,5]imidazo[1,2-a]quinazolin-5(7H)-one (0.08 g, 0.174 mmol) in acetonitrile (2 mL) and acetic acid (0.8 mL) was added sodium nitrite (0.012 g, 0.172 mmol) dissolved in water (0.5 mL) dropwise at 0° C. and stirred for 15 minutes. The mixture was allowed to reach RT and then stirred at 70° C. for 20 minutes. Then the reaction mixture was quenched with bicarbonate solution and extracted with 10% MeOH in DCM. The combined organic layers were dried over Na₂SO₄ and concentrated in vacuo to get afford mixture of ketal and keto compound. Mixture of ketal and keto compound was taken in THF, 6N HCl was added and stirred at 60° C. for 6 h. Reaction mixture was quenched with bicarbonate solution and extracted with 10% MeOH in DCM. The combined organic layer was taken for purification by combiflash column chromatography using 1-3% methanol in DCM as eluent to afford the title compound as yellow solid (0.040 g, 51%). ¹H NMR (400 MHz, DMSO-d₆): δ 8.42 (d, J=8.4 Hz, 1H), 8.28-8.22 (m, 2H), 7.87 (dt, J=8.4, 1.6 Hz, 1H), 7.54 (t, J=7.6 Hz, 1H), 7.22 (d, J=2.4H), 7.08 (dd, J=8.8, 2.4 Hz, 1H), 5.32-5.22 (m, 1H), 3.72 (t, J=6.4 Hz, 4H), 2.51 (t, J=6.4 Hz, 4H), 2.40-2.30 (m, 2H), 2.11-1.98 (m, 4H), 1.81-1.71 (m, 2H); LC-MS: m/z 401.2 (M+H).

### Example-1: 3-(5-(1′-(4-Bromo-7-cyclopentyl-5-oxo-5,7-dihydrobenzo[4,5]imidazo[1,2-a]quinazolin-9-yl)-[1,4′-bipiperidin]-4-yl)-1-oxoisoindolin-2-yl)piperidine-2,6-dione (Compound 1)

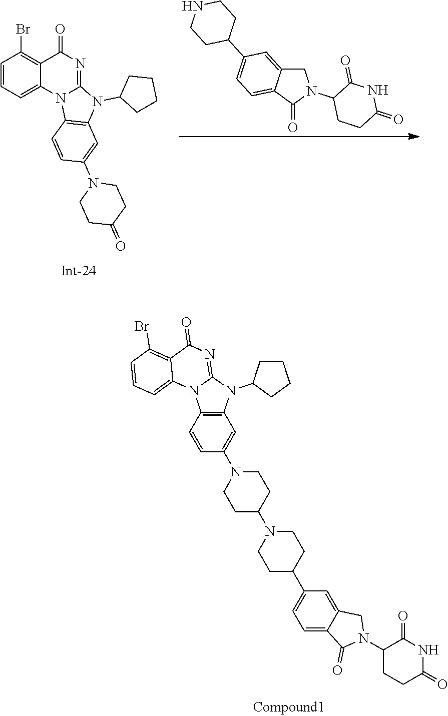 [SMILES: O=C1CCN(c2ccc3c(c2)n(C2CCCC2)c2nc(=O)c4c(Br)cccc4n32)CC1 ; O=C1CCC(N2Cc3cc(C4CCNCC4)ccc3C2=O)C(=O)N1 ; O=C1CCC(N2Cc3cc(C4CCN(C5CCN(c6ccc7c(c6)n(C6CCCC6)c6nc(=O)c8c(Br)cccc8n76)CC5)CC4)ccc3C2=O)C(=O)N1]

4-bromo-7-cyclopentyl-9-(4-oxopiperidin-1-yl)benzo[4,5]imidazo[1,2-a]quinazolin-5(7H)-one (0.095 g, 0.198 mmol) and 3-(1-oxo-5-(piperidin-4-yl) isoindolin-2-yl)piperidine-2,6-dione (0.065 g, 0.198 mmol) were added in methanol (5 mL) to obtain a solution. To the solution, zinc chloride in THF (1M) (0.2 mL, 0.198 mmol) was added dropwise at 0° C. and stirred for 15 mins to obtain a first reaction mixture. Sodium cyanoborohydride (0.027 g, 0.43 mmol) was added to the first reaction mixture at 0° C. and then stirred for 24 h at RT to obtain a second reaction mixture. The second reaction mixture was poured into ice cold water and extracted with 10% methanol in DCM. The combined organic layer was washed with water and brine, dried over anhydrous sodium sulphate and concentrated under vacuum to afford the crude product which was purified by combi flash column chromatography using 8% methanol in DCM as eluent to afford the title compound as light yellow solid (0.018 g, 11%). ¹H NMR (400 MHz, DMSO-d₆): δ 10.96 (s, 1H), 8.43 (d, J=8.4 Hz, 1H), 8.19 (d, J=8.4 Hz, 1H), 7.77 (t, J=8.8 Hz, 1H), 7.67-7.64 (m, 2H), 7.50 (s, 1H), 7.39 (d, J=8.4 Hz, 1H), 7.13 (d, J=2.0 Hz, 1H), 6.98 (dd, J=8.0, 1.6 Hz, 1H), 5.23 (t, J=8.0 Hz, 1H), 5.10 (dd, J=13.2, 4.8 Hz, 1H), 4.43 (d, J=17.2 Hz, 1H), 4.29 (d, J=17.2 Hz, 1H), 3.88 (d, J=11.6 Hz, 2H), 3.08-2.98 (m, 2H), 2.97-2.76 (m, 3H), 2.66-2.57 (m, 2H), 2.43-2.26 (m, 3H), 2.08-1.95 (m, 7H), 1.95-1.62 (m, 11H); LC-MS: m/z 790.5 (M−H).

The compounds listed in below Table-3 were prepared by procedure similar to the one described in Example-I with appropriate variations in reactants, quantities of reagents, protections and deprotections, solvents and reaction conditions. The characterization data of the compounds are summarized herein the below table.

TABLE-3

| Comp. | | | |
|---|---|---|---|
| No. | Intermediate | Structure | Characterization Data |
| 2 | Int-24 & Int-2 | 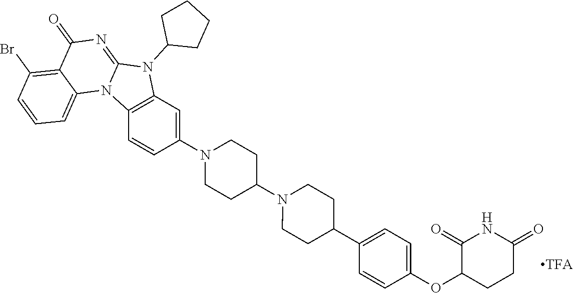 [SMILES: O=C1CCC(Oc2ccc(C3CCN(C4CCN(c5ccc6c(c5)n(C5CCCC5)c5nc(=O)c7c(Br)cccc7n65)CC4)CC3)cc2)C(=O)N1] | ¹H NMR (400 MHz, DMSO- d₆): δ 10.92 (s, 1H), 9.14 (bs, 1H), 8.42 (d, J = 8.8 Hz, 1H), 8.22 (d, J = 8.8 Hz, 1H), 7.79 (d, J = 8.0 Hz, 1H), 7.67 (d, J = 8.0 Hz, 1H), 7.22-7.14 (m, 3H), 7.06-6.98 (m, 3H), 5.27-5.15 (m, 2H), 4.05-3.98 (m, 2H), 3.63 (d, J = 11.2 Hz, 2H), 3.21- 3.11 (m, 2H), 2.92-2.80 (m, 2H), 2.78-2.2.60 (m, 2H), 2.37- 2.30 (m, 2H), 2.26-1.96 (m, 11H), 1.95-1.74 (m, 7H); LC- MS: m/z 751.5 (M − H). |
| | | 3-(4-(1′-(4-bromo-7-cyclopentyl- | |
| | | 5-oxo-5,7- | |
| | | dihydrobenzo[4,5]imidazo[1,2- | |
| | | a]quinazolin-9-yl)-[1,4′- | |
| | | bipiperidin]-4- | |
| | | yl)phenoxy)piperidine-2,6- | |
| | | dione-TFA salt | |
| 3 | Int-24 & Int-3 | 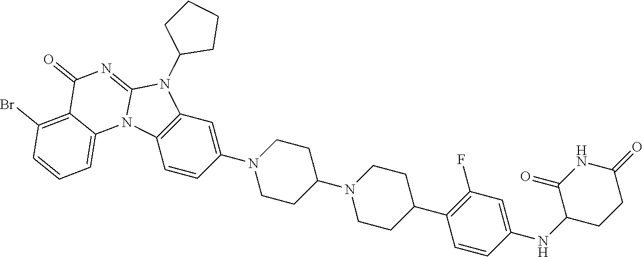 [SMILES: O=C1CCC(Nc2ccc(C3CCN(C4CCN(c5ccc6c(c5)n(C5CCCC5)c5nc(=O)c7c(Br)cccc7n65)CC4)CC3)c(F)c2)C(=O)N1] | ¹H NMR (400 MHz, DMSO- d₆): δ 10.78 (s, 1H), 8.41 (d, J = 8.8 Hz, 1H), 8.18 (d, J = 8.8 Hz, 1H), 7.77 (d, J = 8.4 Hz, 1H), 7.66 (d, J = 8.8 Hz, 1H), 7.13 (d, J = 2.0 Hz, 1H), 7.02-6.98 (m, 2H), 6.46-6.42 (m, 2H), 5.99 (d, J = 8.0 Hz, 1H), 5.23 (t, J = 8.4 Hz, 1H), 4.34-4.26 (m, 1H), 3.87 (d, J = 11.6 Hz, 2H), 3.03- 2.96 (m, 2H), 2.82-2.55 (m, 4H), 2.31-2.23 (m, 4H), 2.14- 1.96 (m, 6H), 1.95-1.80 (m, 3H), 1.80-1.54 (m, 9H); LC- MS: m/z 766.2 (M − H). |
| | | 3-((4-(1′-(4-bromo-7- | |
| | | cyclopentyl-5-oxo-5,7- | |
| | | dihydrobenzo[4,5]imidazo[1,2- | |
| | | a]quinazolin-9-yl)-[1,4′- | |
| | | bipiperidin]-4-yl)-3- | |
| | | fluorophenyl)amino)piperidine- | |
| | | 2,6-dione | |
| 4 | Int-24 & 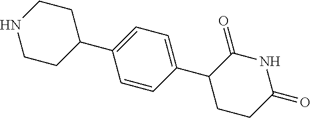 [SMILES: O=C1CCC(c2ccc(C3CCNCC3)cc2)C(=O)N1] | 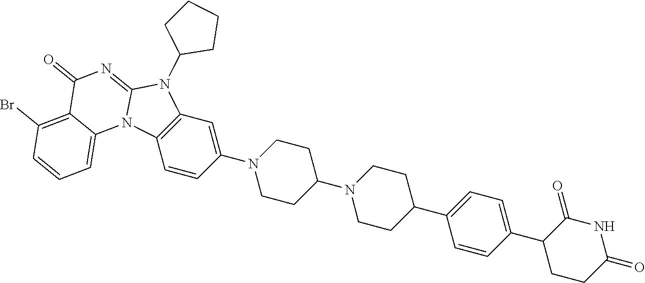 [SMILES: O=C1CCC(c2ccc(C3CCN(C4CCN(c5ccc6c(c5)n(C5CCCC5)c5nc(=O)c7c(Br)cccc7n65)CC4)CC3)cc2)C(=O)N1] | ¹H NMR (400 MHz, DMSO- d₆): δ 10.82 (s, 1H), 8.41 (d, J = 8.0 Hz, 1H), 8.19 (d, J = 9.2 Hz, 1H), 7.78 (d, J = 7.2 Hz, 1H), 7.68 (d, J = 8.0 Hz, 1H), 7.22-7.13 (m, 5H), 6.98 (dd, J = 8.0, 1.6 Hz, 1H), 5.24 (t, J = 8.0 Hz, 1H), 3.86 (d, J = 11.2 Hz, 1H), 3.85-3.77 (m, 1H), 3.07-2.98 (m, 1H), 2.81-2.75 (m, 2H), 2.62-2.56 (m, 2H), 2.36-2.05 (m, 5H), 2.04-1.86 (m, 9H), 1.82-1.58 (m, 8H); LC-MS: m/z 735.2 (M + H). |
| | | 3-(4-(1′-(4-bromo-7-cyclopentyl- | |
| | | 5-oxo-5,7- | |
| | | dihydrobenzo[4,5]imidazo[1,2- | |
| | | a]quinazolin-9-yl)-[1,4′- | |
| | | bipiperidin]-4- | |
| | | yl)phenyl)piperidine-2,6-dione | |
| 5 | Int-23 & 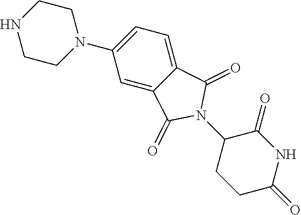 [SMILES: O=C1CCC(N2C(=O)c3ccc(N4CCNCC4)cc3C2=O)C(=O)N1] | 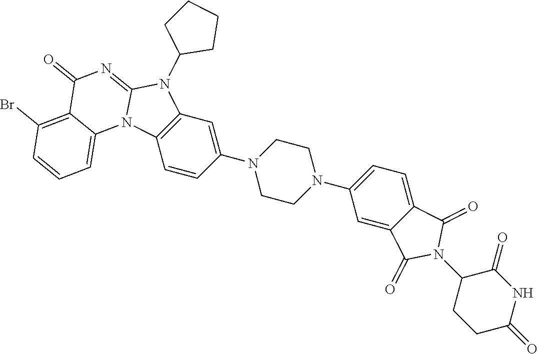 [SMILES: O=C1CCC(N2C(=O)c3ccc(N4CCN(c5ccc6c(c5)n(C5CCCC5)c5nc(=O)c7c(Br)cccc7n65)CC4)cc3C2=O)C(=O)N1] | ¹H NMR (400 MHz, DMSO- d₆): δ 11.11 (s, 1H), 8.44 (d, J = 8.0 Hz, 1H), 8.25 (d, J = 9.2 Hz, 1H), 7.78 (d, J = 8.0 Hz, 1H), 7.74 (d, J = 8.0 Hz, 1H), 7.67 (t, J = 8.0 Hz, 1H), 7.45 (d, J = 1.6 Hz, 1H), 7.36 (dd, J = 8.8, 2.0 Hz, 1H), 7.20 (d, J = 2.0 Hz, 1H), 7.05 (dd, J = 9.2, 1.6 Hz, 1H), 5.25 (t, J = 8.8 Hz, 1H), 5.11-5.07 (m, 1H), 3.71 (bs, 4H), 3.45 (m, 4H), 2.62-2.52 (m, 4H), 2.09-1.96 (m, 6H), 1.81-1.70 (m, 2H); LC-MS: m/z 722.3 (M − H). |
| | | 5-(4-(4-bromo-7-cyclopentyl-5- | |
| | | oxo-5,7- | |
| | | dihydrobenzo[4,5]imidazo[1,2- | |
| | | a]quinazolin-9-yl)piperazin-1- | |
| | | yl)-2-(2,6-dioxopiperidin-3- | |
| | | yl)isoindoline-1,3-dione | |
| 6 | Int-24 & Int-1 | 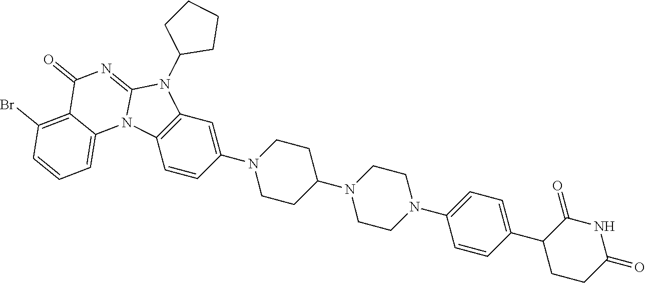 [SMILES: O=C1CCC(c2ccc(N3CCN(C4CCN(c5ccc6c(c5)n(C5CCCC5)c5nc(=O)c7c(Br)cccc7n65)CC4)CC3)cc2)C(=O)N1] | ¹H NMR (400 MHz, DMSO- d₆): δ 10.77 (s, 1H), 8.41 (d, J = 8.4 Hz, 1H), 8.19 (d, J = 9.2 Hz, 1H), 7.77 (d, J = 7.6 Hz, 1H), 7.68 (t, J = 8.0 Hz, 1H), 7.13 (d, J = 2.4 Hz, 1H), 7.05 (d, J = 8.4 Hz, 2H), 7.00 (dd, J = 9.2, 2.0 Hz, 1H), 6.90 (d, J = 8.4 Hz, 2H), 5.23 (t, J = 8.0 Hz, 1H), 3.87 (d, J = 12 Hz, 2H), 3.75- 3.71 (m, 1H), 3.13 (bs, 4H), 2.86-2.77 (m, 2H), 2.71-2.57 (m, 6H), 2.25-2.06 (m, 4H), 2.06-1.91 (m, 8H), 1.80-1.55 (m, 4H); LC-MS: m/z 736.5 (M + H). |
| | | 3-(4-(4-(1-(4-bromo-7- | |
| | | cyclopentyl-5-oxo-5,7- | |
| | | dihydrobenzo[4,5]imidazo[1,2- | |
| | | a]quinazolin-9-yl)piperidin-4- | |
| | | yl)piperazin-1- | |
| | | yl)phenyl)piperidine-2,6-dione | |
| 7 | Int-24 & 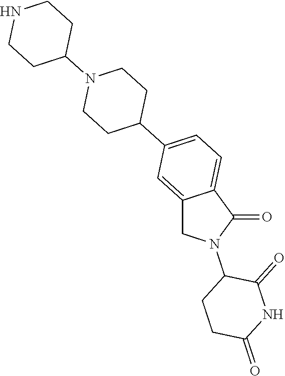 [SMILES: O=C1CCC(N2Cc3cc(C4CCN(C5CCNCC5)CC4)ccc3C2=O)C(=O)N1] | 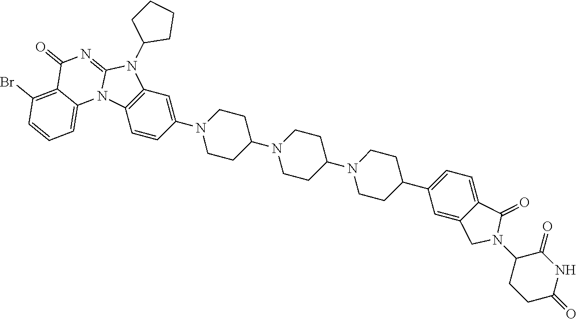 [SMILES: O=C1CCC(N2Cc3cc(C4CCN(C5CCN(C6CCN(c7ccc8c(c7)n(C7CCCC7)c7nc(=O)c9c(Br)cccc9n87)CC6)CC5)CC4)ccc3C2=O)C(=O)N1] | ¹H NMR (400 MHz, DMSO- d₆): δ 11.01 (s, 1H), 8.41 (d, J = 8.4 Hz, 1H), 8.18 (d, J = 9.2 Hz, 1H), 7.77 (d, J = 7.6 Hz, 1H), 7.68-7.63 (m, 2H), 7.48 (s, 1H), 7.39 (d, J = 8.0 Hz, 1H), 7.12 (d, J = 2.0 Hz, 1H), 6.98 (dd, J = 9.2, 1.6 Hz, 1H), 5.23 (t, J = 8.8 Hz, 1H), 5.11 (dd, J = 13.2, 4.8 Hz, 1H), 4.42 (d, J = 17.2 Hz, 1H), 4.28 (d, J = 17.2 Hz, 1H), 3.84 (dd, J = 12.4, 2.0 Hz, 2H), 3.05-2.85 (m, 5H), 2.83-2.72 (m, 2H), 2.64-2.55 (m, 2H), 2.42-2.10 (m, 8H), 2.08-1.95 (m, 5H), 1.92-1.37 (m, 15H); LC-MS: m/z 873.3 (M − H). |
| | | 3-(5-(1″-(4-bromo-7- | |
| | | cyclopentyl-5-oxo-5,7- | |
| | | dihydrobenzo[4,5]imidazo[1,2- | |
| | | a]quinazolin-9-yl)-[1,4′:1′,4″- | |
| | | terpiperidin]-4-yl)-1- | |
| | | oxoisoindolin-2-yl)piperidine- | |
| | | 2,6-dione | |
| 8 | Int-5 & 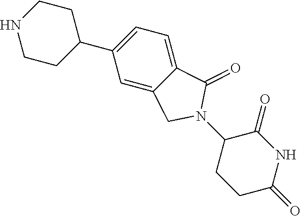 [SMILES: O=C1CCC(N2Cc3cc(C4CCNCC4)ccc3C2=O)C(=O)N1] | 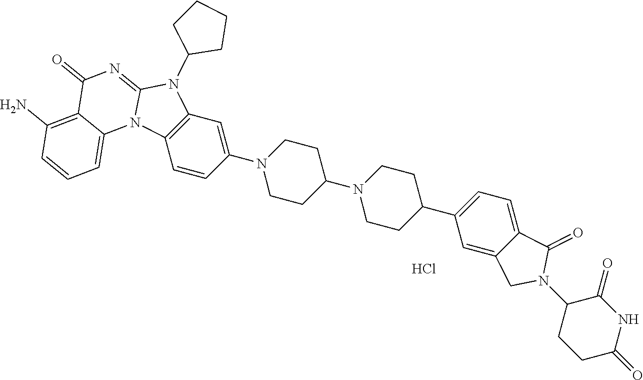 [SMILES: Nc1cccc2c1c(=O)nc1n(C3CCCC3)c3cc(N4CCC(N5CCC(c6ccc7c(c6)CN(C6CCC(=O)NC6=O)C7=O)CC5)CC4)ccc3n21 ; Cl] | ¹H NMR (400 MHz, DMSO- d₆): δ 11.01 (s, 1H), 10.52 (bs, 1H), 8.16 (d, J = 8.8 Hz, 1H), 7.73 (d, J = 8.0 Hz, 1H), 7.49 (bs, 1H), 7.43 (d, J = 8.4 Hz, 2H), 7.33 (d, J = 8.0 Hz, 1H), 7.16 (d, J = 2.0 Hz, 1H), 7.06 (d, J = 9.2, Hz, 1H), 6.68 (d, J = 8.0 Hz, 1H), 5.24 (t, J = 8.8 Hz, 1H), 5.12 (dd, J = 13.2, 4.8 Hz, 1H), 4.47 (d, J = 17.2 Hz, 1H), 4.33 (d, J = 17.2 Hz, 1H), 4.01 (d, J = 13.6 Hz, 2H), 3.22-2.81 (m, 5H), 2.64-2.55 (m, 2H), 2.41-2.12 (m, 6H), 2.10-1.84 (m, 13H), 1.81-1.67 (m, 2H); LC-MS: m/z 727.5 (M − H). |
| | | 3-(5-(1′-(4-amino-7-cyclopentyl- | |
| | | 5-oxo-5,7- | |
| | | dihydrobenzo[4,5]imidazo[1,2- | |
| | | a]quinazolin-9-yl)-[1,4′- | |
| | | bipiperidin]-4-yl)-1- | |
| | | oxoisoindolin-2-yl)piperidine- | |
| | | 2,6-dione-HCl salt | |
| 9 | Int-18 & 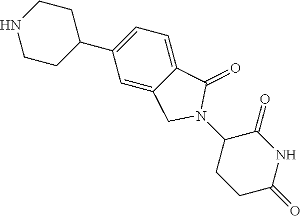 [SMILES: O=C1CCC(N2Cc3cc(C4CCNCC4)ccc3C2=O)C(=O)N1] | 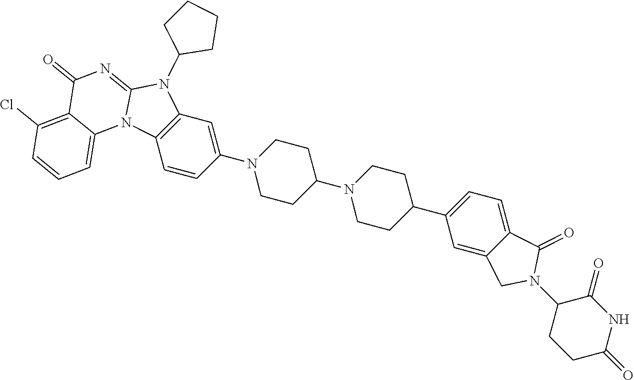 [SMILES: O=C1CCC(N2Cc3cc(C4CCN(C5CCN(c6ccc7c(c6)n(C6CCCC6)c6nc(=O)c8c(Cl)cccc8n76)CC5)CC4)ccc3C2=O)C(=O)N1] | ¹H NMR (400 MHz, DMSO- d₆): δ 10.98 (s, 1H), 8.36 (d, J = 8.8 Hz, 1H), 8.19 (d, J = 8.4 Hz, 1H), 7.76 (t, J = 8.0 Hz, 1H), 7.65 (d, J = 8.0 Hz, 1H), 7.55 (d, J = 7.2 Hz, 1H), 7.50 (s, 1H), 7.41 (d, J = 8.0 Hz, 1H), 7.13 (d, J = 2.0 Hz, 1H), 6.98 (dd, J = 9.2, 2.0 Hz, 1H), 5.23 (t, J = 8.0 Hz, 1H), 5.10 (dd, J = 13.2, 4.8 Hz, 1H), 4.43 (d, J = 17.2 Hz, 1H), 4.29 (d, J = 17.2 Hz, 1H), 3.88 (d, J = 12.0 Hz, 2H), 3.04 (d, J = 10.8 Hz, 2H), 2.97-2.75 (m, 3H), 2.68-2.57 (m, 2H), 2.45-2.25 (m, 5H), 2.10-1.86 (m, 8H), 1.86-1.60 (m, 8H); LC-MS: m/z 746.5 (M − H). |
| | | 3-(5-(1′-(4-chloro-7-cyclopentyl- | |
| | | 5-oxo-5,7- | |
| | | dihydrobenzo[4,5]imidazo[1,2- | |
| | | a]quinazolin-9-yl)-[1,4′- | |
| | | bipiperidin]-4-yl)-1- | |
| | | oxoisoindolin-2-yl)piperidine- | |
| | | 2,6-dione | |
| 10 | Int-25 & 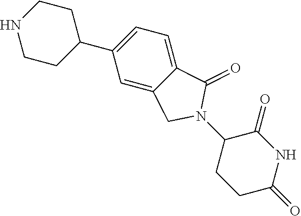 [SMILES: O=C1CCC(N2Cc3cc(C4CCNCC4)ccc3C2=O)C(=O)N1] | 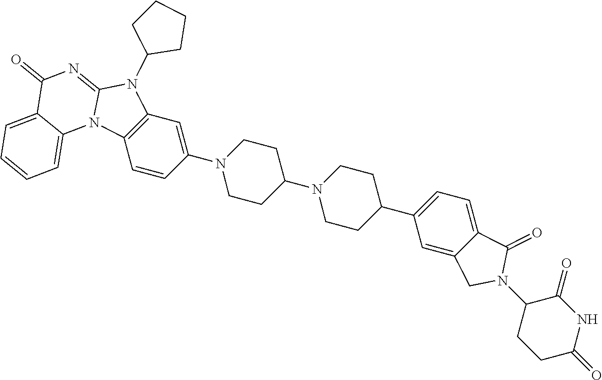 [SMILES: O=C1CCC(N2Cc3cc(C4CCN(C5CCN(c6ccc7c(c6)n(C6CCCC6)c6nc(=O)c8ccccc8n76)CC5)CC4)ccc3C2=O)C(=O)N1] | ¹H NMR (400 MHz, DMSO- d₆): δ 10.98 (s, 1H), 8.41 (d, J = 8.4 Hz, 1H), 8.25 (dd, J = 8.0, 1.2 Hz, 2H), 7.87 (dt, J = 8.8, 1.6 Hz, 1H), 7.65 (d, J = 8.0 Hz, 1H), 7.54 (t, J = 8.0 Hz, 1H), 7.50 (s, 1H), 7.41 (d, J = 8.0 Hz, 1H), 7.16 (d, J = 2.0 Hz, 1H), 7.01 (dd, J = 9.2, 2.0 Hz, 1H), 5.28 (t, J = 8.0 Hz, 1H), 5.09 (dd, J = 13.2, 4.8 Hz, 1H), 4.43 (d, J = 17.6 Hz, 1H), 4.29 (d, J = 17.6 Hz, 1H), 3.88 (d, J = 11.6 Hz, 2H), 3.05 (bs, 2H), 2.98- 2.74 (m, 3H), 2.69-2.55 (m, 2H), 2.47-2.23 (m, 5H), 2.09- 1.87 (m, 8H), 1.87-1.59 (m, 8H); LC-MS: m/z 712.5 (M − H). |
| | | 3-(5-(1′-(7-cyclopenty1-5-oxo- | |
| | | 5,7- | |
| | | dihydrobenzo[4,5]imidazo[1,2- | |
| | | a]quinazolin-9-yl)-[1,4′- | |
| | | bipiperidin]-4-yl)-1- | |
| | | oxoisoindolin-2-yl)piperidine- | |
| | | 2,6-dione | |
| 11 | Int-10 & Int-3 | 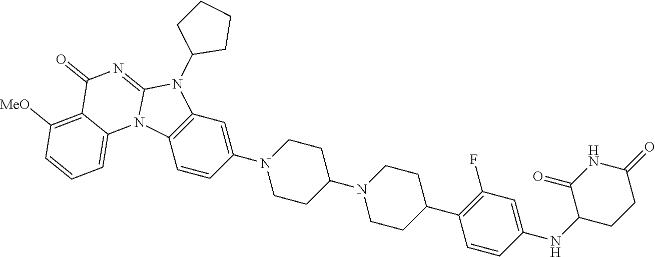 [SMILES: COc1cccc2c1c(=O)nc1n(C3CCCC3)c3cc(N4CCC(N5CCC(c6ccc(NC7CCC(=O)NC7=O)cc6F)CC5)CC4)ccc3n21] | ¹H NMR (400 MHz, DMSO- d₆): δ 10.79 (s, 1H), 8.31 (d, J = 8.8 Hz, 1H), 7.60 (t, J = 8.0 Hz, 1H), 7.10 (m, 2H), 7.06-6.93 (m, 2H), 6.80 (d, J = 8.4 Hz, 1H), 6.47-6.43 (m, 2H), 6.00 (d, J = 6.8 Hz, 1H), 5.25 (t, J = 8.8 Hz, 1H), 4.34-4.28 (m, 1H), 3.91 (s, 3H), 3.88 (bs, 2H), 3.04-2.95 (m, 1H), 2.83-2.56 (m, 5H), 2.38-2.25 (m, 3H), 2.13-1.87 (m, 9H), 1.82-1.43 (m, 9H); LC-MS: m/z 720.5 (M − H). |
| | | 3-((4-(1′-(7-cyclopentyl-4- | |
| | | methoxy-5-oxo-5,7- | |
| | | dihydrobenzo[4,5]imidazo[1,2- | |
| | | a]quinazolin-9-yl)-[1,4′- | |
| | | bipiperidin]-4-yl)-3- | |
| | | fluorophenyl)amino)piperidine- | |
| | | 2,6-dione | |
| 12 | Int-17 & Int-3 | 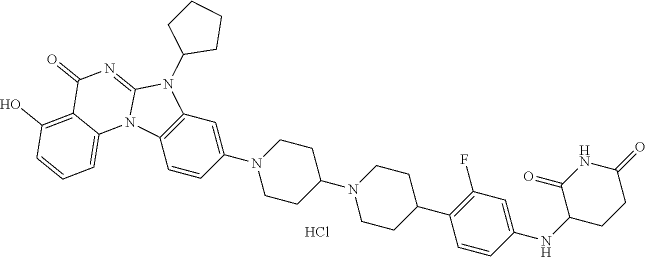 [SMILES: O=C1CCC(Nc2ccc(C3CCN(C4CCN(c5ccc6c(c5)n(C5CCCC5)c5nc(=O)c7c(O)cccc7n65)CC4)CC3)c(F)c2)C(=O)N1 ; Cl] | ¹H NMR (400 MHz, DMSO- d₆): δ 11.67 (s, 1H), 10.80 (s, 1H), 8.23 (d, J = 9.2 Hz, 1H), 7.60 (t, J = 8.4 Hz, 1H), 7.10 (d, J = 1.6 Hz, 1H). 7.04-6.98 (m, 3H), 6.60 (d, J = 8.0 Hz, 1H), 6.46-6.45 (m, 2H), 5.99 (d, J = 7.6 Hz, 1H), 5.27 (t, J = 8.4 Hz, 1H), 4.32-4.29 (m, 1H), 3.88 (d, J = 12 Hz, 2H), 3.00 ( d, J = 12 Hz, 2H), 2.82-2.62 (m, 4H), 2.39-2.22 (m, 4H), 2.12-1.96 (m, 6H), 1.95-1.72 (m, 6H), 1.71-1.55 (m, 6H); LC-MS: m/z 706.5 (M + H). |
| | | 3-((4-(1′-(7-cyclopentyl-4- | |
| | | hydroxy-5-oxo-5,7- | |
| | | dihydrobenzo[4,5]imidazo[1,2- | |
| | | a]quinazolin-9-yl)-[1,4′- | |
| | | bipiperidin]-4-yl)-3- | |
| | | fluorophenyl)amino)piperidine- | |
| | | 2,6-dione-HCl salt | |
| 13 | Int-12 & 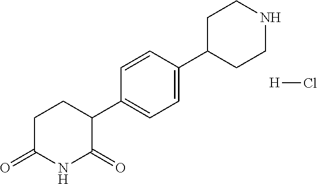 | 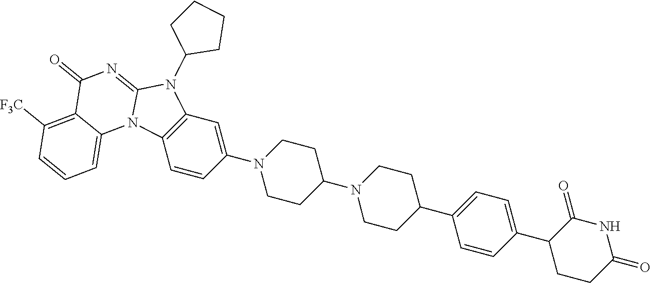 [SMILES: O=C1CCC(c2ccc(C3CCN(C4CCN(c5ccc6c(c5)n(C5CCCC5)c5nc(=O)c7c(C(F)(F)F)cccc7n65)CC4)CC3)cc2)C(=O)N1] | ¹H NMR (400 MHz, DMSO- d₆): δ 10.85 (s, 1H),9.11 (bs, 1H), 8.39 (d, J = 9.2 Hz, 1H), 7.89-7.74 (m, 2H), 7.76-7.73 (m, 1H), 7.25-7.15 (m, 4H), 7.00 (dd, J = 8.8, 1.2 Hz, 1H), 5.31 (t, J = 8.4 Hz, 1H), 4.06 (d, J = 12.8 Hz, 2H), 3.85-3.83 (m, 1H), 3.63 (d, J = 11.6 Hz, 2H), 3.52-3.48 (m, 1H), 3.25-3.14 (m, 2H), 2.93-2.86 (m, 3H), 2.72-2.59 (m, 2H), 2.46-2.32 (m, 2H), 2.28-20 (m, 10H), 1.98-1.73 (m, 6H); LC-MS: m/z 725.5 (M − H). |
| | | 3-(4-(1′-(7-cyclopentyl-5-oxo-4- | |
| | | (trifluoromethyl)-5,7- | |
| | | dihydrobenzo[4,5]imidazo[1,2- | |
| | | a]quinazolin-9-yl)-[1,4′- | |
| | | bipiperidin]-4- | |
| | | yl)phenyl)piperidine-2,6-dione | |
| 14 | Int-21 & 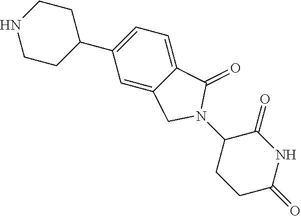 [SMILES: O=C1CCC(N2Cc3cc(C4CCNCC4)ccc3C2=O)C(=O)N1] | 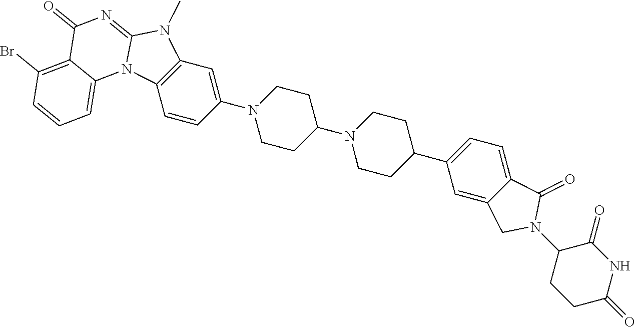 [SMILES: Cn1c2cc(N3CCC(N4CCC(c5ccc6c(c5)CN(C5CCC(=O)NC5=O)C6=O)CC4)CC3)ccc2n2c3cccc(Br)c3c(=O)nc12] | LC-MS: m/z 736.4 (M + H); HPLC purity: 92.6%. |
| | | 3-(5-(1′-(4-bromo-7-methyl-5- | |
| | | oxo-5,7- | |
| | | dihydrobenzo[4,5]imidazo[1,2- | |
| | | a]quinazolin-9-yl)-[1,4′- | |
| | | bipiperidin]-4-yl)-1- | |
| | | oxoisoindolin-2-yl)piperidine- | |
| | | 2,6-dione | |
| 15 | Int-7 & 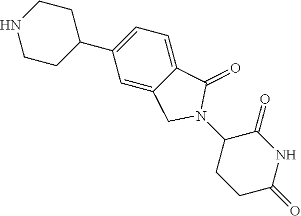 [SMILES: O=C1CCC(N2Cc3cc(C4CCNCC4)ccc3C2=O)C(=O)N1] | 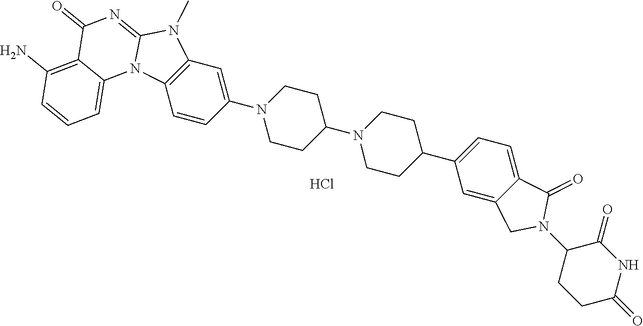 [SMILES: Cn1c2cc(N3CCC(N4CCC(c5ccc6c(c5)CN(C5CCC(=O)NC5=O)C6=O)CC4)CC3)ccc2n2c3cccc(N)c3c(=O)nc12 ; Cl] | ¹H NMR (400 MHz, DMSO- d₆): δ 10.99 (s, 1H), 10.01 (bs, 1H), 8.12 (d, J = 9.2 Hz, 1H), 7.73 (d, J = 8.4 Hz, 1H), 7.48- 7.40 (m, 3H), 7.31 (d, J = 8.0 Hz, 1H), 7.26 (d, J = 2.0 Hz, 1H), 7.02 (dd, J = 9.6, 2.0 Hz, 1H), 6.68 ( d, J = 8.0 Hz, 1H), 5.12 (dd, J = 13.2, 4.8 Hz, 1H), 4.46 (d, J = 17.6 Hz, 1H), 4.33 (d, J = 17.6 Hz, 1H), 4.06 (d, J = 13.2 Hz, 2H), 3.67 (s, 3H), 3.72-3.48 (m, 4H), 3.23-3.12 (m, 2H), 3.10-2.82 (m, 4H), 2.61-41 (m, 2H), 2.29-2.22 (m, 2H), 2.15-1.82 (m, 7H); LC- MS: m/z 673.3 (M − H). |
| | | 3-(5-(1′-(4-amino-7-methyl-5- | |
| | | oxo-5,7- | |
| | | dihydrobenzo[4,5]imidazo[1,2- | |
| | | a]quinazolin-9-yl)-[1,4′- | |
| | | bipiperidin]-4-yl)-1- | |
| | | oxoisoindolin-2-yl)piperidine- | |
| | | 2,6-dione-HCl salt | |
| 16 | Int-21 & Int-3 | 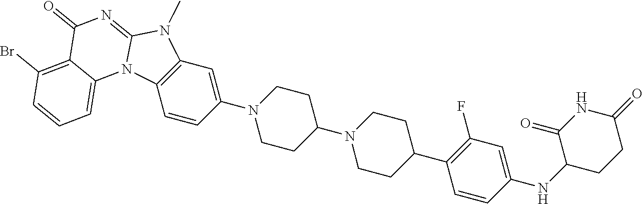 [SMILES: Cn1c2cc(N3CCC(N4CCC(c5ccc(NC6CCC(=O)NC6=O)cc5F)CC4)CC3)ccc2n2c3cccc(Br)c3c(=O)nc12] | LC-MS: m/z 714.4 (M + H); HPLC purity: 93.5%. |
| | | 3-((4-(1′-(4-bromo-7-methyl-5- | |
| | | oxo-5,7- | |
| | | dihydrobenzo[4,5]imidazo[1,2- | |
| | | a]quinazolin-9-yl)-[1,4′- | |
| | | bipiperidin]-4-yl)-3- | |
| | | fluorophenyl)amino)piperidine- | |
| | | 2,6-dione | |
| 17 | Int-21 & 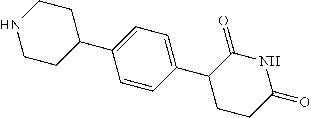 [SMILES: O=C1CCC(c2ccc(C3CCNCC3)cc2)C(=O)N1] | 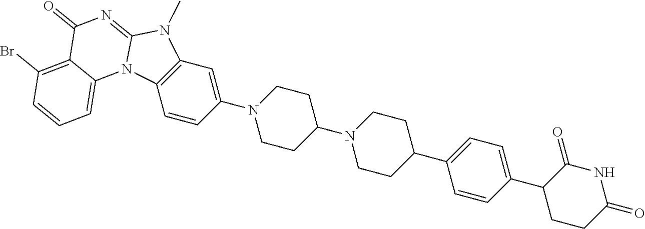 [SMILES: Cn1c2cc(N3CCC(N4CCC(c5ccc(C6CCC(=O)NC6=O)cc5)CC4)CC3)ccc2n2c3cccc(Br)c3c(=O)nc12] | LC-MS: m/z 681.3 (M + H); HPLC purity: 87.1%. |
| | | 3-(4-(1′-(4-bromo-7-methyl-5- | |
| | | oxo-5,7- | |
| | | dihydrobenzo[4,5]imidazo[1,2- | |
| | | a]quinazolin-9-yl)-[1,4′- | |
| | | bipiperidin]-4- | |
| | | yl)phenyl)piperidine-2,6-dione | |
| 18 | Int-19 & 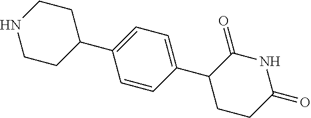 [SMILES: O=C1CCC(c2ccc(C3CCNCC3)cc2)C(=O)N1] | 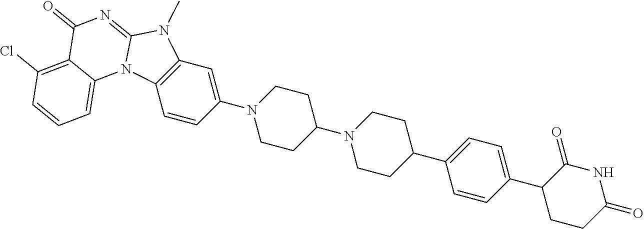 [SMILES: Cn1c2cc(N3CCC(N4CCC(c5ccc(C6CCC(=O)NC6=O)cc5)CC4)CC3)ccc2n2c3cccc(Cl)c3c(=O)nc12] | ¹H NMR (400 MHz, DMSO- d₆): δ 10.82 (s, 1H), 8.35 (d, J = 8.0 Hz, 1H), 8.15 (d, J = 8.8 Hz, 1H), 7.77 (t, J = 8.0 Hz, 1H), 7.54 (d, J = 7.2 Hz, 1H), 7.22- 7.13 (m, 5H), 6.96 (dd, J = 8.8, 1.2 Hz, 1H), 3.92 (d, J = 12.4 Hz, 2H), 3.83-3.79 (m, 1H), 3.64 (s, 3H), 3.03 (d, J = 9.6 Hz, 2H), 2.78 (t, J = 12 Hz, 2H), 2.71-2.59 (m, 2H), 2.48-2.42 (m, 2H), 2.34-2.12 (m, 3H), 2.06-1.87 (m, 3H), 1.82-1.73 (m, 2H), 1.69-1.57 (m, 4H); |
| | | 3-(4-(1′-(4-chloro-7-methyl-5- | LC-MS: m/z 637.4 (M + H). |
| | | oxo-5,7- | |
| | | dihydrobenzo[4,5]imidazo[1,2- | |
| | | a]quinazolin-9-yl)-[1,4′- | |
| | | bipiperidin]-4- | |
| | | yl)phenyl)piperidine-2,6-dione | |
| 19 | Int-13 & 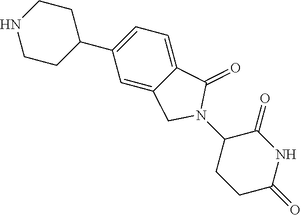 [SMILES: O=C1CCC(N2Cc3cc(C4CCNCC4)ccc3C2=O)C(=O)N1] | 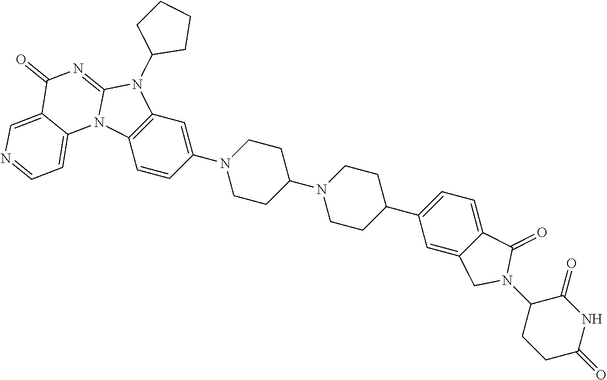 [SMILES: O=C1CCC(N2Cc3cc(C4CCN(C5CCN(c6ccc7c(c6)n(C6CCCC6)c6nc(=O)c8cnccc8n76)CC5)CC4)ccc3C2=O)C(=O)N1] | ¹H NMR (400 MHz, DMSO- d₆): δ 10.98 (s, 1H), 9.31 (bs, 1H), 8.63 (d, J = 6.0 Hz, 1H), 8.30 (d, J = 8.8 Hz, 1H), 7.65 (d, J = 8.0 Hz, 1H), 7.50 (s, 1H), 7.44-7.39 (m, 2H), 7.14 (s, 1H), 7.04 (d, J = 9.2 Hz, 1H), 5.35- 5.31 (m, 1H), 5.10 (dd, J = 13.2, 4.8 Hz, 1H), 4.43 (d, J = 17.2 Hz, 1H), 4.29 (d, J = 17.2 Hz, 1H), 3.90 (d, J = 12.0 Hz, 2H), 3.11-3.03 (m, 2H), 2.99-2.75 (m, 3H), 2.72-2.59 (m, 2H), 2.47-2.25 (m, 5H), 2.14-1.90 (m, 8H), 1.89-1.60 (m, 8H); LC-MS: m/z 713.5 (M − H). |
| | | 3-(5-(1′-(7-cyclopentyl-5-oxo- | |
| | | 5,7- | |
| | | dihydrobenzo[4,5]imidazo[1,2- | |
| | | a]pyrido[3,4-e]pyrimidin-9-yl)- | |
| | | [1,4′-bipiperidin]-4-yl)-1- | |
| | | oxoisoindolin-2-yl)piperidine- | |
| | | 2,6-dione | |
| 20 | Int-7 & 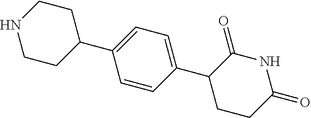 [SMILES: O=C1CCC(c2ccc(C3CCNCC3)cc2)C(=O)N1] | 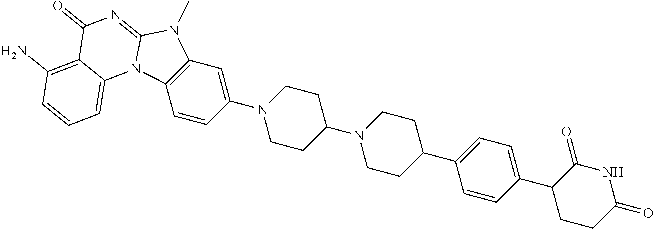 [SMILES: Cn1c2cc(N3CCC(N4CCC(c5ccc(C6CCC(=O)NC6=O)cc5)CC4)CC3)ccc2n2c3cccc(N)c3c(=O)nc12] | ¹H NMR (400 MHz, DMSO- d₆): δ 10.83 (s, 1H), 8.05 (d, J = 9.2 Hz, 1H), 7.41 (t, J = 8.0 Hz, 1H), 7.27 (d, J = 8.4 Hz, 1H), 7.21 (d, J = 8.0 Hz, 2H), 7.18- 7.12 (m, 3H), 6.94 (dd, J = 9.2, 2.0 Hz, 1H), 6.64 (d, J = 8.0 Hz, 1H), 3.86 (d, J = 18.0 Hz, 2H), 3.82-3.79 (m, 1H), 3.63 (s, 3H), 3.11-2.98 (m, 2H), 2.78 (t, J = 11.2 Hz, 2H), 2.70-2.59 (m, 2H), 2.48-2.44 (m, 2H), 2.35- 2.12 (m, 3H), 2.08-1.88 (m, 3H), 1.87-1.71 (m, 2H), 1.72- |
| | | 3-(4-(1′-(4-amino-7-methyl-5- | 1.49 (m, 4H); LC-MS: m/z |
| | | oxo-5,7- | 618.4.5 (M + H). |
| | | dihydrobenzo[4,5]imidazo[1,2- | |
| | | a]quinazolin-9-yl)-[1,4′- | |
| | | bipiperidin]-4- | |
| | | yl)phenyl)piperidine-2,6-dione | |
| 21 | Int-6 & 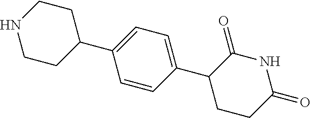 [SMILES: O=C1CCC(c2ccc(C3CCNCC3)cc2)C(=O)N1] | 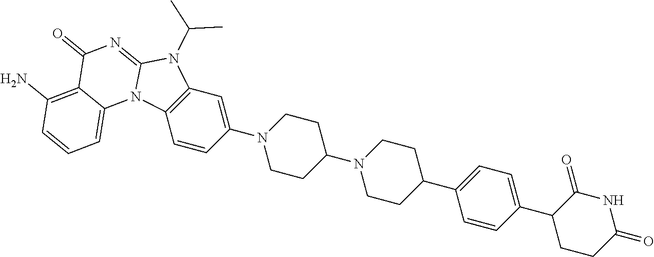 [SMILES: CC(C)n1c2cc(N3CCC(N4CCC(c5ccc(C6CCC(=O)NC6=O)cc5)CC4)CC3)ccc2n2c3cccc(N)c3c(=O)nc12] | LC-MS: m/z 646.4 (M + H); HPLC purity: 99.5%. |
| | | 3-(4-(1′-(4-amino-7-isopropyl-5- | |
| | | oxo-5,7- | |
| | | dihydrobenzo[4,5]imidazo[1,2- | |
| | | a]quinazolin-9-yl)-[1,4′- | |
| | | bipiperidin]-4- | |
| | | yl)phenyl)piperidine-2,6-dione | |
| 22 | Int-18 & 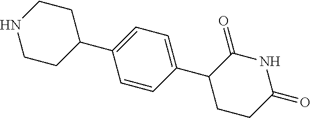 [SMILES: O=C1CCC(c2ccc(C3CCNCC3)cc2)C(=O)N1] | 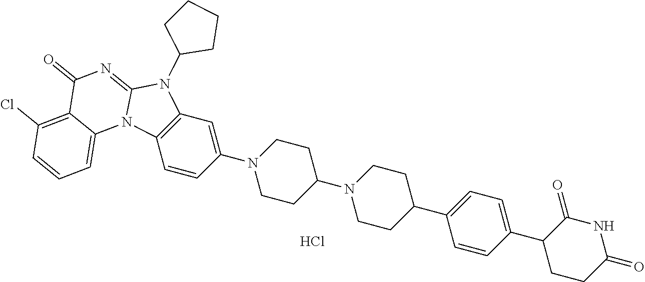 [SMILES: O=C1CCC(c2ccc(C3CCN(C4CCN(c5ccc6c(c5)n(C5CCCC5)c5nc(=O)c7c(Cl)cccc7n65)CC4)CC3)cc2)C(=O)N1 ; Cl] | ¹H NMR (400 MHz, DMSO- d₆): δ 10.84 (s, 1H), 10.72 (bs, 1H), 8.38 (d, J = 8.4 Hz, 1H), 8.24 ( d, J = 9.2 Hz, 1H) 7.78 (t, J = 8.0 Hz, 1H), 7.56 (d, J = 7.2 Hz, 1H), 7.26-7.18 (m, 5H), 7.00 (d, J = 8.8 Hz, 1H), 5.25 (t, J = 8.8 Hz, 1H), 4.05-3.95 (m, 2H), 3.84 (dd, J = 11.6, 4.8 Hz, 1H), 3.64--3.57 (m, 2H), 3.49- 3.41 (m, 1H), 3.20-3.17 (m, 2H), 2.93-2.84 (m,3H), 2.75- 2.62 (m, 1H), 2.48-2.45 (1H), 2.39-2.11 (m, 7H), 2.09-1.88 (m, 9H), 1.81-1.67 (m, 2H); LC- MS: m/z 691.5 (M + H). |
| | | 3-(4-(1′-(4-chloro-7-cyclopentyl- | |
| | | 5-oxo-5,7- | |
| | | dihydrobenzo[4,5]imidazo[1,2- | |
| | | a]quinazolin-9-yl)-[1,4′- | |
| | | bipiperidin]-4- | |
| | | yl)phenyl)piperidine-2,6-dione- | |
| | | HCl salt | |
| 23 | Int-18 & Int-3 | 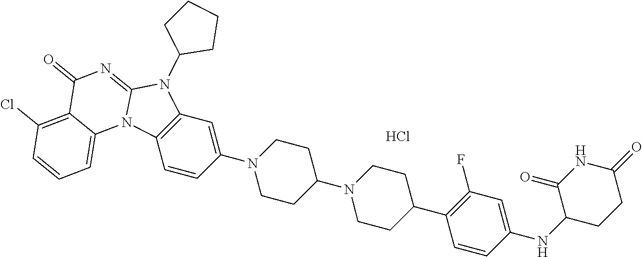 [SMILES: O=C1CCC(Nc2ccc(C3CCN(C4CCN(c5ccc6c(c5)n(C5CCCC5)c5nc(=O)c7c(Cl)cccc7n65)CC4)CC3)c(F)c2)C(=O)N1 ; Cl] | ¹H NMR (400 MHz, DMSO- d₆): δ 10.80 (s, 1H), 10.67 (bs, 1H), 8.40 (d, J = 8.4 Hz, 1H), 8.28 (d, J = 8.8 Hz, 1H), 7.79 (t, J = 8.4 Hz, 1H), 7.57 (d, J = 7.6 Hz, 1H), 7.33 (bs, 1H), 7.15 (d, J = 8.4 Hz, 1H), 6.97 (t, J = 8.8 Hz, 1H), 6.53-6.46 (m, 2H), 5.24 (t, J = 8.4 Hz, 1H), 4.35- 4.31 (m, 1H), 3.99 (d, J = 12 Hz, 2H), 3.60-3.51 (m, 2H), 3.48- 3.38 (m, 1H), 3.22-3.08 (m, 2H), 3.07-2.90 (m, 3H), 2.79- 2.53 (m, 2H), 2.36-2.25 (m, 4H), 2.21-1.83 (m, 12H), 1.81- 1.68 (m, 2H); LC-MS: m/z |
| | | 3-((4-(1′-(4-chloro-7- | 724.4 (M − H). |
| | | cyclopentyl-5-oxo-5,7- | |
| | | dihydrobenzo[4,5]imidazo[1,2- | |
| | | a]quinazolin-9-yl)-[1,4′- | |
| | | bipiperidin]-4-yl)-3- | |
| | | fluorophenyl)amino)piperidine- | |
| | | 2,6-dione-HCl salt | |
| 24 | Int-14 & 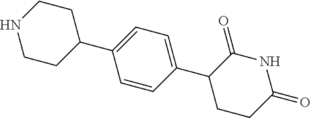 [SMILES: O=C1CCC(c2ccc(C3CCNCC3)cc2)C(=O)N1] | 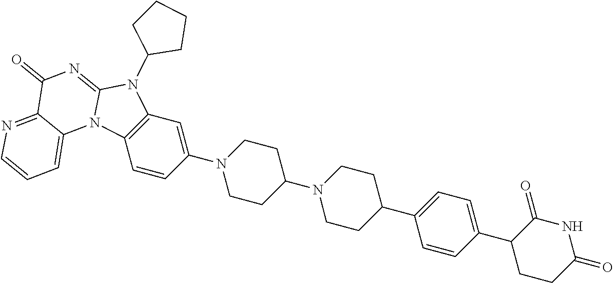 [SMILES: O=C1CCC(c2ccc(C3CCN(C4CCN(c5ccc6c(c5)n(C5CCCC5)c5nc(=O)c7ncccc7n65)CC4)CC3)cc2)C(=O)N1] | LC-MS: m/z 658.4 (M + H). HPLC purity: 93.2%. |
| | | 3-(4-(1′-(7-cyclopentyl-5-oxo- | |
| | | 5,7- | |
| | | dihydrobenzo[4,5]imidazo[1,2- | |
| | | a]pyrido[2,3-e]pyrimidin-9-yl)- | |
| | | [1,4′-bipiperidin]-4- | |
| | | yl)phenyl)piperidine-2,6-dione | |
| 25 | Int-5 & 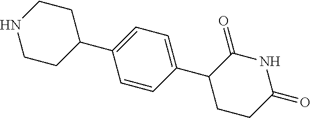 [SMILES: O=C1CCC(c2ccc(C3CCNCC3)cc2)C(=O)N1] | 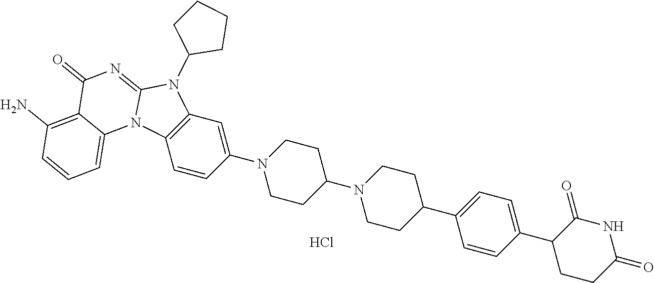 [SMILES: Nc1cccc2c1c(=O)nc1n(C3CCCC3)c3cc(N4CCC(N5CCC(c6ccc(C7CCC(=O)NC7=O)cc6)CC5)CC4)ccc3n21 ; Cl] | ¹H NMR (400 MHz, DMSO- d₆): δ 11.05 (bs, 1H), 10.83 (s, 1H), 8.19 (d, J = 8.8 Hz, 1H), 7.44 (t, J = 8.0 Hz, 1H), 7.35 (d, J = 8.0 Hz, 1H), 7.27 (bs, 1H), 7.25-7.17 (m, 5H), 7.12 (d, J = 7.2 Hz, 1H), 7.00 (d, J = 8.0, 1.6 Hz, 1H), 5.25 (t, J = 8.4 Hz, 1H), 3.87 (d, J = 11.6 Hz, 2H), 3.85 (dd, J = 11.6, 5.2 Hz, 1H), 3.63-3.54 (m, 2H), 3.51-3.42 (m, 1H) 3.20-3.07 (m, 2H), 3.04- 2.83 (m, 3H), 2.73-2.64 (m, 1H), 2.53-2.48 (1H), 2.38- 2.13 (m, 7H), 2.12-1.94 (m, 9H), 1.82-1.69 (m, 2H); LC- MS: m/z 672.5 (M + H). |
| | | 3-(4-(1′-(4-amino-7-cyclopentyl- | |
| | | 5-oxo-5,7- | |
| | | dihydrobenzo[4,5]imidazo[1,2- | |
| | | a]quinazolin-9-yl)-[1,4′- | |
| | | bipiperidin]-4- | |
| | | yl)phenyl)piperidine-2,6-dione- | |
| | | HCl salt | |
| 26 | Int-5 & Int-1 | 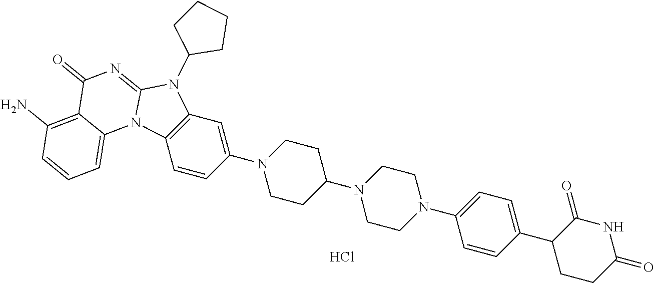 [SMILES: Nc1cccc2c1c(=O)nc1n(C3CCCC3)c3cc(N4CCC(N5CCN(c6ccc(C7CCC(=O)NC7=O)cc6)CC5)CC4)ccc3n21 ; Cl] | ¹H NMR (400 MHz, DMSO- d₆): δ 10.98 (bs, 1H), 10.80 (s, 1H), 8.21 (d, J = 9.1 Hz, 1H), 7.46 (t, J = 8.0 Hz, 1H), 7.37 (d, J = 8.4 Hz, 1H), 7.26 (bs, 1H), 7.27-7.11 (m, 3H), 6.99 (d, J = 8.0 Hz, 2H), 6.71 (d, J = 8.0, 1.6 Hz, 1H), 5.27 (t, J = 8.8 Hz, 1H), 4.00 (d, J = 12.0 Hz, 1H), 3.84 (d, J = 9.2 Hz, 2H), 3.78 (dd, J = 11.2, 4.8 Hz, 1H), 3.69-- 3.62 (m, 2H), 3.53-3.45 (m, 1H) 3.25-3.16 (m, 4H), 2.97- 2.88 (m,2H), 2.71-2.61 (m, 1H), 2.47-2.44 (1H), 2.38-2.23 (m, 5H), 2.22-2.11 (m, 1H), 2.09-1.92 (m, 8H), 1.81-1.70 (m, 2H); LC-MS: m/z 673.4 |
| | | 3-(4-(4-(1-(4-amino-7- | (M + H). |
| | | cyclopentyl-5-oxo-5,7- | |
| | | dihydrobenzo[4,5]imidazo[1,2- | |
| | | a]quinazolin-9-yl)piperidin-4- | |
| | | yl)piperazin-1- | |
| | | yl)phenyl)piperidine-2,6-dione- | |
| | | HCl salt | |
| 27 | Int-19 & 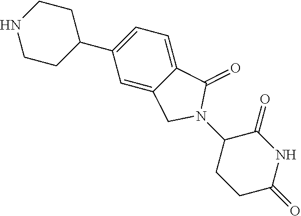 [SMILES: O=C1CCC(N2Cc3cc(C4CCNCC4)ccc3C2=O)C(=O)N1] | 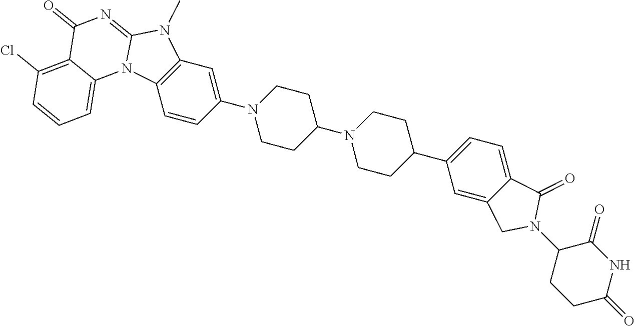 [SMILES: Cn1c2cc(N3CCC(N4CCC(c5ccc6c(c5)CN(C5CCC(=O)NC5=O)C6=O)CC4)CC3)ccc2n2c3cccc(Cl)c3c(=O)nc12] | ¹H NMR (400 MHz, DMSO- d₆): δ 10.98 (s, 1H), 8.35 (d, J = 8.4 Hz, 1H), 8.15 (d, J = 9.2 Hz, 1H), 7.77 (t, J = 8.4 Hz, 1H), 7.65 (d, J = 8.0 Hz, 1H), 7.55 (d, J = 7.2 Hz, 1H), 7.50 (s, 1H), 7.41 (d, J = 8.4 Hz, 1H), 7.19 (d, J = 2.0 Hz, 1H), 6.97 (dd, J = 8.8, 2.0 Hz, 1H), 5.10 (dd, J = 13.2, 5.2 Hz, 1H), 4.43 (d, J = 17.6 Hz, 1H), 4.29 (d, J = 17.6 Hz, 1H), 3.92 (d, J = 12.0 Hz, 2H), 3.64 (s, 3H), 3.07-3.01 (m, 2H), 2.98-2.87 (m, 1H), 2.86- 2.76 (m, 2H), 2.65-2.56 (m, 2H), 2.42-2.25 (m, 3H), 2.04- 1.96 (m, 2H), 1.95-1.78 (m, 4H), 1.77-1.58 (m, 4H); LC- MS: m/z 692.4 (M + H). |
| | | 3-(5-(1′-(4-chloro-7-methyl-5- | |
| | | oxo-5,7- | |
| | | dihydrobenzo[4,5]imidazo[1,2- | |
| | | a]quinazolin-9-yl)-[1,4′- | |
| | | bipiperidin]-4-yl)-1- | |
| | | oxoisoindolin-2-yl)piperidine- | |
| | | 2,6-dione | |
| 28 | Int-20 & 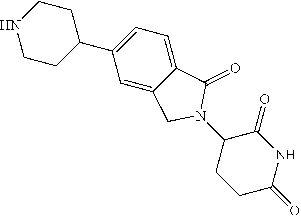 [SMILES: O=C1CCC(N2Cc3cc(C4CCNCC4)ccc3C2=O)C(=O)N1] | 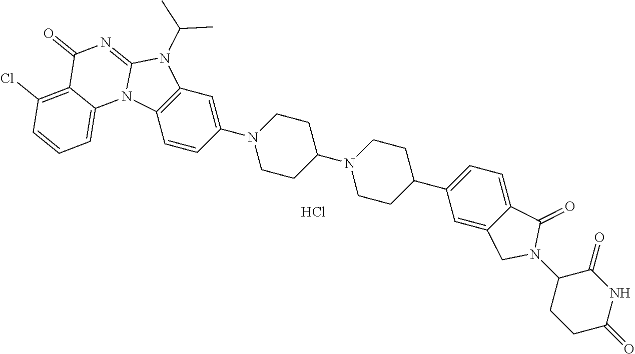 [SMILES: CC(C)n1c2cc(N3CCC(N4CCC(c5ccc6c(c5)CN(C5CCC(=O)NC5=O)C6=O)CC4)CC3)ccc2n2c3cccc(Cl)c3c(=O)nc12 ; Cl] | ¹H NMR (400 MHz, DMSO- d₆): δ 10.99 (s, 1H), 10.88 (bs, 1H), 8.39 (d, J = 8.8 Hz, 1H), 8.26 (d, J = 9.2 Hz, 1H), 7.78 (t, J = 8.0 Hz, 1H), 7.72 (d, J = 8.0 Hz, 1H), 7.57 (d, J = 7.6 Hz, 1H), 7.43 (s, 1H), 7.43-7.36 (m, 2H), 7.10 (d, J = 8.0 Hz, 1H), 5.16-5.11 (m, 1H), 4.47 (d, J = 17.2 Hz, 1H), 4.33 (d, J = 17.6 Hz, 1H), 4.10-3.90 (m, 3H), 3.65-3.58 (m, 2H), 3.51-3.42 (m, 1H), 3.23-3.12 (m, 2H), 3.10-3.01 (m, 1H), 3.00-2.87 (m, 2H), 2.66-2.57 (m, 1H), 2.47-2.38 (m, 1H), 2.37-2.18 (m, 5H), 2.11-1.92 (m, 5H), 1.63 (d, J= 7.2 Hz, 6H); LC- MS: m/z 720.5 (M + H). |
| | | 3-(5-(1′-(4-chloro-7-isopropyl-5- | |
| | | oxo-5,7- | |
| | | dihydrobenzo[4,5]imidazo[1,2- | |
| | | a]quinazolin-9-yl)-[1,4′- | |
| | | bipiperidin]-4-yl)-1- | |
| | | oxoisoindolin-2-yl)piperidine- | |
| | | 2,6-dione-HCl salt | |
| 29 | Int-20 & Int-3 | 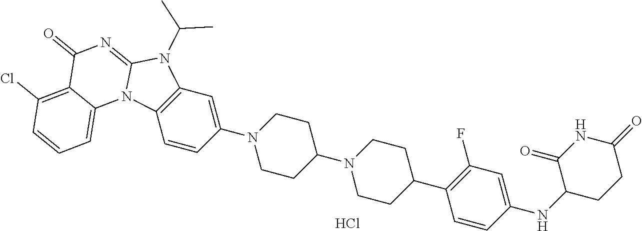 [SMILES: CC(C)n1c2cc(N3CCC(N4CCC(c5ccc(NC6CCC(=O)NC6=O)cc5F)CC4)CC3)ccc2n2c3cccc(Cl)c3c(=O)nc12 ; Cl] | ¹H NMR (400 MHz, DMSO- d₆): δ 10.80 (s, 1H), 10.46 (bs, 1H), 8.39 (d, J = 8.4 Hz, 1H), 8.25 (d, J = 8.8 Hz, 1H), 7.78 (t, J = 8.4 Hz, 1H), 7.57 (d, J = 8.0 Hz, 1H), 7.36 (bs, 1H), 7.08 (d, J = 8.8 Hz, 1H), 6.97 (t, J = 8.4 Hz, 1H), 6.52-6.46 (m, 2H), 5.14 (t, J = 7.2 Hz, 1H), 4.33 (dd, J = 11.2, 4.8 Hz, 1H), 4.03 (d, J = 10.8 Hz, 2H), 3.57-3.38 (m, 4H), 3.23-3.09 (m, 2H), 3.07-2.86 (m, 3H), 2.81-2.56 (m, 2H), 2.32-2.23 (m, 2H), |
| | | 3-((4-(1′-(4-chloro-7-isopropyl- | 2.20-2.03 (m, 3H), 2.01-1.84 |
| | | 5-oxo-5,7- | (m, 5H), 1.62 (d, J = 6.8 Hz, |
| | | dihydrobenzo[4,5]imidazo[1,2- | 6H); LC-MS: m/z 698.4 |
| | | a]quinazolin-9-yl)-[1,4′- | (M + H). |
| | | bipiperidin]-4-yl)-3- | |
| | | fluorophenyl)amino)piperidine- | |
| | | 2,6-dione-HCl salt | |
| 30 | Int-18 & 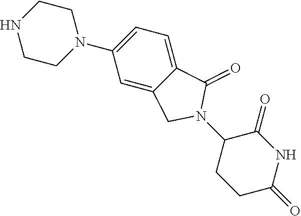 [SMILES: O=C1CCC(N2Cc3cc(N4CCNCC4)ccc3C2=O)C(=O)N1] | 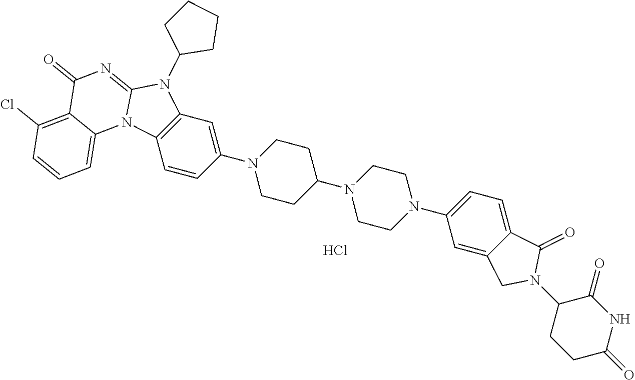 [SMILES: O=C1CCC(N2Cc3cc(N4CCN(C5CCN(c6ccc7c(c6)n(C6CCCC6)c6nc(=O)c8c(Cl)cccc8n76)CC5)CC4)ccc3C2=O)C(=O)N1 ; Cl] | ¹H NMR (400 MHz, DMSO- d₆): δ 11.02 (bs, 1H), 10.96 (s, 1H), 8.39 (d, J = 8.4 Hz, 1H), 8.24 (d, J = 9.2 Hz, 1H), 7.78 (t, J = 8.4 Hz, 1H), 7.61 (d, J = 8.8 Hz, 1H), 7.57 (d, J = 7.6 Hz, 1H), 7.23 (bs, 1H), 7.20-7.16 (m, 2H), 7.08 (m, 1H), 5.23 (t, J = 8.4 Hz, 1H), 5.10 (dd, J = 13.2, 5.2 Hz, 1H), 4.37 (d, J = 16.8 Hz, 1H), 4.25 (d, J = 16.8 Hz, 1H), 4.11-4.00 (m, 5H), 3.70-3.62 (m, 2H), 3.52-3.42 (m, 1H), 3.40-3.19 (m, 4H), 2.95-2.80 (m, 3H), 2.64-2.56 (m, 1H), 2.43-2.26 (m, 5H), 2.09-1.87 (m, 7H), 1.82-1.68 (m, 2H); LC-MS: m/z 747.5 (M + H). |
| | | 3-(5-(4-(1-(4-chloro-7- | |
| | | cyclopentyl-5-oxo-5,7- | |
| | | dihydrobenzo[4,5]imidazo[1,2- | |
| | | a]quinazolin-9-yl)piperidin-4- | |
| | | yl)piperazin-1-yl)-1- | |
| | | oxoisoindolin-2-yl)piperidine- | |
| | | 2,6-dione-HCl salt | |
| 31 | Int-9 & 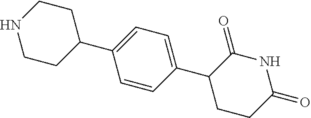 [SMILES: O=C1CCC(c2ccc(C3CCNCC3)cc2)C(=O)N1] | 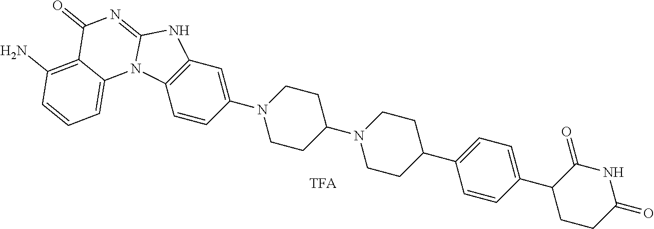 [SMILES: Nc1cccc2c1c(=O)nc1[nH]c3cc(N4CCC(N5CCC(c6ccc(C7CCC(=O)NC7=O)cc6)CC5)CC4)ccc3n12] | ¹H NMR (400 MHz, DMSO- d₆): δ 10.84 (s, 1H), 9.45 (bs, 1H), 8.03 (d, J = 8.8 Hz, 1H), 7.48 (t, J = 8.0 Hz, 1H), 7.31 (d, J = 8.0 Hz, 1H), 7.26-7.19 (m, 4H), 7.16 (s, 1H), 6.99 (d, J = 9.2 Hz, 1H), 6.67 (d, J = 8.4, 1H), 3.94-3.81 (m, 4H), 3.63 (d, J = 11.2 Hz, 2H), 3.47-3.38 (m, 1H), 3.22-3.12 (m, 2H), 2.92-2.76 (m, 3H), 2.73-2.63 (m, 1H), 2.24-2.14 (m, 3H), 2.10-1.82 (m, 7H); LC-MS: m/z 604.4 (M + H). |
| | | 3-(4-(1′-(4-amino-5-oxo-5,7- | |
| | | dihydrobenzo[4,5]imidazo[1,2- | |
| | | a]quinazolin-9-yl)-[1,4′- | |
| | | bipiperidin]-4- | |
| | | yl)phenyl)piperidine-2,6-dione- | |
| | | TFA salt | |
| 32 | Int-7 & Int-1 | 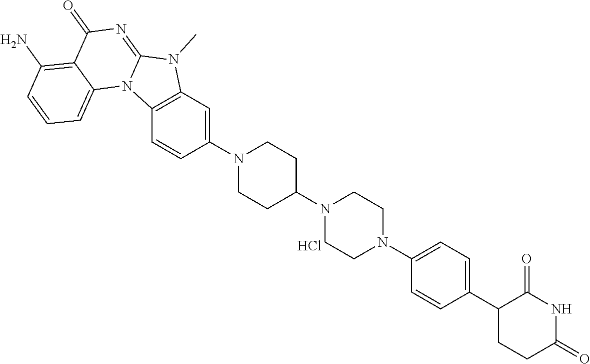 [SMILES: Cn1c2cc(N3CCC(N4CCN(c5ccc(C6CCC(=O)NC6=O)cc5)CC4)CC3)ccc2n2c3cccc(N)c3c(=O)nc12 ; Cl] | ¹H NMR (400 MHz, DMSO- d₆): δ 10.97 (bs, 1H), 10.79 (s, 1H), 8.21 (d, J = 8.8 Hz, 1H), 7.49 (t, J = 8.0 Hz, 1H), 7.42- 7.30 (m, 2H), 7.20-7.08 (m, 3H), 6.99 (d, J = 8.4 Hz, 2H), 6.76 (d, J = 8.4 Hz, 1H), 4.07 (d, J = 11.2 Hz, 2H), 3.87-3.81 (m, 2H), 3.80-3.76 (m, 1H), 3.75 (s, 3H), 3.69-3.62 (m, 2H), 3.48- 3.45 (m, 1H), 3.27-3.15 (m, 4H), 2.90-2.82 (m, 2H), 2.65- 2.56 (m, 2H), 2.36-2.26 (m, 2H), 2.22-2.08 (m, 1H), 2.06- 1.87 (m, 3H); LC-MS: m/z 619.4 (M + H). |
| | | 3-(4-(4-(1-(4-amino-7-methyl-5- | |
| | | oxo-5,7- | |
| | | dihydrobenzo[4,5]imidazo[1,2- | |
| | | a]quinazolin-9-yl)piperidin-4- | |
| | | yl)piperazin-1- | |
| | | yl)phenyl)piperidine-2,6-dione- | |
| | | HCl salt | |
| 33 | Int-11 & Int-3 | 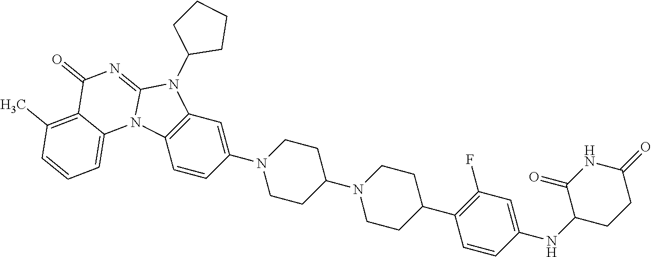 | LC-MS: m/z 700.15 (M + H). HPLC purity: 98.5% . |
| | | 3-((4-(1′-(7-cyclopentyl-4- | |
| | | methyl-5-oxo-5,7- | |
| | | dihydrobenzo[4,5]imidazo[1,2- | |
| | | a]quinazolin-9-yl)-[1,4′- | |
| | | bipiperidin]-4-yl)-3- | |
| | | fluorophenyl)amino)piperidine- | |
| | | 2,6-dione | |
| 34 | Int-22 & Int-3 | 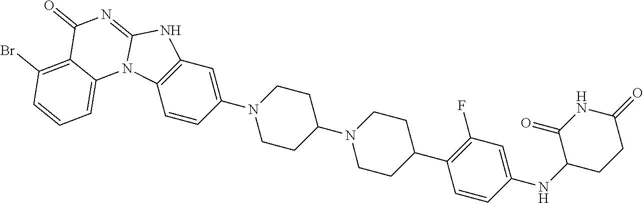 [SMILES: O=C1CCC(Nc2ccc(C3CCN(C4CCN(c5ccc6c(c5)[nH]c5nc(=O)c7c(Br)cccc7n56)CC4)CC3)c(F)c2)C(=O)N1] | LC-MS: m/z 700.15 (M + H). HPLC purity: 89.1% |
| | | 3-((4-(1′-(4-bromo-5-oxo-5,7- | |
| | | dihydrobenzo[4,5]imidazo[1,2- | |
| | | a]quinazolin-9-yl)-[1,4′- | |
| | | bipiperidin]-4-yl)-3- | |
| | | fluorophenyl)amino)piperidine- | |
| | | 2,6-dione | |
| 35 | Int-18 & 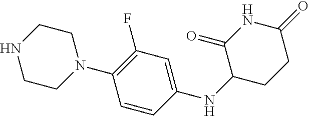 [SMILES: O=C1CCC(Nc2ccc(N3CCNCC3)c(F)c2)C(=O)N1] | 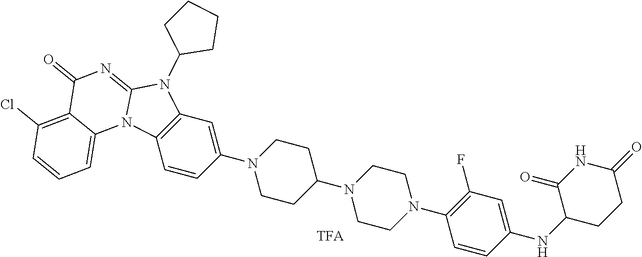 [SMILES: O=C1CCC(Nc2ccc(N3CCN(C4CCN(c5ccc6c(c5)n(C5CCCC5)c5nc(=O)c7c(Cl)cccc7n65)CC4)CC3)c(F)c2)C(=O)N1] | ¹H NMR (400 MHz, DMSO- d₆): δ 10.79 (s, 1H), 9.53 (bs, 1H), 8.37 (d, J = 8.4 Hz, 1H), 8.22 (d, J = 9.2 Hz, 1H), 7.77 (t, J = 8.4 Hz, 1H), 7.56 (d, J = 7.6 Hz, 1H), 7.18 (d, J = 1.6 Hz, 1H), 7.03 (dd, J = 8.8, 2.0 Hz, 1H), 6.91 (t, J = 9.2 Hz, 1H), 6.56 (dd, J = 11.2, 2.4 Hz, 1H), 6.47 (dd, J = 7.2, 2.4 Hz, 1H), 5.23 (t, J = 8.4 Hz, 1H), 4.28 (dd, J = 11.6, 4.8 Hz, 1H), 4.03 (d, J = 12 Hz, 2H), 3.68-3.61 (m, 2H), 3.56-3.48 (m, 1H), 3.38-3.22 (m, 4H), 3.05-2.94 (m, 2H), 2.89-2.80 (m, 2H), |
| | | 3-((4-(4-(1-(4-chloro-7- | 2.79-2.67 (m, 1H), 2.62-2.55 |
| | | cyclopentyl-5-oxo-5,7- | (m, 1H), 2.41-2.20 (m, 4H), |
| | | dihydrobenzo[4,5]imidazo[1,2- | 2.13-1.97 (m, 5H), 1.93-1.71 |
| | | a]quinazolin-9-yl)piperidin-4- | (m, 5H); LC-MS: m/z 725.5 |
| | | yl)piperazin-1-y1)-3- | (M + H). |
| | | fluorophenyl)amino)piperidine- | |
| | | 2,6-dione-TFA salt | |
| 36 | Int-19 & Int-3 | 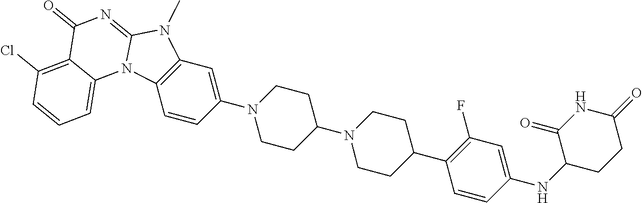 [SMILES: Cn1c2cc(N3CCC(N4CCC(c5ccc(NC6CCC(=O)NC6=O)cc5F)CC4)CC3)ccc2n2c3cccc(Cl)c3c(=O)nc12] | ¹H NMR (400 MHz, DMSO-d₆): δ 10.80 (s, 1H), 9.38 (bs, 1H), 8.36 (d, J = 8.4 Hz, 1H), 8.19 (d, J = 9.2 Hz, 1H), 7.77 (t, J = 8.0 Hz, 1H), 7.56 (d, J = 8.0 Hz, 1H), 7.26 (s, 1H), 7.00-6.99 (m, 2H), 6.51-6.50 (m, 2H), 4.33-4.31 (m, 1H), 4.06 (d, J = 12.0 Hz, 2H), 3.60 (s, 3H), 3.58-3,51 (m, 2H), 3.44-3.42 (m, 1H), 3.21-3.10 (m, 2H), 3.01-2.99 (m, 1H), 2.83-2.79 (m, 2H), 2.76- 2.72 (m, 1H), 2.61-2.59 (m, 1H), 2.21-2.18 (m, 2H), 2.10-2.08 (m, |
| | | 3-((4-(1′-(4-chloro-7-methyl-5- | 1H), 2.00-1.91 (m, 4H), 1.89-1.70 |
| | | oxo-5,7- | (m, 3H); LC-MS: m/z 670.5 |
| | | dihydrobenzo[4,5]imidazo[1,2- | (M + H). |
| | | a]quinazolin-9-yl)-[1,4′- | |
| | | bipiperidin]-4-yl)-3- | |
| | | fluorophenyl)amino)piperidine- | |
| | | 2,6-dione | |
| 37 | Int-8 & 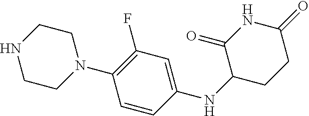 [SMILES: O=C1CCC(Nc2ccc(N3CCNCC3)c(F)c2)C(=O)N1] | 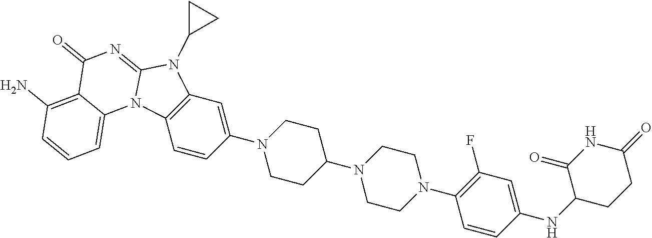 [SMILES: Nc1cccc2c1c(=O)nc1n(C3CC3)c3cc(N4CCC(N5CCN(c6ccc(NC7CCC(=O)NC7=O)cc6F)CC5)CC4)ccc3n21] | ¹H NMR (400 MHz, DMSO-d₆): δ 10.79 (s, 1H), 9.43 (bs, 1H), 8.10 (d, J = 9.2 Hz, 1H), 7.42 (t, J = 8.0 Hz, 1H), 7.28 (d, J = 8.0 Hz, 1H), 7.15 (d, J = 2.4 Hz, 1H), 7.00 (d, J = 6.8 Hz, 1H), 6.91 (t, J = 8.4 Hz, 1H), 6.66 (d, J = 8.0 Hz, 1H), 6.56 (d, J = 14.8 Hz, 1H), 6.46 (d, J = 8.0 Hz, 1H), 4.32-4.28 (m, 1H), 4.00 (d, J = 12.8 Hz, 3H), 3.62- 3.58 (m, 2H), 3.55-3.40 (m, 1H), 3.35-3.30 (m, 2H), 3.18-3.10 (m, 1H), 3.01-2.95 (m, 2H), 2.89-2.81 (m, 2H), 2.60 (s, 1H), 2.25 (d, J = |
| | | 3-((4-(4-(1-(4-amino-7- | 1.6 Hz, 2H), 2.10-2.05 (m, 1H), |
| | | cyclopropyl-5-oxo-5,7- | 1.90-1.71 (m, 3H), 1.24 (d, J = 2.8 |
| | | dihydrobenzo[4,5]imidazo[1,2- | Hz, 4H), 1.09 (t, J = 2.8 Hz, 2H); |
| | | a]quinazolin-9-yl)piperidin-4- | LC-MS: m/z 678.5 (M + H). |
| | | yl)piperazin-1-yl)-3- | |
| | | fluorophenyl)amino)piperidine- | |
| | | 2,6-dione | |
| 38 | Int-18 & Int-3 | 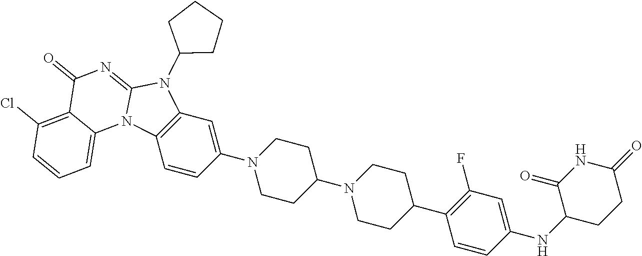 [SMILES: O=C1CCC(Nc2ccc(C3CCN(C4CCN(c5ccc6c(c5)n(C5CCCC5)c5nc(=O)c7c(Cl)cccc7n65)CC4)CC3)c(F)c2)C(=O)N1] | ¹H NMR (400 MHz, DMSO-d₆): δ 10.79 (s, 1H), 8.36 (d, J = 8.3 Hz, 1H), 8.19 (d, J = 9.2 Hz, 1H), 7.77 (t, J = 8.0 Hz, 1H), 7.55 (d, J = 8.0 Hz, 1H), 7.13 (s, 1H), 6.99 (s, 2H), 6.50-6.43 (m, 2H), 5.96 (s, 1H), 5.23-5.19 (m, 1H), 4.33-4.24 (m, 1H), 3.92-3.81 (m, 2H), 3.08-2.91 (m, 2H), 2.83-2.71 (m, 3H), 2.61- 2.59 (m, 2H), 2.48-2.42 (m, 1H), 2.35-2.21 (m, 4H), 2.12-1.95 (m, 6H), 1.91-1.80 (m, 3H), 1.78-1.55 (m, 7H); LC-MS: m/z 724.5 (M + H). |
| | | 3-((4-(1′-(4-chloro-7- | |
| | | cyclopentyl-5-oxo-5,7- | |
| | | dihydrobenzo[4,5]imidazo[1,2- | |
| | | a]quinazolin-9-yl)-[1,4′- | |
| | | bipiperidin]-4-yl)-3- | |
| | | fluorophenyl)amino)piperidine- | |
| | | 2,6-dione | |
| 39 | Int-18 & 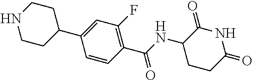 [SMILES: O=C1CCC(NC(=O)c2ccc(C3CCNCC3)cc2F)C(=O)N1] | 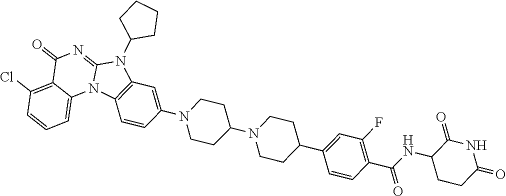 [SMILES: O=C1CCC(NC(=O)c2ccc(C3CCN(C4CCN(c5ccc6c(c5)n(C5CCCC5)c5nc(=O)c7c(Cl)cccc7n65)CC4)CC3)cc2F)C(=O)N1] | ¹H NMR (400 MHz, DMSO-d₆): δ 10.80 (s, 1H), 8.37 (d, J = 8.0 Hz, 1H), 8.20 (d, J = 8.2 Hz, 1H), 7.77 (t, J = 16.0 Hz, 1H), 7.60-7.46 (m, 2H), 7.21-7.15 (m, 4H), 7.00 (d, J = 6.8 Hz, 1H), 5.25-5.21 (m, 1H), 4.41-4.39 (m, 1H), 3.96-3.66 (m, 3H), 2.82-2.79 (m, 4H), 2.57-2.50 (m, 3H), 2.34-2.22 (m, 4H), 2.20- 2.18 (m, 2H), 2.09-1.98 (m, 10H), 1.75-1.73 (m, 4H); LC-MS: m/z 724.5 (M + H). |
| | | 4-(1′-(4-chloro-7-cyclopentyl-5- | |
| | | oxo-5,7- | |
| | | dihydrobenzo[4,5]imidazo[1,2- | |
| | | a]quinazolin-9-yl)-[1,4′- | |
| | | bipiperidin]-4-yl)-N-(2,6- | |
| | | dioxopiperidin-3-yl)-2- | |
| | | fluorobenzamide | |
| 40 | Int-7 & 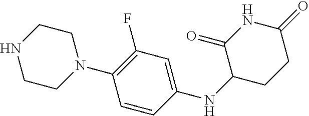 [SMILES: O=C1CCC(Nc2ccc(N3CCNCC3)c(F)c2)C(=O)N1] | 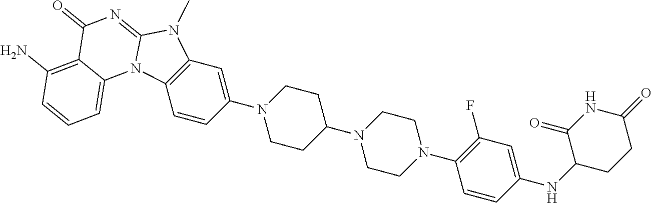 [SMILES: Cn1c2cc(N3CCC(N4CCN(c5ccc(NC6CCC(=O)NC6=O)cc5F)CC4)CC3)ccc2n2c3cccc(N)c3c(=O)nc12] | ¹H NMR (400 MHz, DMSO-d₆): δ 10.79 (s, 1H), 9.50 (bs, 1H), 8.11 (d, J = 8.0 Hz, 1H), 7.44 (t, J = 16.0 Hz, 1H), 7.30 (d, J = 8.2 Hz, 1H), 7.25 (s, 1H), 7.00 (d, J = 8.8 Hz, 1H), 6.91 (t, J = 9.2 Hz, 1H), 6.68 (d, J = 8.4 Hz, 1H), 6.58 (d, J = 8.0 Hz, 1H), 6.54 (d, J = 4.0 Hz, 1H), 4.32-4.29 (m, 1H), 4.06 (d, J = 12.0 Hz, 2H), 3.67-3.62 (m, 5H), 3.51-3.50 (m, 1H), 3.34-3.25 (m, 3H), 3.02-2.96 (m, 2H), 2.86-2.68 |
| | | 3-((4-(4-(1-(4-amino-7-methyl- | (m, 3H), 2.62-2.60 (m, 1H), 2.59- |
| | | 5-oxo-5,7- | 2.55 (m, 1H), 2.24-2.21 (m, 2H), |
| | | dihydrobenzo[4,5]imidazo[1,2- | 2.10-2.06 (m, 1H), 1.89-1.76 (m, |
| | | a]quinazolin-9-yl)piperidin-4- | 3H); LC-MS: m/z 652.4 (M + H). |
| | | yl)piperazin-1-yl)-3- | |
| | | fluorophenyl)amino)piperidine- | |
| | | 2,6-dione | |
| 41 | Int-7 & 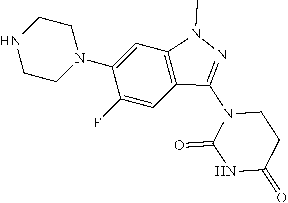 | 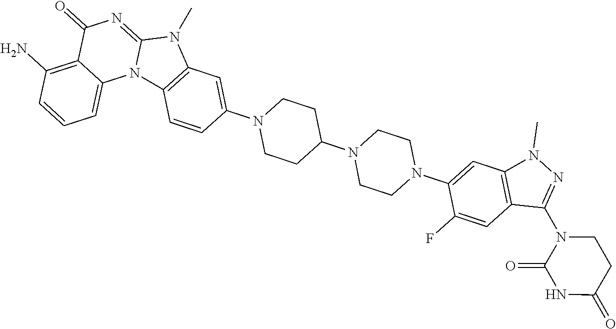 [SMILES: Cn1nc(N2CCC(=O)NC2=O)c2cc(F)c(N3CCN(C4CCN(c5ccc6c(c5)n(C)c5nc(=O)c7c(N)cccc7n65)CC4)CC3)cc21] | ¹H NMR (400 MHz, DMSO-d₆): δ 10.55 (s, 1H), 9.48 (bs, 1H), 8.10 (d, J = 9.2 Hz, 1H), 7.46-7.40 (m, 2H), 7.30-7.23 (m, 3H), 7.00 (d, J = 8.0 Hz, 1H), 6.66 (d, J = 8.0 Hz, 1H), 4.09-4.06 (m, 2H), 3.99 (s, 3H), 3.90 (t, J = 6.8 Hz, 2H), 3.73- 3.65 (m, 8H), 3.17-3.12 (m, 2H), 2.89-2.82 (m, 2H), 2.75 (t, J = 6.4 Hz, 2H), 2.68-2.66 (m, 2H), 2.27- 2.23 (m, 2H), 2.01-1.99 (m, 1H), 1.85-1.82 (m, 1H); LC-MS: m/z 692.3 (M + H). |
| | | 1-(6-(4-(1-(4-amino-7-methyl-5- | |
| | | oxo-5,7- | |
| | | dihydrobenzo[4,5]imidazo[1,2- | |
| | | a]quinazolin-9-yl)piperidin-4- | |
| | | yl)piperazin-1-yl)-5-fluoro-1- | |
| | | methyl-1H-indazol-3- | |
| | | yl)dihydropyrimidine- | |
| | | 2,4(1H,3H)-dione | |
| 42 | Int-7 & 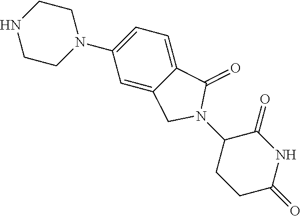 [SMILES: O=C1CCC(N2Cc3cc(N4CCNCC4)ccc3C2=O)C(=O)N1] | 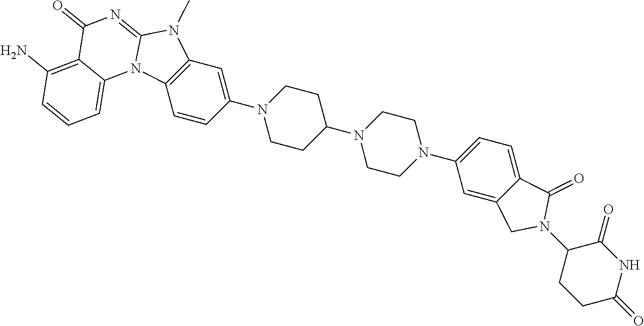 [SMILES: Cn1c2cc(N3CCC(N4CCN(c5ccc6c(c5)CN(C5CCC(=O)NC5=O)C6=O)CC4)CC3)ccc2n2c3cccc(N)c3c(=O)nc12] | ¹H NMR (400 MHz, DMSO-d₆): δ 10.96 (s, 1H), 9.53 (bs, 1H), 8.10 (d, J= 9.2 Hz, 1H), 7.61 (d, J = 8.4 Hz, 1H), 7.42 (t, J = 8.0 Hz, 1H), 7.29 (d, J = 8.4 Hz, 1H), 7.24 (s, 1H), 7.20-7.17 (m, 2H), 7.00 (d, J = 2.4 Hz, 1H), 6.66 (d, J = 7.6 Hz, 1H), 5.10-5.05 (m, 1H), 4.39-4.35 (m, 1H), 4.27-4.23 (m, 1H), 4.10- 4.05 (m, 4H), 3.70 (d, J = 9.0 Hz, 2H), 3.60 (s, 3H), 3.52 (bs, 2H), 3.27-3.24 (m, 2H), 3.15-3.12 (m, 2H), 2.87-2.81 (m, 2H), 2.66-2.61 (m, 1H), 2.34-2.31 (m, 1H), 2.32- 2.21 (m, 2H), 1.99-1.98 (m, 1H), 1.82-1.79 (m, 2H); LC-MS: m/z 674.7 (M + H). |
| | | 3-(5-(4-(1-(4-amino-7-methyl-5- | |
| | | oxo-5,7- | |
| | | dihydrobenzo[4,5]imidazo[1,2- | |
| | | a]quinazolin-9-yl)piperidin-4- | |
| | | yl)piperazin-1-yl)-1- | |
| | | oxoisoindolin-2-yl)piperidine- | |
| | | 2,6-dione | |
| 43 | Int-16 & 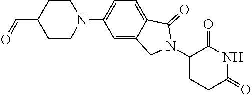 [SMILES: O=CC1CCN(c2ccc3c(c2)CN(C2CCC(=O)NC2=O)C3=O)CC1] | 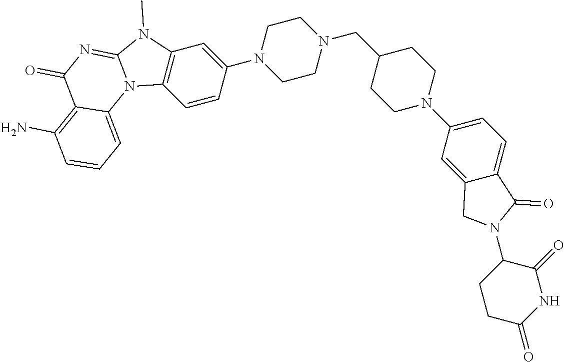 [SMILES: Cn1c2cc(N3CCN(CC4CCN(c5ccc6c(c5)CN(C5CCC(=O)NC5=O)C6=O)CC4)CC3)ccc2n2c3cccc(N)c3c(=O)nc12] | ¹H NMR (400 MHz, DMSO-d₆): δ 10.94 (s, 1H), 8.08 (d, J = 9.2 Hz, 1H), 7.51 (d, J = 9.2 Hz, 1H), 7.41 (t, J = 8.0 Hz, 1H), 7.28 (d, J = 8.0 Hz, 1H), 7.18 (bs, 1H), 7.05 (t, J = 8.0 Hz, 2H), 6.94 (d, J = 7.6 Hz, 1H), 6.65 (d, J = 8.0 Hz, 1H), 5.07- 5.02 (m, 1H), 4.72-4.68 (m, 1H), 4.35-4.29 (m, 1H), 4.22-4.18 (m, 1H), 3.92-3.88 (m, 2H), 3.63 (s, 3H), 3.52 (d, J = 2.0 Hz, 2H), 3.27 (bs, 4H), 2.87-2.86 (m, 3H), 2.67- 2.57 (m, 4H), 2.27-2.22 (m, 3H), 1.99-1.96 (m, 1H), 1.83 (d, J = 11.2 Hz, 3H); LC-MS: m/z 688.3 (M + H). |
| | | 3-(5-(4-((4-(4-amino-7-methyl-5- | |
| | | oxo-5,7- | |
| | | dihydrobenzo[4,5]imidazo[1,2- | |
| | | a]quinazolin-9-yl)piperazin-1- | |
| | | yl)methyl)piperidin-1-yl)-1- | |
| | | oxoisoindolin-2-yl)piperidine-2,6- | |
| | | dione | |
| 44 | Int-6 & Int-1 | 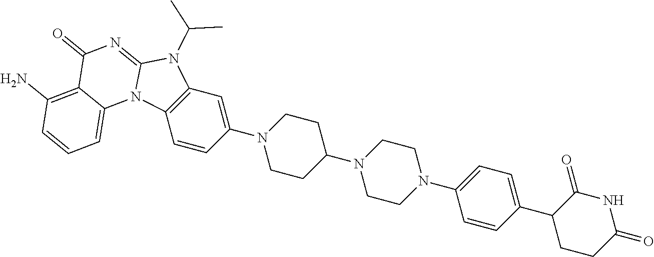 [SMILES: CC(C)n1c2cc(N3CCC(N4CCN(c5ccc(C6CCC(=O)NC6=O)cc5)CC4)CC3)ccc2n2c3cccc(N)c3c(=O)nc12] | LC-MS: m/z 647.3 (M + H), HPLC: purity: 98.8 %. |
| | | 3-(4-(4-(1-(4-amino-7-isopropyl-5- | |
| | | oxo-5,7- | |
| | | dihydrobenzo[4,5]imidazo[1,2- | |
| | | a]quinazolin-9-yl)piperidin-4- | |
| | | yl)piperazin-1- | |
| | | yl)phenyl)piperidine-2,6-dione | |
| 45 | Int-16 & 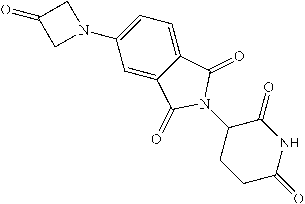 [SMILES: O=C1CN(c2ccc3c(c2)C(=O)N(C2CCC(=O)NC2=O)C3=O)C1] | 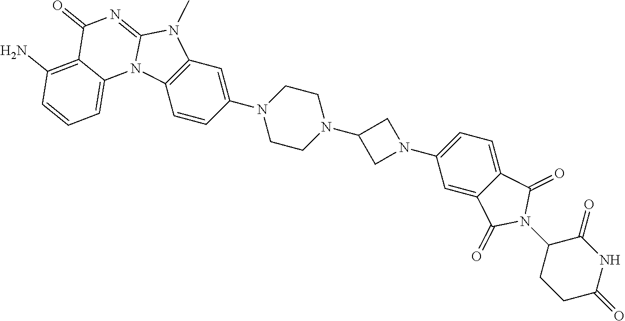 [SMILES: Cn1c2cc(N3CCN(C4CN(c5ccc6c(c5)C(=O)N(C5CCC(=O)NC5=O)C6=O)C4)CC3)ccc2n2c3cccc(N)c3c(=O)nc12] | LC-MS: m/z 660.2 (M + H). HPLC: purity: 97.55%. |
| | | 5-(3-(4-(4-amino-7-methyl-5-oxo- | |
| | | 5,7-dihydrobenzo[4,5]imidazo[1,2- | |
| | | a]quinazolin-9-yl)piperazin-1- | |
| | | yl)azetidin-1-yl)-2-(2,6- | |
| | | dioxopiperidin-3-yl)isoindoline- | |
| | | 1,3-dione | |
| 46 | Int-19 & 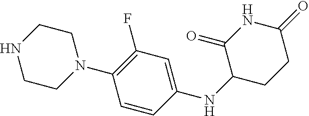 [SMILES: O=C1CCC(Nc2ccc(N3CCNCC3)c(F)c2)C(=O)N1] | 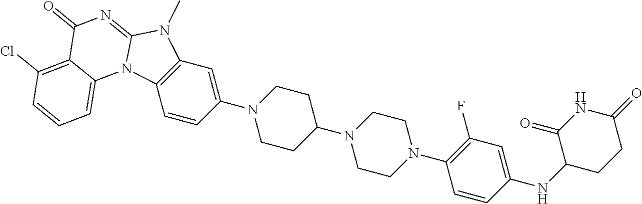 [SMILES: Cn1c2cc(N3CCC(N4CCN(c5ccc(NC6CCC(=O)NC6=O)cc5F)CC4)CC3)ccc2n2c3cccc(Cl)c3c(=O)nc12] | ¹H NMR (400 MHz, DMSO-d₆): δ 8.35 (d, J = 8.8 Hz, 1H), 8.18 (d, J = 9.2 Hz, 1H), 7.77 (t, J = 8.4 Hz, 1H), 7.55 (d, J = 7.6 Hz, 1H), 7.21 (s, 1H), 6.97 (d, J = 8.0 Hz, 1H), 6.89-6.81 (m, 1H), 6.53 (d, J = 15.6 Hz, 1H), 6.43 (d, J = 8.0 Hz, 1H), 5.81 (bs, 1H), 4.29-4.21 (m, 1H), 3.93-3.85 (m, 2H), 3.64 (s, 3H), 2.89-2.75 (m, 4H), 2.71-2.62 (m, 4H), 2.61-2.59 (m, 1H), 2.55- 2.51 (m, 1H), 2.49-2.43 (m, 3H), 2.12-2.08 (m, 1H), 1.99-1.91 (m, |
| | | 3-((4-(4-(1-(4-chloro-7-methyl-5- | 2H), 1.88-1.81 (m, 2H); LC-MS: |
| | | oxo-5,7- | m/z 671.2 (M + H). |
| | | dihydrobenzo[4,5]imidazo[1,2- | |
| | | a]quinazolin-9-yl)piperidin-4- | |
| | | yl)piperazin-1-yl)-3- | |
| | | fluorophenyl)amino)piperidine-2,6- | |
| | | dione | |
| 47 | Int-20 & 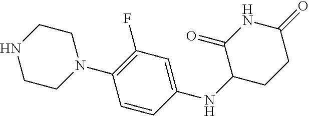 [SMILES: O=C1CCC(Nc2ccc(N3CCNCC3)c(F)c2)C(=O)N1] | 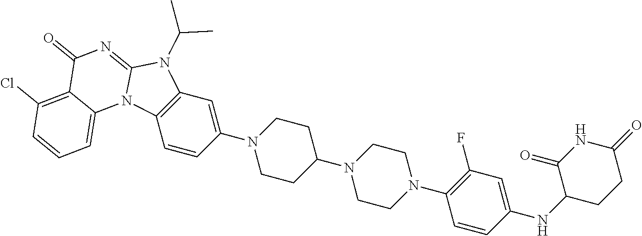 [SMILES: CC(C)n1c2cc(N3CCC(N4CCN(c5ccc(NC6CCC(=O)NC6=O)cc5F)CC4)CC3)ccc2n2c3cccc(Cl)c3c(=O)nc12] | ¹H NMR (400 MHz, DMSO-d₆): δ 10.85 (s, 1H), 8.36 (d, J = 8.8 Hz, 1H), 8.18 (d, J = 9.2 Hz, 1H), 7.77 (t, J = 8.0 Hz, 1H), 7.55 (d, J = 8.0 Hz, 1H), 7.22 (d, J = 2.0 Hz, 1H), 6.97 (dd, J = 11.2 & 2.0 Hz, 1H), 6.83 (t, J = 9.2 Hz, 1H), 6.51 (dd, J = 18.8 & 2.0 Hz, 1H), 6.42 (dd, J = 8.8 & 2.0 Hz, 1H), 5.79 (d, J = 7.6 Hz, 1H), 5.16-5.09 (m, 1H), 4.24-4.18 (m, 1H), 3.88 (d, J = 12.4 Hz, 1H), 2.89 (bs, 4H), 2.82- 2.75 (m, 2H), 2.68 (bs, 4H), 2.58- 2.53 (m, 4H), 2.47-2.43 (m, 2H), 2.11-2.08 (m, 1H), 1.93 (d, J = |
| | | 3-((4-(4-(1-(4-chloro-7-isopropyl-5- | 12.0 Hz, 2H), 1.86-1.81 (m, 1H), |
| | | oxo-5,7- | 1.61 (d, J = 6.8 Hz, 6H); LC-MS: |
| | | dihydrobenzo[4,5]imidazo[1,2- | m/z 699.29 (M + H). |
| | | a]quinazolin-9-yl)piperidin-4- | |
| | | yl)piperazin-1-yl)-3- | |
| | | fluorophenyl)amino)piperidine-2,6- | |
| | | dione | |
| 48 | Int-20 & 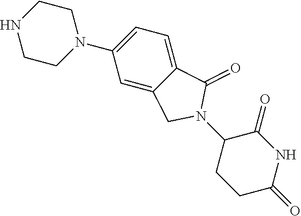 [SMILES: O=C1CCC(N2Cc3cc(N4CCNCC4)ccc3C2=O)C(=O)N1] | 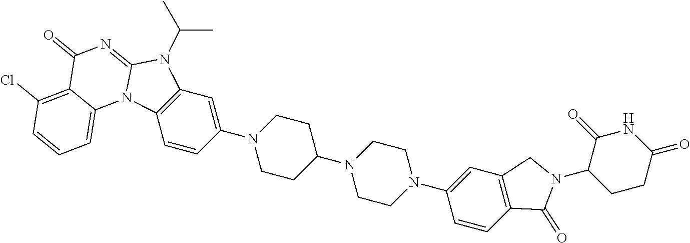 [SMILES: CC(C)n1c2cc(N3CCC(N4CCN(c5ccc6c(c5)CN(C5CCC(=O)NC5=O)C6=O)CC4)CC3)ccc2n2c3cccc(Cl)c3c(=O)nc12] | ¹H NMR (400 MHz, DMSO-d₆): δ 10.95 (s, 1H), 8.36 (d, J = 8.4 Hz, 1H), 8.18 (d, J = 9.2 Hz, 1H), 7.77 (t, J = 8.0 Hz, 1H), 7.54 (t, J = 7.2 Hz, 2H), 7.25 (d, J = 2.0 Hz, 1H), 7.07 (d, J = 7.6 Hz, 2H), 6.98 (dd, J = 11.2 & 4.2 Hz, 1H), 5.13 (t, J = 6.8 Hz, 1H), 5.07-5.03 (m, 1H), 4.35 (d, J = 16.8 Hz, 1H), 4.20 (d, J = 16.8 Hz, 1H), 3.90 (d, J = 12.0 Hz, 2H), 3.49 (bs, 4H), 2.84-2.83 (m, 1H), 2.81 (t, J = 10.8 Hz, 1H), 2.71 (bs, 4H), 2.63-2.52 (m, 2H), 2.45-2.32 (m, 4H), 2.01-1.90 (m, 3H), 1.61 (d, J = 7.0 Hz, 6H); LC- |
| | | 3-(5-(4-(1-(4-chloro-7-isopropyl-5- | MS: m/z 721.3 (M + H). |
| | | oxo-5,7- | |
| | | dihydrobenzo[4,5]imidazo[1,2- | |
| | | a]quinazolin-9-yl)piperidin-4- | |
| | | yl)piperazin-1-yl)-1-oxoisoindolin- | |
| | | 2-yl)piperidine-2,6-dione | |

### Example-2: Synthesis of 3-((4-(4-(4-(4-amino-7-methyl-5-oxo-5,7-dihydrobenzo[4,5]imidazo[1,2-a]quinazolin-9-yl)phenyl) piperazin-1-yl)-3-fluorophenyl)amino)piperidine-2,6-dione (Compound-49)

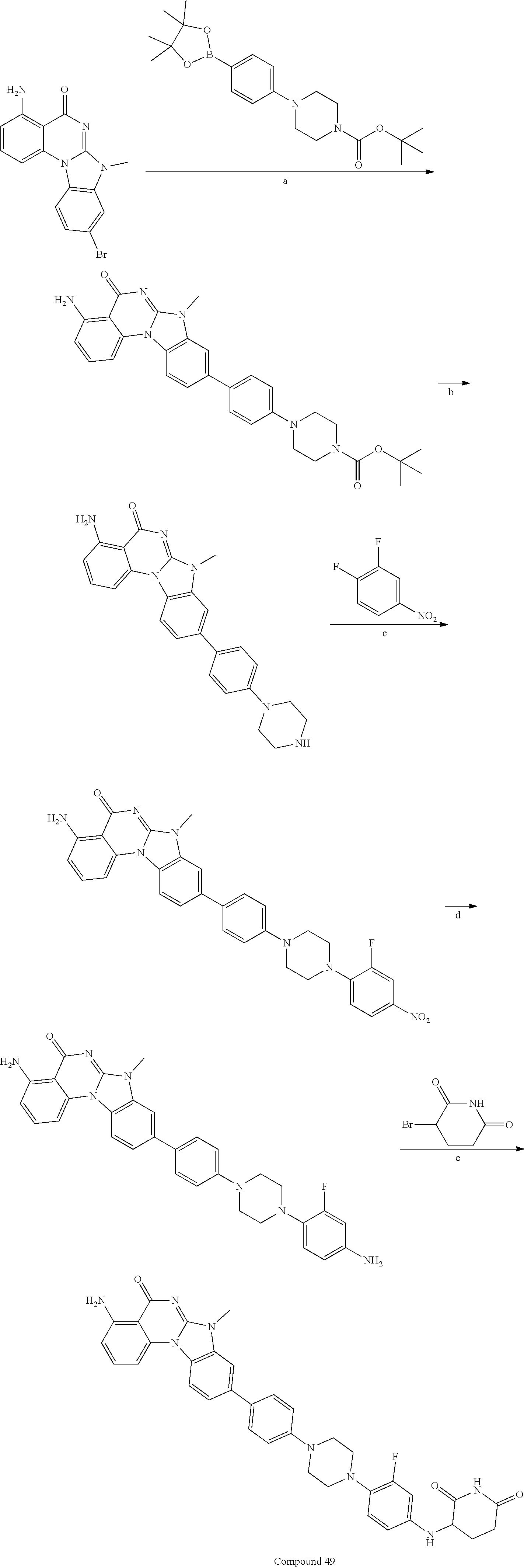

### Step a: Synthesis of tert-butyl 4-(4-(4-amino-7-methyl-5-oxo-5,7-dihydrobenzo[4,5]imidazo[1,2-a]quinazolin-9-yl)phenyl) piperazine-1-carboxylate

4-amino-9-bromo-7-methylbenzo[4,5]imidazo[1,2-a]quinazolin-5(7H)-one (0.500 g, 1.457 mmol), tert-butyl 4-(4-(4,4,5,5-tetramethyl-1,3,2-dioxaborolan-2-yl)phenyl) piperazine-1-carboxylate (0.679 g, 1.748 mmol) and potassium acetate (0.357 g, 3.630 mmol) was dissolved in 1,4-dioxane (7 mL) and water (3 ml), and purged with nitrogen for 10 minutes to obtain a solution. After purging, Pd(dppf)Cl₂.DCM (0.119 g, 0.140 mmol) was added in the earlier solution to obtain first reaction mixture. Then, this first reaction mixture was heated to 100° C. for 16 h to obtain the second reaction mixture. After completion of the reaction (monitored by TLC), the second reaction mixture was diluted with EtOAc and washed with water, brine, and dried over anhydrous sodium sulphate. Then, combined organic layer was concentrated under vacuum to give the residue which was purified by combi flash column chromatography using 2-3% methanol in DCM as eluent to afford the title compound as yellow solid (0.550 g, 72%). ¹H NMR (400 MHz, DMSO-d₆): δ 8.25 (d, J=8.4 Hz, 1H), 7.85 (d, J=1.6 Hz, 1H), 7.70 (d, J=8.6 Hz, 2H), 7.58 (dd, J=8.8, 2.0 Hz, 1H), 7.44 (t, J=8.8 Hz, 1H), 7.36 (d, J=7.6 Hz, 1H), 7.07 (d, J=8.4 Hz, 2H), 6.68 (d, J=7.6 Hz, 1H), 3.71 (s, 3H), 3.49 (t, J=4.8 Hz, 4H), 3.18 (t, J=5.2 Hz, 4H), 1.44 (s, 9H); LC-MS: m/z 525.2 (M+H).

### Step-b: Synthesis 4-amino-7-methyl-9-(4-(piperazin-1-yl)phenyl)benzo[4,5]imidazo[1,2-a]quinazolin-5(7H)-one

tert-butyl 4-(4-(4-amino-7-methyl-5-oxo-5,7-dihydrobenzo[4,5]imidazo[1,2-a]quinazolin-9-yl)phenyl) piperazine-1-carboxylate (0.550 g, 1.048 mmol) was dissolved in DCM (5 mL) to obtain a solution. Then, 4N dioxane hydrochloride (10 mL) was added into the reactant solution at 0° C. to obtain first reaction mixture and then slowly the first reaction mixture temperature was brought into RT and stirred for 4 h to obtain second reaction mixture. After completion of reaction, the second reaction mixture was evaporated under reduced pressure to afford the residue, which was later washed with diethyl ether and dried under vacuum to afford the title compound as yellowish solid (0.550 g, crude). ¹H NMR (400 MHz, DMSO-d₆): δ 9.46 (s, 2H), 8.43 (d, J=8.8 Hz, 1H), 8.06 (d, J=1.6 Hz, 1H), 7.79-7.73 (m, 3H), 7.58-7.51 (m, 2H), 7.14 (d, J=8.8 Hz, 2H), 6.86 (bs, 2H), 6.84 (d, J=8.0 Hz, 1H), 3.88 (s, 3H), 3.49 (t, J=4.8 Hz, 4H), 3.17 (t, J=bs, 4H): LCMS: m/z 425.2 (M+H).

### Step-c: Synthesis of 4-amino-9-(4-(4-(2-fluoro-4-nitrophenyl) piperazin-1-yl)phenyl)-7-methylbenzo[4,5]imidazo[1,2-a]quinazolin-5(7H)-one

4-amino-7-methyl-9-(4-(piperazin-1-yl)phenyl)benzo[4,5]imidazo[1,2-a]quinazolin-5(7H)-one (0.300 g, 0.707 mmol) was dissolved in DMF (50 mL) to obtain a solution. 1,2-difluoro-4-nitrobenzene (0.112 g, 0.707 mmol) and DiPEA (0.215 g, 2.120 mmol) was added to reactant solution at RT and stirred for 16 h at RT to obtain first reaction mixture. After completion of reaction (monitored by TLC), the first reaction mixture was quenched with cold water and stirred for 1 h. Solid residue formed, and it was filtered and dried under vacuum to afford the title compounds as yellow solid (0.220 g, crude). ¹H NMR (400 MHz, DMSO-d₆): δ 8.29 (d, J=8.8 Hz, 1H), 8.09 (d, J=2.4 Hz, 1H), 8.06 (s, 1H), 7.89 (s, 1H), 7.75 (d, J=8.8 Hz, 2H), 7.62 (d, J=8.8 Hz, 1H), 7.45 (t, J=6.8 Hz, 1H), 7.39 (d, J=8.7 Hz, 1H), 7.20 (d, J=2.0 Hz, 1H), 7.15 (d, J=8.8 Hz, 2H), 6.69 (d, J=8.4 Hz, 1H), 3.73 (s, 3H), 3.49 (d, J=5.6 Hz, 4H), 3.43 (bs, 4H); LC-MS: m/z 564.2 (M+H).

### Step-d: Synthesis of 4-amino-9-(4-(4-(4-amino-2-fluorophenyl) piperazin-1-yl)phenyl)-7-methylbenzo[4,5]imidazo[1,2-a]quinazolin-5(7H)-one

4-amino-9-(4-(4-(2-fluoro-4-nitrophenyl) piperazin-1-yl)phenyl)-7-methylbenzo[4,5]imidazo[1,2-a]quinazolin-5(7H)-one (0.220 g, 0.399 mmol) was dissolved in EtOH (5 mL) and water (5 mL) mixture to obtain a solution. NH₄Cl (0.427 g, 7.980 mmol) was added in the reactant solution followed by the addition of Iron powder (0.067 g, 1.190 mmol) portion wise to obtain first reaction mixture and first reaction mixture was stirred at RT for 10 mins. After completion of the reaction (monitored by TLC), reaction mixture was passed through celite bed and thoroughly washed with EtOAc. Filtrate was washed with Sat. bicarbonate solution, water and brine solution and dried over Na₂SO₄. Collective organic layer was concentrated to afford the title compound as brick red sticky solid (0.170 g, crude); LC-MS: m/z 534.2 (M+H).

### Step-e: Synthesis of 3-((4-(4-(4-(4-amino-7-methyl-5-oxo-5,7-dihydrobenzo[4,5]imidazo[1,2-a]quinazolin-9-yl)phenyl) piperazin-1-yl)-3-fluorophenyl)amino)piperidine-2,6-dione

4-amino-9-(4-(4-(4-amino-2-fluorophenyl) piperazin-1-yl)phenyl)-7-methylbenzo[4,5]imidazo[1,2-a]quinazolin-5(7H)-one (0.130 g, 0.244 mmol) was dissolved in 1,4-dioxane (2 mL) to obtain a solution. 3-bromopiperidine-2,6-dione (0.047 g, 0.244 mmol) and DIPEA (0.095 g, 0.730 mmol) was added to the reactant solution at RT and heated to 110° C. for 16 h to obtain first reaction mixture. After completion of the reaction (monitored by TLC), the reaction mixture was concentrated under vacuum to obtained crude material. Crude material was first purified by combi flash column chromatography using 10% methanol in DCM as eluent, followed by perp HPLC (using Column: Luna, omega ps, C18, flow Rate 15.0 ml/minute, Mobile Phase-A: 0.05% TFA in Water, Mobile Phase-B: Acetonitrile) purification to afford the title compound as TFA salt (yellow solid, 0.010 g, 6.4%). ¹H NMR (400 MHz, DMSO-d₆): δ 10.79 (s, 1H), 8.30 (d, J=8.8 Hz, 1H), 7.90 (d, J=1.6 Hz, 1H), 7.73 (d, J=8.8 Hz, 2H), 7.63 (dd, J=8.8, 1.6 Hz, 1H), 7.48-7.39 (m, 2H), 7.13 (d, J=8.8 Hz, 2H), 6.92 (t, J=9.6 Hz, 1H), 6.70 (d, J=7.6 Hz, 1H), 6.57 (dd, J=14.8, 1.2 Hz, 1H), 6.46 (dd, J=8.4, 2.0 Hz, 1H), 4.31-4.27 (m, 1H), 3.74 (s, 3H), 3.37 (bs, 4H), 3.06 (bs, 4H), 2.80-2.70 (m, 1H), 2.58-2.55 (m, 1H), 2.12-2.07 (m, 1H), 1.92-1.84 (m, 1H); LC-MS: m/z 645.2 (M+H).

### Example 3: Synthesis of 3-((4-(4-(4-(4-chloro-7-isopropyl-5-oxo-5,7-dihydrobenzo[4,5]imidazo[1,2-a]quinazolin-9-yl)phenyl) piperazin-1-yl)-3-fluorophenyl)amino)piperidine-2,6-dione.TFA (Compound 50)

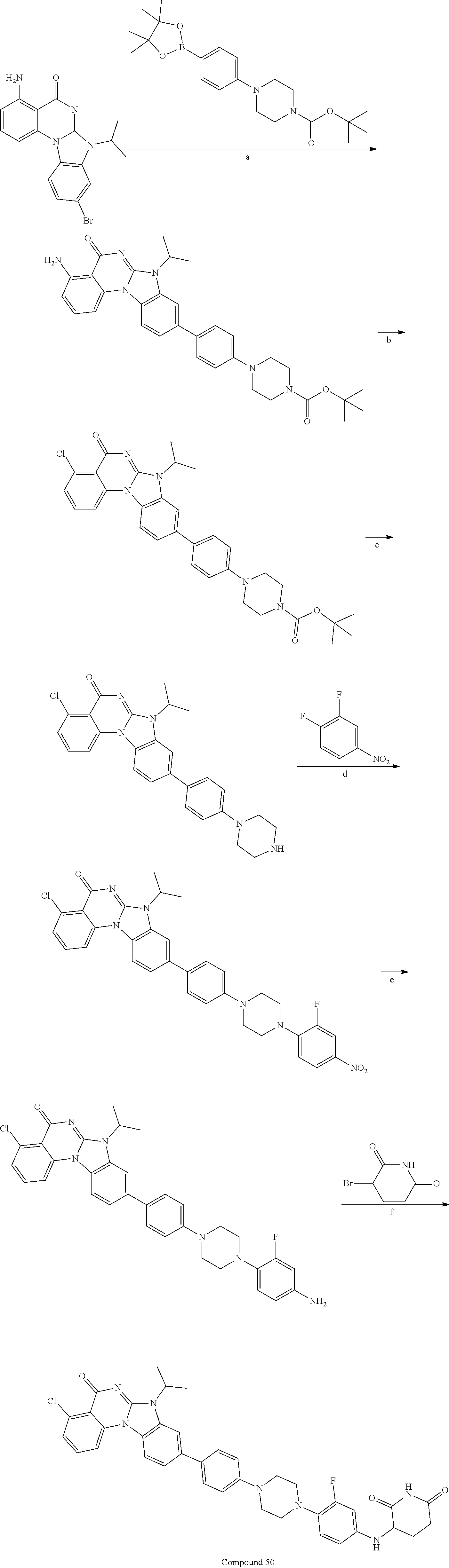

Compound-50 was prepared by procedure similar to the one described in Example-2 with appropriate variations in reactants, quantities of reagents, protections and deprotections, solvents and reaction conditions. ¹H NMR (400 MHz, DMSO-d6): δ 10.79 (s, 1H), 8.48 (d, J=8.4 Hz, 1H), 8.31 (d, J=8.8 Hz, 1H), 7.93 (d, J=1.2 Hz, 1H), 7.81 (t, J=8.0 Hz, 1H), 7.73 (d, J=8.8 Hz, 2H), 7.65-7.59 (m, 2H), 7.13 (d, J=8.8 Hz, 2H), 6.93 (t, J=9.6 Hz, 1H), 6.57 (dd, J=15.2, 1.2 Hz, 1H), 6.46 (dd, J=8.4, 2.4 Hz, 1H), 5.25-5.18 (m, 1H), 4.31-4.27 (m, 1H), 3.37 (bs, 4H), 3.06 (bs, 4H), 2.81-2.70 (m, 1H), 2.59-2.55 (m, 1H), 2.13-2.07 (m, 1H), 1.93-1.82 (m, 1H), 1.66 (d, J=7.2 Hz, 6H); LCMS m/z 692.4 (M+H).

### Example-P1: Determination of Anti Proliferative Activity of Compounds in Cell Lines SK-MEL-5 by Cell Titer Glo® (Promega) Assay

Cells were seeded into 96-well plates and the plate was incubated in 37 degrees incubator overnight. The next day, compounds were diluted 3-fold to cover 9-point concentration range in DMSO. Intermediate plate dilution was prepared in media followed with compound treatment in cells. Retreatment of cells with compound dilutions was performed on Day 4 and assay was terminated on day 8 for SK-MEL-5 cells using CellTiter-Glo and the plate was kept on orbital shaker for 20 minutes at RT. Assay was terminated on day 6 for RERF-LC-A1 without a compound retreatment. Luminescence signal was recorded on VICTOR³ instrument. Percent inhibition of proliferation was calculated at each concentration and plotted against the compound concentration. EC₅₀ value was calculated using GraphPad® software.

### Example-P2: Determination of Anti Proliferative Activity of Compounds in Cell Lines MV-4-11 by Cell Titer Glo® (Promega) Assay

MV-4-11 (CRL-9591™) cells were seeded in 96 well plate flat black clear bottom plates (Corning, Cat. No 3904) using complete DMEM Medium. Next day, selected compounds of the present invention were added to cells from 10 mM stocks made in DMSO (Sigma Cat no. D2650). Each concentration of compound was tested in triplicate with DMSO concentration at a final percentage not exceeding 0.3 in the cells. After the incubation of MV-4-11 cells with compound for 3 days; assay was terminated using 100 μl of CellTiter Glo® reagent (Promega, Cat. no G7572). CellTiter-Glo® Luminescent reagent determines the number of viable cells based on quantitation of ATP present which is an indicator of cell number and metabolic activity. Luminescence readings were taken in Victor-5 instrument. Percent inhibition of proliferation was calculated using formula, % inhibition=100−(luminescence value of test/luminescence value of DMSO control)*100. DMSO control (0%)=Cells in complete media with DMSO; blank=Media alone containing DMSO. EC₅₀ was calculated using graph pad prism software.

Selected compounds of the present invention were screened in the above-mentioned assay procedures for determination of EC₅₀ (MV-4-11 & SK-MEL-5) values and the results are summarized into groups A, B and C in the below table. Herein the group “A” refers to EC₅₀ values lower than 10 nM, group “B” refers to EC₅₀ values between 10.01 nM to 100 nM (both inclusive), group “C” refers to EC₅₀ values greater than 100.

| | |
|---|---|
| Comp | EC50 (nM) |

| No. | MV-4-11 | SK-MEL-5 |
|---|---|---|
| 1 | A | A |
| 2 | A | A |
| 3 | A | A |
| 4 | A | A |
| 5 | C | C |
| 6 | C | A |
| 7 | A | A |
| 8 | A | A |
| 9 | A | A |
| 10 | C | C |
| 11 | C | C |
| 12 | C | C |
| 13 | C | C |
| 14 | A | A |
| 15 | A | A |
| 16 | A | A |
| 17 | A | A |
| 18 | A | A |
| 19 | C | C |
| 20 | C | B |
| 21 | C | B |
| 22 | A | A |
| 23 | A | A |
| 24 | C | C |
| 25 | B | A |
| 26 | C | B |
| 27 | A | A |
| 28 | A | A |
| 29 | A | A |
| 30 | C | A |
| 31 | C | C |
| 32 | C | C |
| 33 | C | C |
| 34 | C | C |
| 35 | C | A |
| 36 | C | A |
| 37 | C | C |
| 38 | A | A |
| 39 | C | C |
| 40 | C | C |
| 41 | C | C |
| 42 | C | C |
| 43 | C | C |
| 44 | C | C |
| 45 | C | C |
| 46 | C | A |
| 47 | C | A |
| 48 | C | A |
| 49 | C | C |
| 50 | C | C |

### Example-P3: Determination of SMARCA2 and SMARCA4 Degradation in MV-4-11 Cells by Western Blot

MV-4-11 (CRL-9591™) cells were plated in 6 well plates using complete Iscove's Modified Dulbecco's Medium. On the third day, compounds of present invention were added to cells from 10 mM stocks made in DMSO (Sigma Cat no. D2650). Each concentration of compound was tested with DMSO not exceeding final percentage of 0.1 in the cells. Cells were incubated with the compound for 16 hours followed by harvesting with 1×RIPA lysis buffer (Thermo Fischer, catalogue number #89900) containing protease inhibitor cocktail (Sigma catalogue number #P-8340). Equal amount of protein was loaded on SDS PAGE gel for electrophoresis. Western blot was carried out for detection of either SMARCA2 (Cell signalling technologies, catalogue number #11966) or SMARCA4 antibody (Cell signalling technologies, catalogue number #52251). Beta-Tubulin antibody (Cell signalling technologies, catalogue number #86298) was used as loading control. Percentage of SMARCA2 or SMARCA4 degradation was calculated using formula % Degradation=100−(normalized band intensity in treated sample/normalized band intensity in DMSO sample)*100. Here, “ND” refers to Not determined.

The results are given below.

| | |
|---|---|
| | Percent degradation |
| Comp | (at 100 nM) |

| No. | SMARCA2 | SMARCA4 |
|---|---|---|
| 1 | 100 | 98 |
| 2 | 100 | 100 |
| 3 | 100 | 99 |
| 4 | 90 | 97 |
| 6 | 96 | 90 |
| 7 | 99 | 99 |
| 8 | 92 | 99 |
| 9 | 90 | 99 |
| 14 | 100 | 94 |
| 15 | 96 | 95 |
| 16 | 99 | 100 |
| 17 | 68 | 24 |
| 18 | 98 | 95 |
| 20 | 80 | 81 |
| 21 | 86 | 75 |
| 22 | 89 | 56 |
| 23 | 99 | 98 |
| 25 | 67 | 33 |
| 26 | 68 | 33 |
| 29 | 96 | 97 |
| 30 | 90 | 64 |
| 32 | 59 | 38 |
| 35 | 98 | 91 |
| 36 | 99 | 97 |
| 38 | 98 | 98 |
| 40 | 48 | ND |
| 44 | 16 | ND |
| 45 | 21 | ND |

### INCORPORATION BY REFERENCE

All publications and patents mentioned herein are hereby incorporated by reference in their entirety as if each individual publication or patent were specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents and the specification, along with such variations.

1: A compound of formula (I): 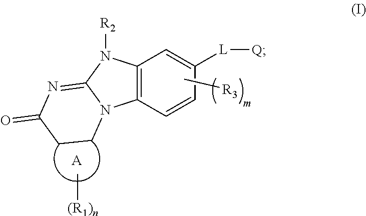
or a pharmaceutically acceptable salt or a stereoisomer or a tautomer thereof;
wherein,
A is phenyl or pyridyl;
each R₁ and R₃ independently is halogen, (C₁-C₆)alkyl, (C₁-C₆)alkoxy, amino, halo(C₁-C₆)alkyl, hydroxy, 3- to 8-membered cycloalkyl, hydroxy (C₁-C₆)alkyl or cyano;
R₂ is hydrogen, halogen, hydroxy, (C₁-C₆)alkyl or 3- to 8-membered cycloalkyl; wherein the alkyl and cycloalkyl is substituted with 0, 1, 2 or 3 substituents, independently, selected from hydroxy, halogen and (C₁-C₆)alkoxy;
L is 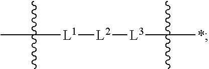
wherein asterisk mark represents the point of attachment with Q;
L¹ and L³ independently is a bond, (C₁-C₆)alkylenyl, 6- to 10-membered arylenyl or 4- to 6-membered heterocycloalkylenyl; wherein the alkylenyl, arylenyl and heterocycloalkylenyl, at each occurrence, independently is substituted with 0, 1, 2, 3 or 4 of R₇;
L² is (C₁-C₆)alkylenyl or 4- to 6-membered heterocycloalkylenyl substituted with 0, 1, 2, 3 or 4 of R₇;
each R₇ is halogen, hydroxy, (C₁-C₆)alkyl, (C₁-C₆)alkoxy or halo(C₁-C₆)alkyl;
Q is represented by formula Q1, Q2 or Q3: 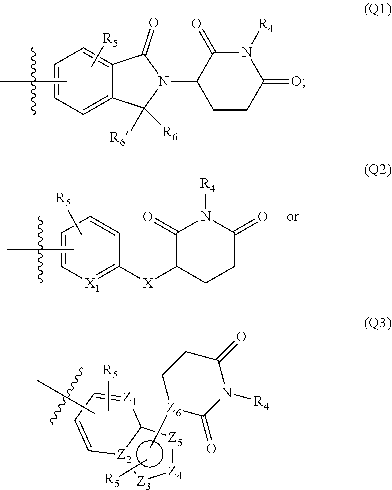
wherein,
X is a bond, O, —(CO)—NH— or NH;
X₁ is N or CH;
each Z₁ and Z₆ is independently C, CH or N;
each Z₂, Z₃ Z₄ and Z₅ is independently C, CH or N;
each R₄ is hydrogen or (C₁-C₆)alkyl;
each R₅ is hydrogen, halogen, halo(C₁-C₆)alkyl or (C₁-C₆)alkyl;
R₆ and R₆′ independently is hydrogen; or R₆ and R₆′ together represent an oxo group; and
‘n’ and ‘m’ are an integer, independently selected from 0, 1, 2 and 3.

2: The compound of claim 1, wherein R₁ and R₃ independently is halogen, (C₁-C₆)alkyl, (C₁-C₆)alkoxy, halo(C₁-C₆)alkyl, amino, 3- to 8-membered cycloalkyl, or hydroxy.

3: The compound of claim 1, wherein,
L¹ is a bond, phenylenyl or 4- to 6-membered heterocycloalkylenyl; wherein phenylenyl and heterocycloalkylenyl, at each occurrence, independently is substituted with 0, 1, 2, 3 or 4 of R₇;
L² is —CH₂— or 4- to 6-membered heterocycloalkylenyl substituted with 0, 1, 2, 3 or 4 of R₇; and
L³ is a bond, alkylenyl or 4- to 6-membered heterocycloalkylenyl; wherein the alkylenyl, and the heterocycloalkylenyl, at each occurrence, is substituted with 0, 1, 2, 3 or 4 of R⁷.

4: The compound of claim 1, wherein Q is represented by formula Q1: 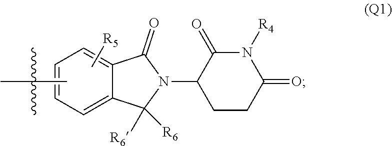
wherein
R₄ is hydrogen or (C₁-C₆)alkyl;
R₅ is hydrogen; and
R₆ and R₆′ independently is hydrogen; or R₆ and R₆′ together represent an oxo group.

5: The compound of claim 1, wherein Q is represented by formula Q2: 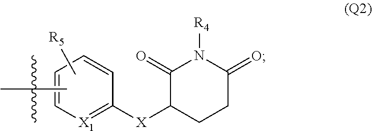
wherein
X is a bond, O, —(CO)—NH— or NH;
X₁ is N or CH;
R₅ is hydrogen or halogen; and
R₄ is hydrogen or (C₁-C₆)alkyl.

6: The compound of claim 1, wherein Q is represented by formula Q3: 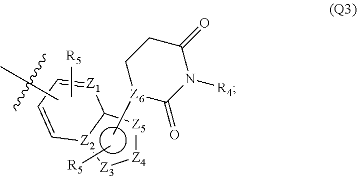
wherein,
R₄ is hydrogen or (C₁-C₆)alkyl;
R₅ is hydrogen or halogen;
each Z₁ and Z₆ is independently C, CH or N; and
each Z₂, Z₃ Z₄ and Z₅ is independently C, CH or N.

7: The compound of claim 1, wherein Q represents

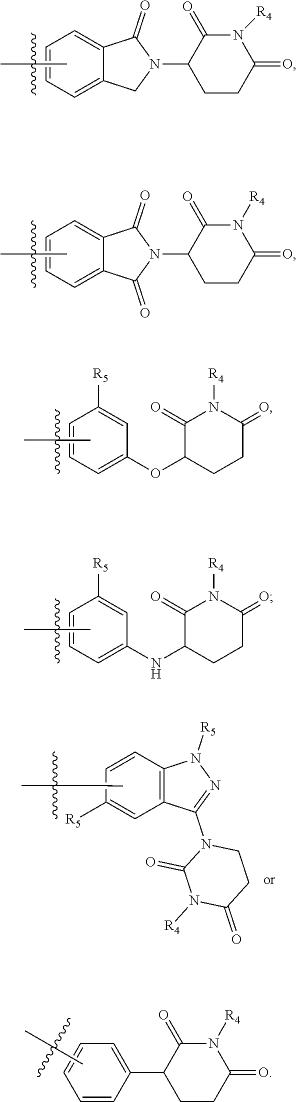

8: The compound of claim 1, having the compound of formula (IA):

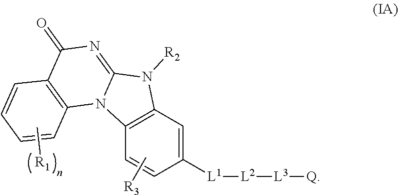

9-15. (canceled)

16: The compound claim 8,
wherein
R₁ and R₃ independently is halogen, (C₁-C₆)alkyl, (C₁-C₆)alkoxy, halo(C₁-C₆)alkyl, amino, cyclopropyl, cyclobutyl, cyclopentyl or hydroxy;
R₂ is hydrogen, halogen, hydroxy, (C₁-C₆)alkyl or 3- to 8-membered cycloalkyl;
L¹ is a bond, phenylenyl or 4- to 6-membered heterocycloalkylenyl;
L² is —CH₂— or 4- to 6-membered heterocycloalkylenyl;
L³ is a bond, alkylenyl or 4- to 6-membered heterocycloalkylenyl; 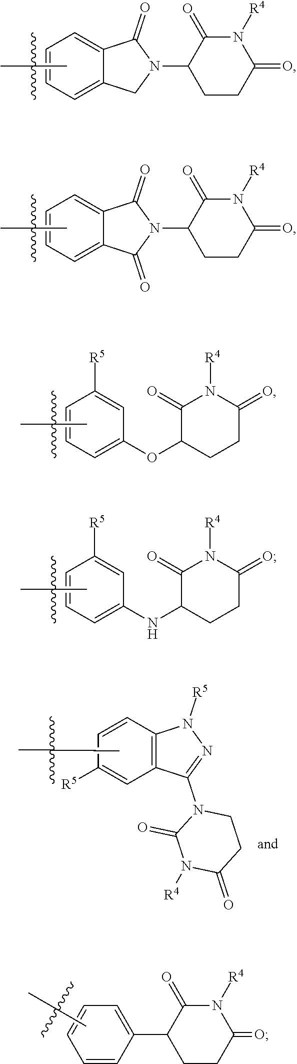
Q is selected from
wherein each R₄ is (C₁-C₆)alkyl or hydrogen; and each R₅ is (C₁-C₆)alkyl or hydrogen.

17: The compound of claim 1, having the compound of formula (IB): 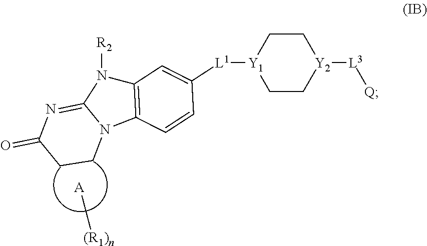
wherein Y₁ and Y₂ independently is CH or N;
L¹ is a bond, phenylenyl or heterocycloalkylenyl; and
L³ is a bond, alkylenyl or heterocycloalkylenyl.

18-21. (canceled)

22: The compound of claim 17, wherein
A is phenyl or pyridyl;
each R₁ is halogen, (C₁-C₆)alkyl, (C₁-C₆)alkoxy, halo(C₁-C₆)alkyl, amino or hydroxy;
Y₁ and Y₂ independently is CH or N;
R₂ is hydrogen, halogen, hydroxy, (C₁-C₆)alkyl or 3- to 8-membered cycloalkyl;
L₁ is a bond or a group selected from 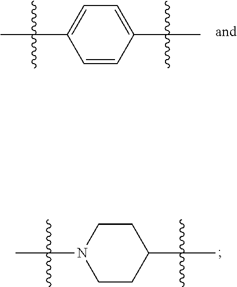 [SMILES: *c1ccc(*)cc1 ; *C1CCN(*)CC1]
L₃ is a bond or a group selected from —CH₂—, —CH₂—CH₂—, —CH(CH₃)—CH₂—, 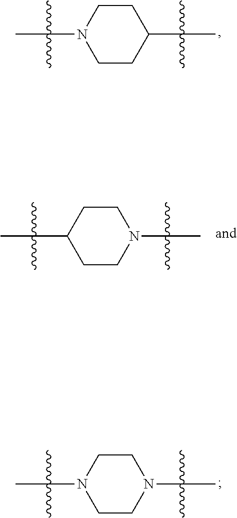 [SMILES: *C1CCN(*)CC1 ; *C1CCN(*)CC1 ; *N1CCN(*)CC1]
Q represents 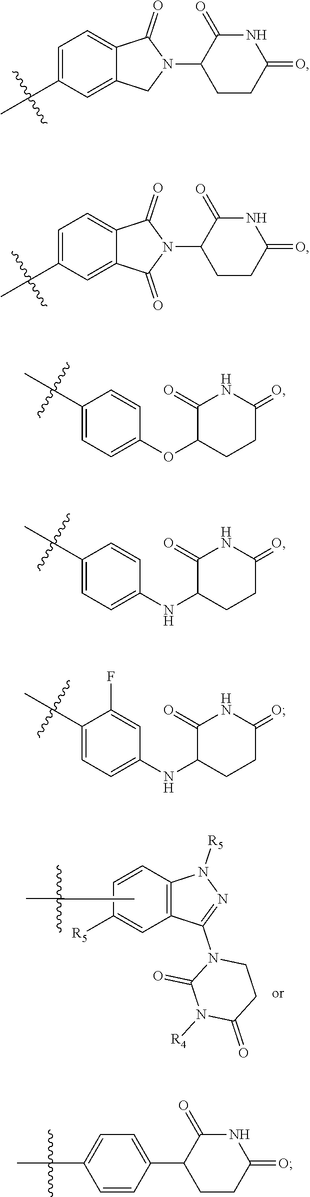
and
‘n’ is an integer selected from 0, 1 and 2.

23: The compound of claim 1, having the compound of formula (IC): 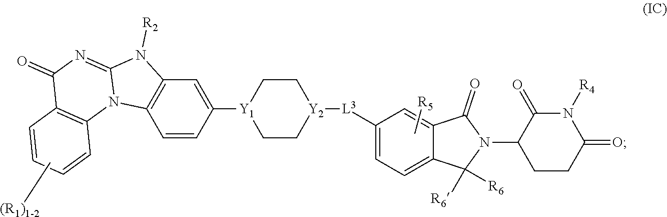
wherein Y₁ and Y₂ independently is CH or N; and L³ is (C₁-C₆)alkylenyl or 6-membered heterocycloalkylenyl.

24. (canceled)

25: The compound of claim 23, wherein Y₁ and Y₂ independently is CH or N;
L³ is selected from —CH₂, —CH₂CH₂—, 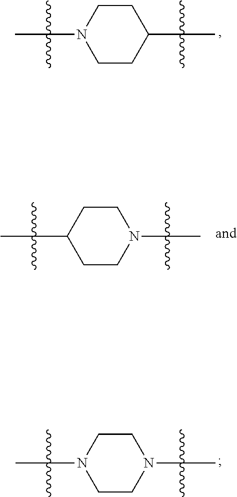 [SMILES: *C1CCN(*)CC1 ; *C1CCN(*)CC1 ; *N1CCN(*)CC1]
R₁ is selected from —Br, —Cl, —NH₂, —CH₃, —OCH₃, —OH and —CF₃;
R₂ is hydrogen, —CH₃, —CH(CH₃)₂, cyclopropyl, cyclobutyl or cyclopentyl; and 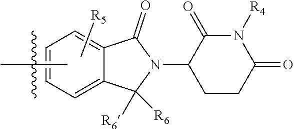
represents

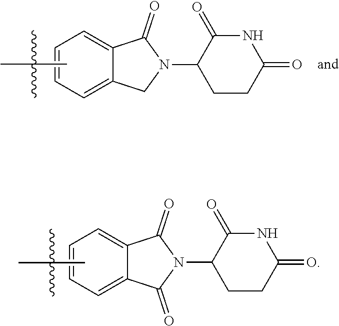

26: The compound of claim 1, having the compound of formula (ID): 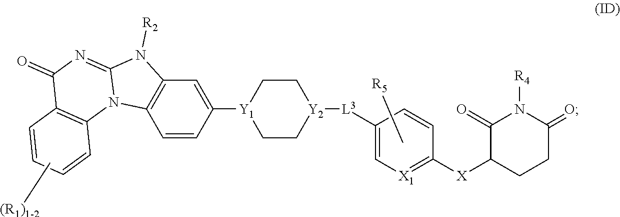
wherein Y₁ and Y₂ independently is CH or N; and L³ is (C₁-C₆)alkylenyl or 6-membered heterocycloalkylenyl.

27. (canceled)

28: The compound of claim 26, wherein
Y₁ and Y₂ independently is CH or N;
L³ is selected from —CH₂, —CH₂CH₂—, 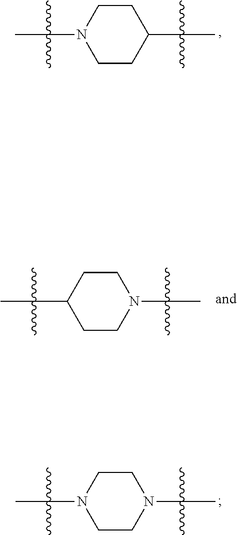 [SMILES: *C1CCN(*)CC1 ; *C1CCN(*)CC1 ; *N1CCN(*)CC1]
R₁ is selected from —Br, —Cl, —NH₂, —CH₃, —OCH₃, —OH and —CF₃;
R₂ is hydrogen, —CH₃, —CH(CH₃)₂, cyclopropyl, cyclobutyl or cyclopentyl; and 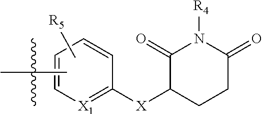
represents

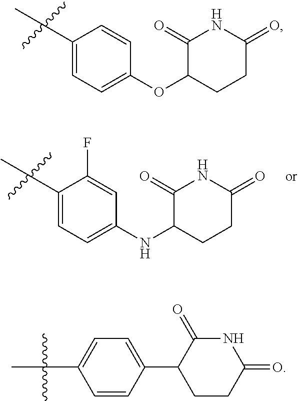 [SMILES: *c1ccc(OC2CCC(=O)NC2=O)cc1 ; *c1ccc(NC2CCC(=O)NC2=O)cc1F ; *c1ccc(C2CCC(=O)NC2=O)cc1]

29: The compound of claim 1, is selected from:

| Compound | |
|---|---|
| No. | Structure |
| 1 | 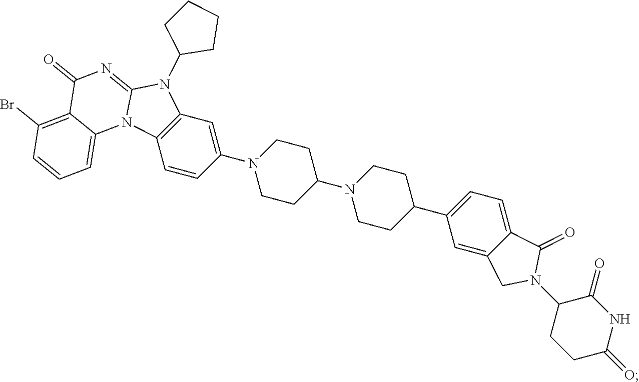 [SMILES: O=C1CCC(N2Cc3cc(C4CCN(C5CCN(c6ccc7c(c6)n(C6CCCC6)c6nc(=O)c8c(Br)cccc8n76)CC5)CC4)ccc3C2=O)C(=O)N1] |
| 2 | 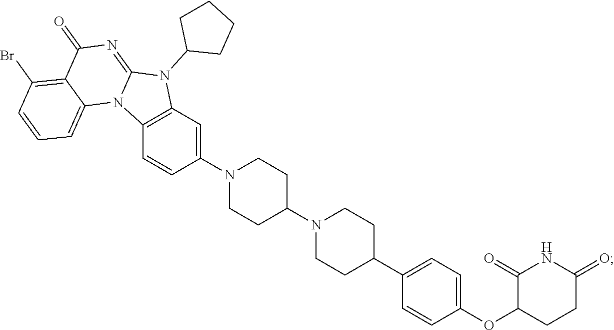 [SMILES: O=C1CCC(Oc2ccc(C3CCN(C4CCN(c5ccc6c(c5)n(C5CCCC5)c5nc(=O)c7c(Br)cccc7n65)CC4)CC3)cc2)C(=O)N1] |
| 3 | 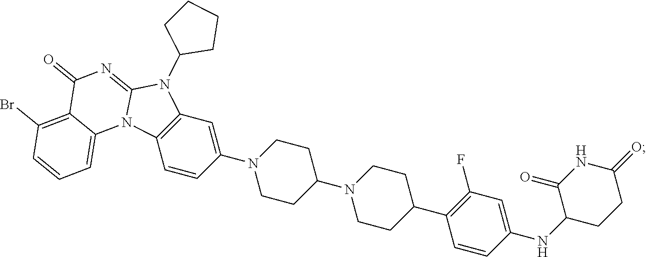 [SMILES: O=C1CCC(Nc2ccc(C3CCN(C4CCN(c5ccc6c(c5)n(C5CCCC5)c5nc(=O)c7c(Br)cccc7n65)CC4)CC3)c(F)c2)C(=O)N1] |
| 4 | 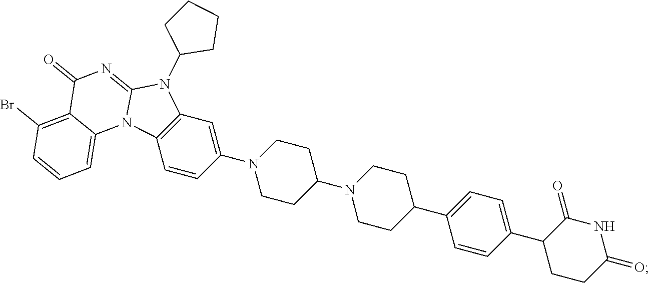 [SMILES: O=C1CCC(c2ccc(C3CCN(C4CCN(c5ccc6c(c5)n(C5CCCC5)c5nc(=O)c7c(Br)cccc7n65)CC4)CC3)cc2)C(=O)N1] |
| 5 | 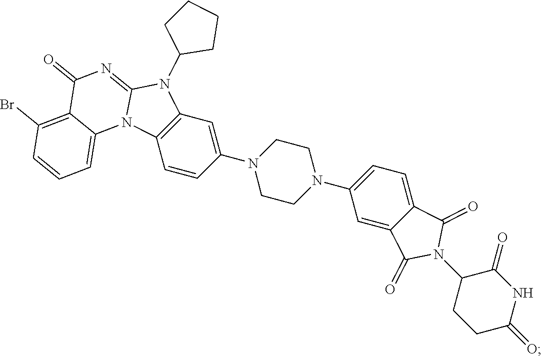 [SMILES: O=C1CCC(N2C(=O)c3ccc(N4CCN(c5ccc6c(c5)n(C5CCCC5)c5nc(=O)c7c(Br)cccc7n65)CC4)cc3C2=O)C(=O)N1] |
| 6 | 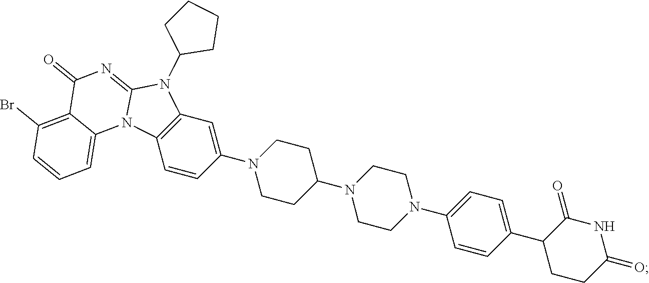 [SMILES: O=C1CCC(c2ccc(N3CCN(C4CCN(c5ccc6c(c5)n(C5CCCC5)c5nc(=O)c7c(Br)cccc7n65)CC4)CC3)cc2)C(=O)N1] |
| 7 | 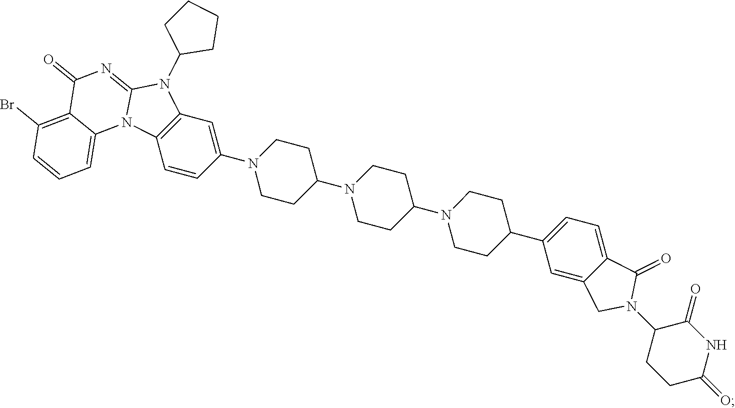 [SMILES: O=C1CCC(N2Cc3cc(C4CCN(C5CCN(C6CCN(c7ccc8c(c7)n(C7CCCC7)c7nc(=O)c9c(Br)cccc9n87)CC6)CC5)CC4)ccc3C2=O)C(=O)N1] |
| 8 | 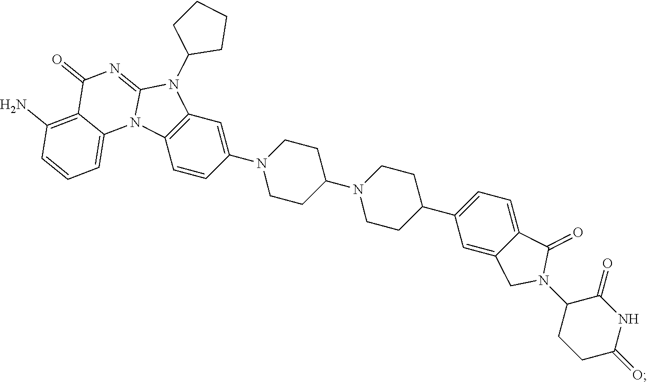 [SMILES: Nc1cccc2c1c(=O)nc1n(C3CCCC3)c3cc(N4CCC(N5CCC(c6ccc7c(c6)CN(C6CCC(=O)NC6=O)C7=O)CC5)CC4)ccc3n21] |
| 9 | 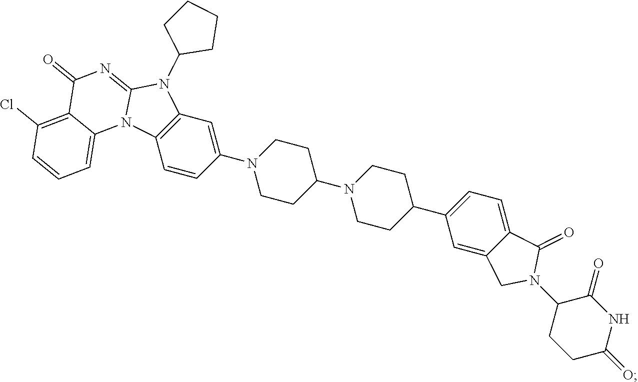 [SMILES: O=C1CCC(N2Cc3cc(C4CCN(C5CCN(c6ccc7c(c6)n(C6CCCC6)c6nc(=O)c8c(Cl)cccc8n76)CC5)CC4)ccc3C2=O)C(=O)N1] |
| 10 | 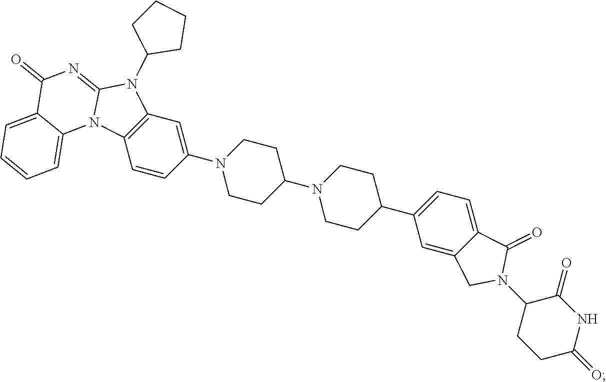 [SMILES: O=C1CCC(N2Cc3cc(C4CCN(C5CCN(c6ccc7c(c6)n(C6CCCC6)c6nc(=O)c8ccccc8n76)CC5)CC4)ccc3C2=O)C(=O)N1] |
| 11 | 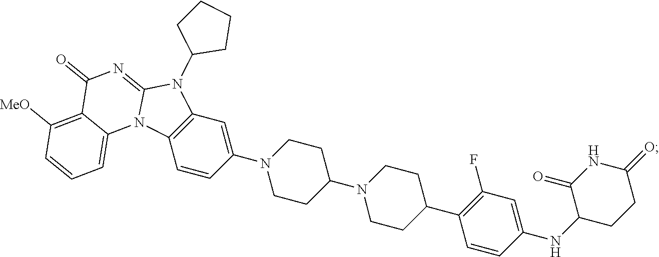 [SMILES: COc1cccc2c1c(=O)nc1n(C3CCCC3)c3cc(N4CCC(N5CCC(c6ccc(NC7CCC(=O)NC7=O)cc6F)CC5)CC4)ccc3n21] |
| 12 | 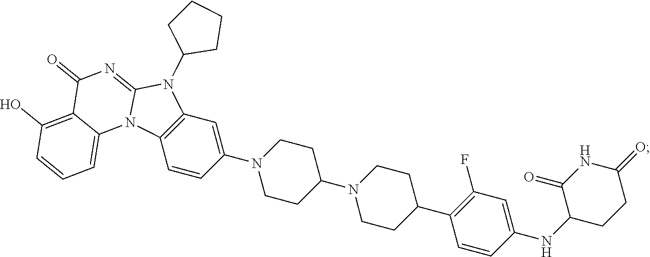 [SMILES: O=C1CCC(Nc2ccc(C3CCN(C4CCN(c5ccc6c(c5)n(C5CCCC5)c5nc(=O)c7c(O)cccc7n65)CC4)CC3)c(F)c2)C(=O)N1] |
| 13 | 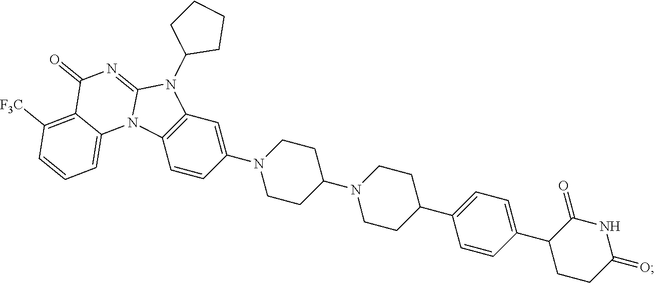 [SMILES: O=C1CCC(c2ccc(C3CCN(C4CCN(c5ccc6c(c5)n(C5CCCC5)c5nc(=O)c7c(C(F)(F)F)cccc7n65)CC4)CC3)cc2)C(=O)N1] |
| 14 | 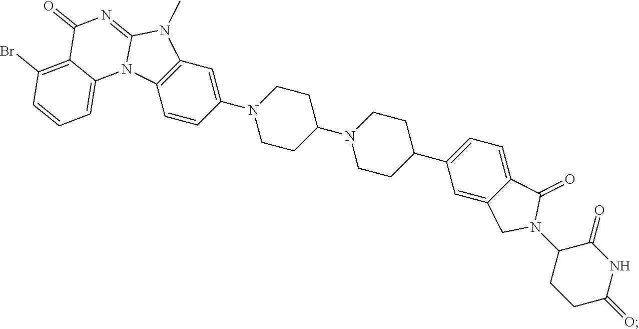 [SMILES: Cn1c2cc(N3CCC(N4CCC(c5ccc6c(c5)CN(C5CCC(=O)NC5=O)C6=O)CC4)CC3)ccc2n2c3cccc(Br)c3c(=O)nc12] |
| 15 | 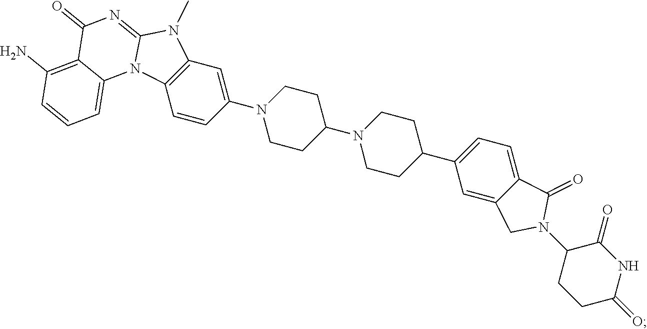 [SMILES: Cn1c2cc(N3CCC(N4CCC(c5ccc6c(c5)CN(C5CCC(=O)NC5=O)C6=O)CC4)CC3)ccc2n2c3cccc(N)c3c(=O)nc12] |
| 16 | 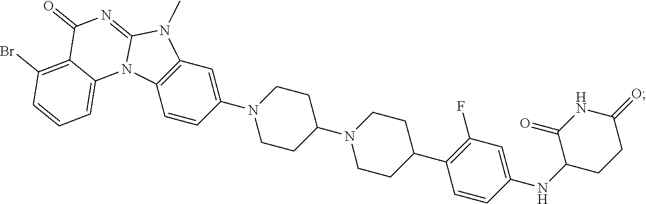 [SMILES: Cn1c2cc(N3CCC(N4CCC(c5ccc(NC6CCC(=O)NC6=O)cc5F)CC4)CC3)ccc2n2c3cccc(Br)c3c(=O)nc12] |
| 17 | 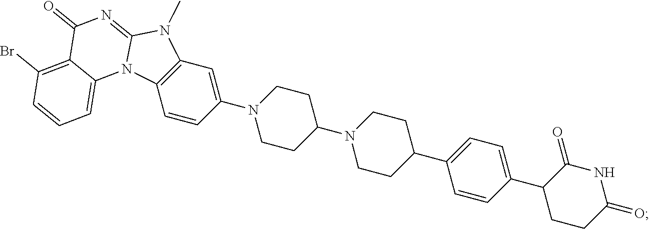 [SMILES: Cn1c2cc(N3CCC(N4CCC(c5ccc(C6CCC(=O)NC6=O)cc5)CC4)CC3)ccc2n2c3cccc(Br)c3c(=O)nc12] |
| 18 | 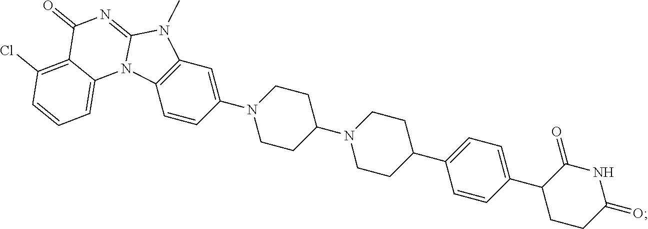 [SMILES: Cn1c2cc(N3CCC(N4CCC(c5ccc(C6CCC(=O)NC6=O)cc5)CC4)CC3)ccc2n2c3cccc(Cl)c3c(=O)nc12] |
| 19 | 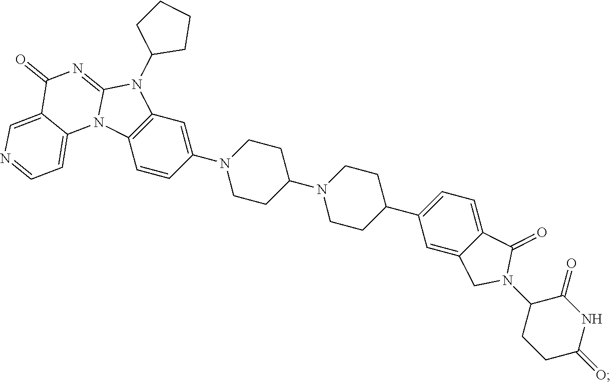 [SMILES: O=C1CCC(N2Cc3cc(C4CCN(C5CCN(c6ccc7c(c6)n(C6CCCC6)c6nc(=O)c8cnccc8n76)CC5)CC4)ccc3C2=O)C(=O)N1] |
| 20 | 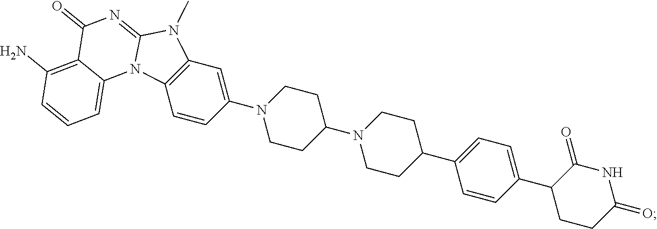 [SMILES: Cn1c2cc(N3CCC(N4CCC(c5ccc(C6CCC(=O)NC6=O)cc5)CC4)CC3)ccc2n2c3cccc(N)c3c(=O)nc12] |
| 21 | 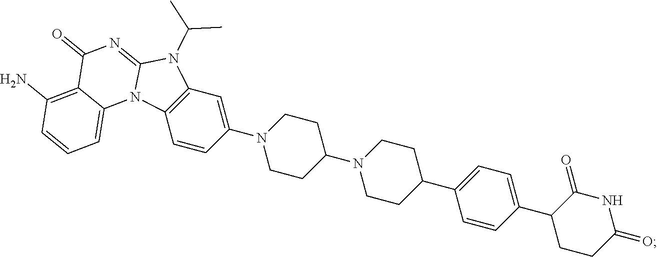 [SMILES: CC(C)n1c2cc(N3CCC(N4CCC(c5ccc(C6CCC(=O)NC6=O)cc5)CC4)CC3)ccc2n2c3cccc(N)c3c(=O)nc12] |
| 22 | 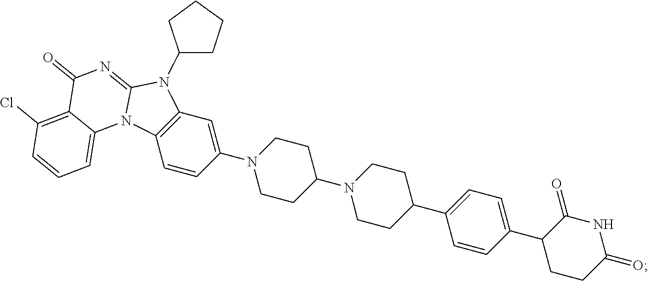 [SMILES: O=C1CCC(c2ccc(C3CCN(C4CCN(c5ccc6c(c5)n(C5CCCC5)c5nc(=O)c7c(Cl)cccc7n65)CC4)CC3)cc2)C(=O)N1] |
| 23 | 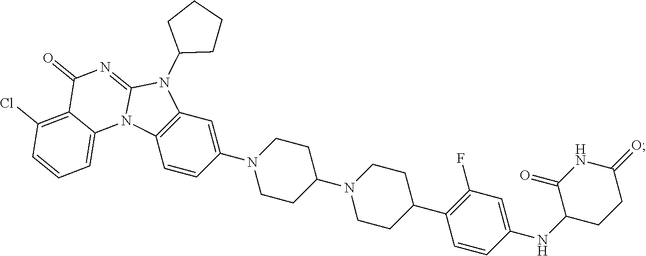 [SMILES: O=C1CCC(Nc2ccc(C3CCN(C4CCN(c5ccc6c(c5)n(C5CCCC5)c5nc(=O)c7c(Cl)cccc7n65)CC4)CC3)c(F)c2)C(=O)N1] |
| 24 | 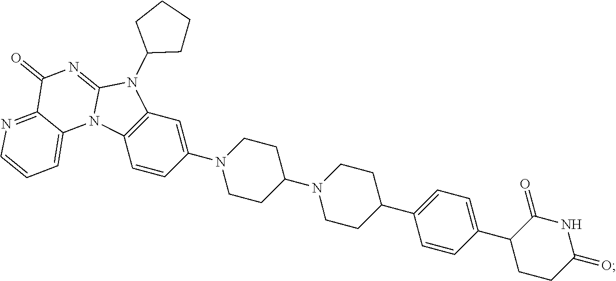 [SMILES: O=C1CCC(c2ccc(C3CCN(C4CCN(c5ccc6c(c5)n(C5CCCC5)c5nc(=O)c7ncccc7n65)CC4)CC3)cc2)C(=O)N1] |
| 25 | 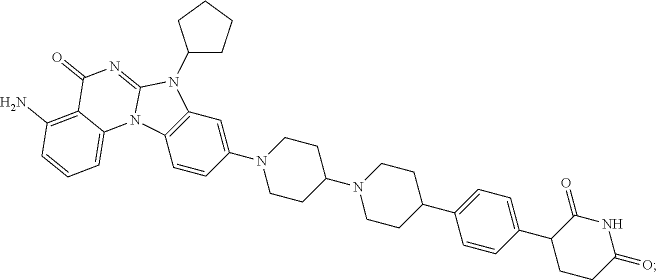 [SMILES: Nc1cccc2c1c(=O)nc1n(C3CCCC3)c3cc(N4CCC(N5CCC(c6ccc(C7CCC(=O)NC7=O)cc6)CC5)CC4)ccc3n21] |
| 26 | 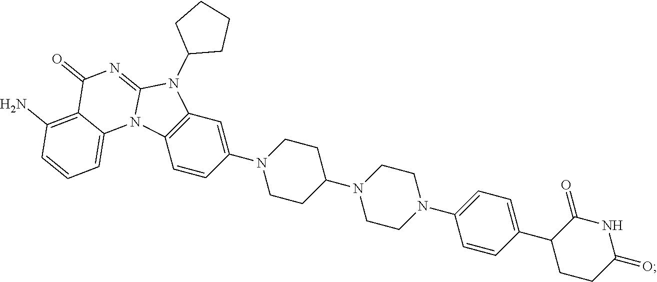 [SMILES: Nc1cccc2c1c(=O)nc1n(C3CCCC3)c3cc(N4CCC(N5CCN(c6ccc(C7CCC(=O)NC7=O)cc6)CC5)CC4)ccc3n21] |
| 27 | 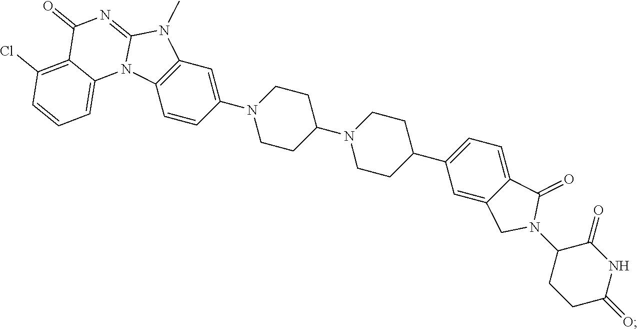 [SMILES: Cn1c2cc(N3CCC(N4CCC(c5ccc6c(c5)CN(C5CCC(=O)NC5=O)C6=O)CC4)CC3)ccc2n2c3cccc(Cl)c3c(=O)nc12] |
| 28 | 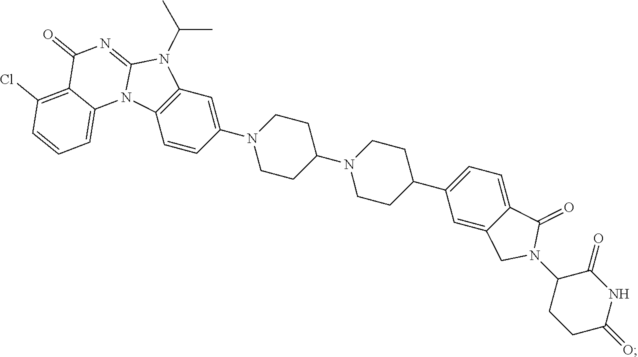 [SMILES: CC(C)n1c2cc(N3CCC(N4CCC(c5ccc6c(c5)CN(C5CCC(=O)NC5=O)C6=O)CC4)CC3)ccc2n2c3cccc(Cl)c3c(=O)nc12] |
| 29 | 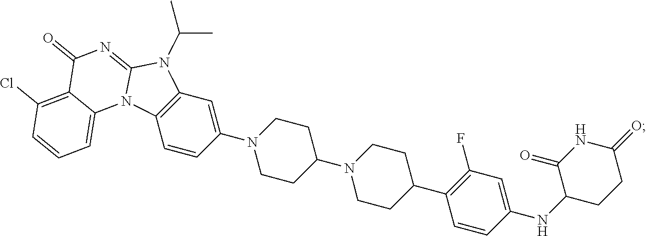 [SMILES: CC(C)n1c2cc(N3CCC(N4CCC(c5ccc(NC6CCC(=O)NC6=O)cc5F)CC4)CC3)ccc2n2c3cccc(Cl)c3c(=O)nc12] |
| 30 | 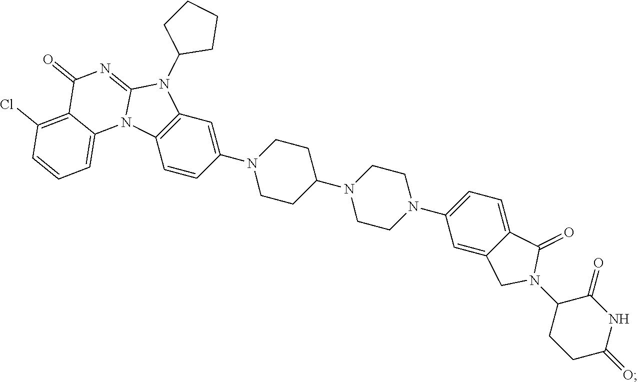 [SMILES: O=C1CCC(N2Cc3cc(N4CCN(C5CCN(c6ccc7c(c6)n(C6CCCC6)c6nc(=O)c8c(Cl)cccc8n76)CC5)CC4)ccc3C2=O)C(=O)N1] |
| 31 | 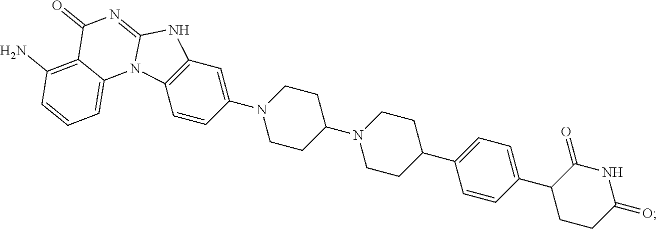 [SMILES: Nc1cccc2c1c(=O)nc1[nH]c3cc(N4CCC(N5CCC(c6ccc(C7CCC(=O)NC7=O)cc6)CC5)CC4)ccc3n12] |
| 32 | 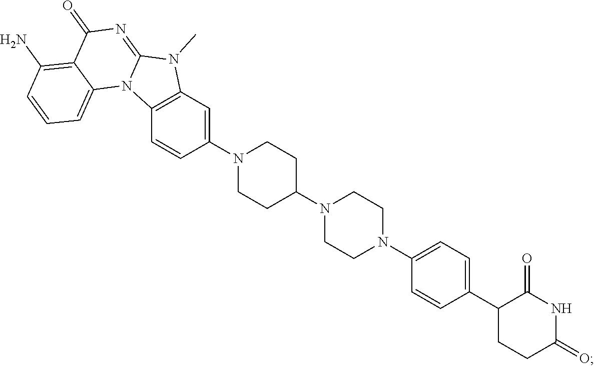 [SMILES: Cn1c2cc(N3CCC(N4CCN(c5ccc(C6CCC(=O)NC6=O)cc5)CC4)CC3)ccc2n2c3cccc(N)c3c(=O)nc12] |
| 33 | 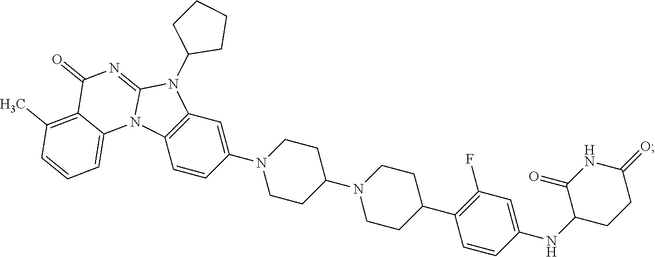 [SMILES: Cc1cccc2c1c(=O)nc1n(C3CCCC3)c3cc(N4CCC(N5CCC(c6ccc(NC7CCC(=O)NC7=O)cc6F)CC5)CC4)ccc3n21] |
| 34 | 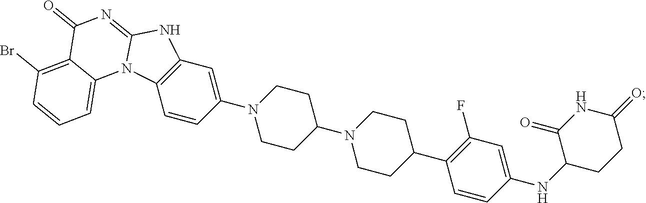 [SMILES: O=C1CCC(Nc2ccc(C3CCN(C4CCN(c5ccc6c(c5)[nH]c5nc(=O)c7c(Br)cccc7n56)CC4)CC3)c(F)c2)C(=O)N1] |
| 35 | 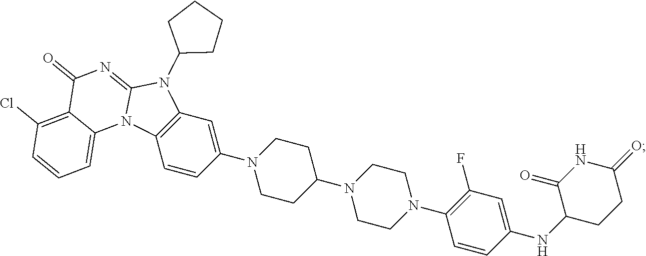 [SMILES: O=C1CCC(Nc2ccc(N3CCN(C4CCN(c5ccc6c(c5)n(C5CCCC5)c5nc(=O)c7c(Cl)cccc7n65)CC4)CC3)c(F)c2)C(=O)N1] |
| 36 | 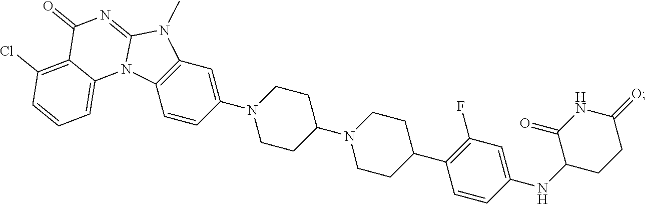 [SMILES: Cn1c2cc(N3CCC(N4CCC(c5ccc(NC6CCC(=O)NC6=O)cc5F)CC4)CC3)ccc2n2c3cccc(Cl)c3c(=O)nc12] |
| 37 | 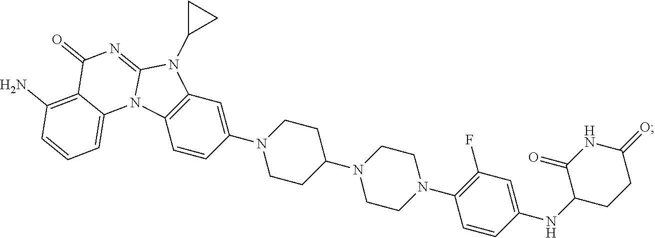 [SMILES: Nc1cccc2c1c(=O)nc1n(C3CC3)c3cc(N4CCC(N5CCN(c6ccc(NC7CCC(=O)NC7=O)cc6F)CC5)CC4)ccc3n21] |
| 38 | 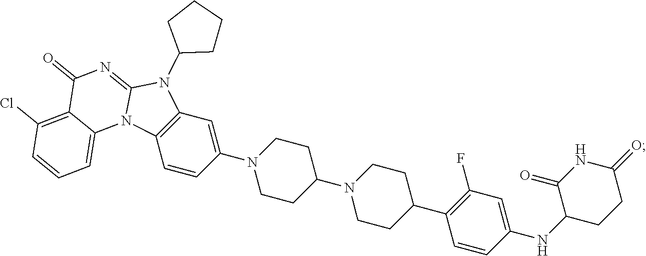 [SMILES: O=C1CCC(Nc2ccc(C3CCN(C4CCN(c5ccc6c(c5)n(C5CCCC5)c5nc(=O)c7c(Cl)cccc7n65)CC4)CC3)c(F)c2)C(=O)N1] |
| 39 | 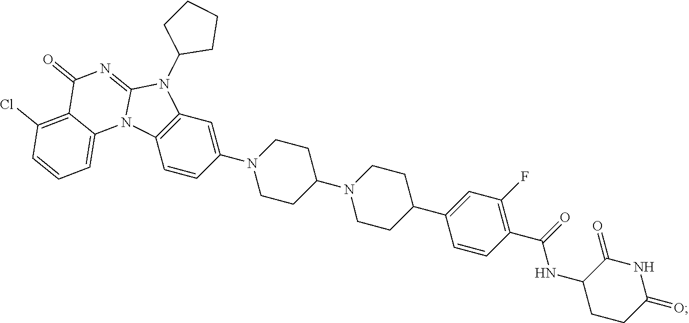 [SMILES: O=C1CCC(NC(=O)c2ccc(C3CCN(C4CCN(c5ccc6c(c5)n(C5CCCC5)c5nc(=O)c7c(Cl)cccc7n65)CC4)CC3)cc2F)C(=O)N1] |
| 40 | 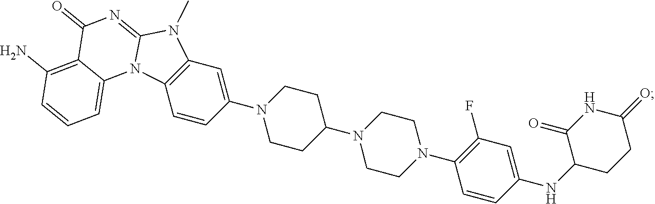 [SMILES: Cn1c2cc(N3CCC(N4CCN(c5ccc(NC6CCC(=O)NC6=O)cc5F)CC4)CC3)ccc2n2c3cccc(N)c3c(=O)nc12] |
| 41 | 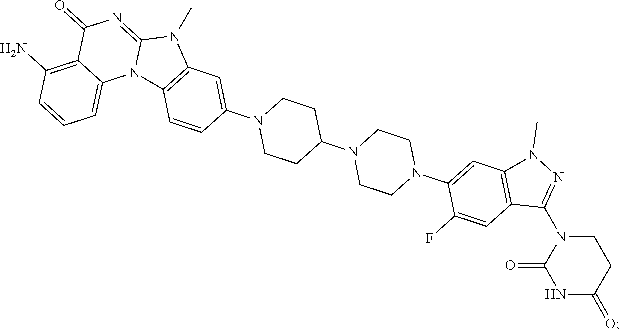 [SMILES: Cn1nc(N2CCC(=O)NC2=O)c2cc(F)c(N3CCN(C4CCN(c5ccc6c(c5)n(C)c5nc(=O)c7c(N)cccc7n65)CC4)CC3)cc21] |
| 42 | 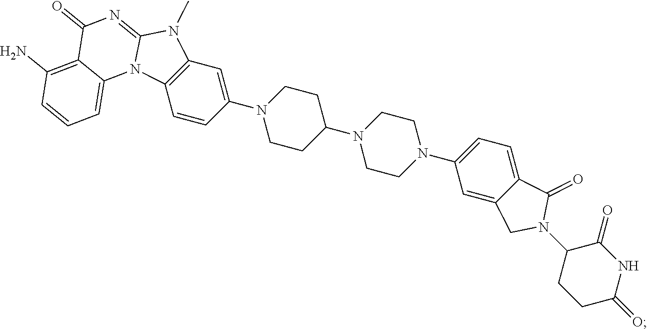 [SMILES: Cn1c2cc(N3CCC(N4CCN(c5ccc6c(c5)CN(C5CCC(=O)NC5=O)C6=O)CC4)CC3)ccc2n2c3cccc(N)c3c(=O)nc12] |
| 43 | 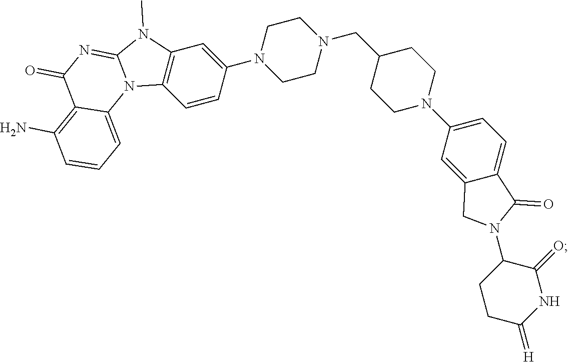 |
| 44 | 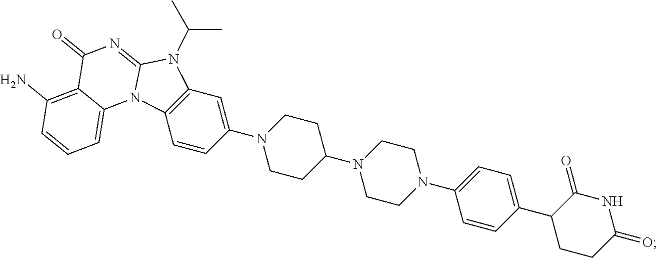 [SMILES: CC(C)n1c2cc(N3CCC(N4CCN(c5ccc(C6CCC(=O)NC6=O)cc5)CC4)CC3)ccc2n2c3cccc(N)c3c(=O)nc12] |
| 45 | 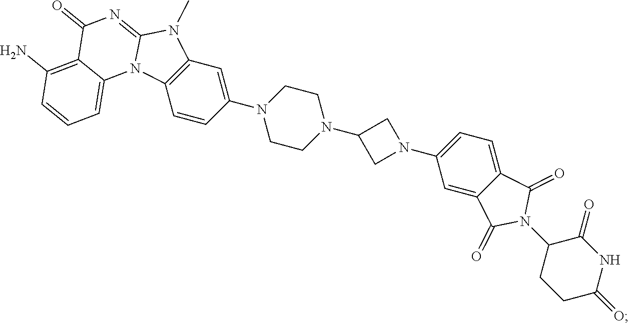 [SMILES: Cn1c2cc(N3CCN(C4CN(c5ccc6c(c5)C(=O)N(C5CCC(=O)NC5=O)C6=O)C4)CC3)ccc2n2c3cccc(N)c3c(=O)nc12] |
| 46 | 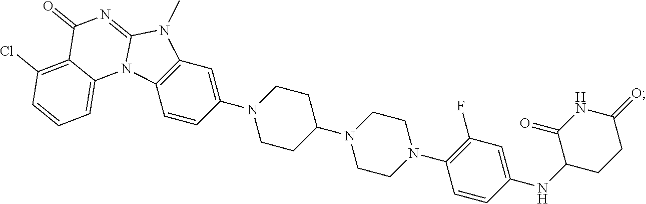 [SMILES: Cn1c2cc(N3CCC(N4CCN(c5ccc(NC6CCC(=O)NC6=O)cc5F)CC4)CC3)ccc2n2c3cccc(Cl)c3c(=O)nc12] |
| 47 | 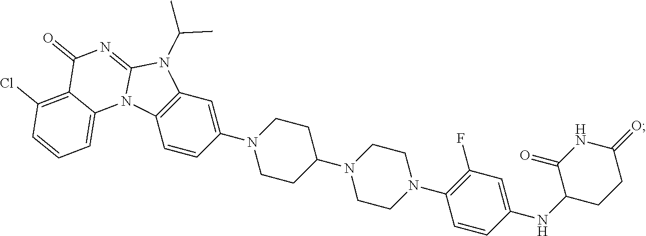 [SMILES: CC(C)n1c2cc(N3CCC(N4CCN(c5ccc(NC6CCC(=O)NC6=O)cc5F)CC4)CC3)ccc2n2c3cccc(Cl)c3c(=O)nc12] |
| 48 | 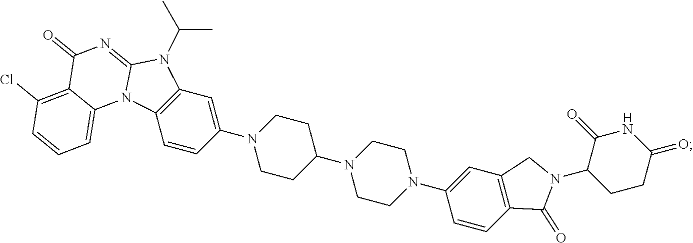 [SMILES: CC(C)n1c2cc(N3CCC(N4CCN(c5ccc6c(c5)CN(C5CCC(=O)NC5=O)C6=O)CC4)CC3)ccc2n2c3cccc(Cl)c3c(=O)nc12] |
| 49 | 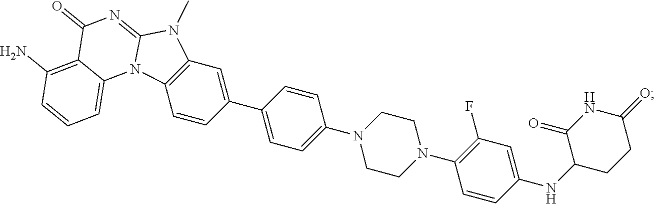 [SMILES: Cn1c2cc(-c3ccc(N4CCN(c5ccc(NC6CCC(=O)NC6=O)cc5F)CC4)cc3)ccc2n2c3cccc(N)c3c(=O)nc12] |
| | and |
| 50 | 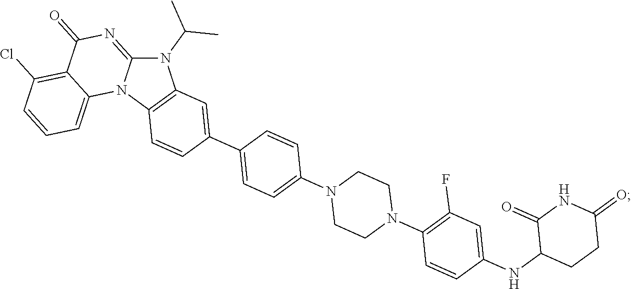 [SMILES: CC(C)n1c2cc(-c3ccc(N4CCN(c5ccc(NC6CCC(=O)NC6=O)cc5F)CC4)cc3)ccc2n2c3cccc(Cl)c3c(=O)nc12] |

or a pharmaceutically acceptable salt or a stereoisomer or a tautomer thereof.

30: A pharmaceutical composition comprising the compound of claim 1 or a pharmaceutically acceptable salt or a stereoisomer or a tautomer thereof, and a pharmaceutically acceptable carrier or an excipient.

31: A method for degrading a target protein in a subject, comprising administering to the subject a therapeutically effective amount of the pharmaceutical composition of claim 30; wherein the target protein is SMARCA2 and/or SMARCA4.

32-36. (canceled)

37: A method of treating or delaying progression of a disease or a disorder dependent upon at least one of SMARCA2 and SMARCA4 in a subject, comprising administering to the subject in need thereof, a therapeutically effective amount of the compound of claim 1 or a pharmaceutically acceptable salt or a stereoisomer or a tautomer thereof.

38: The method of claim 37, wherein the disease or disorder dependent upon SMARCA2 and/or SMARCA4 is cancer, selected from hematologic cancers, lung cancer, non-small cell lung cancer, acoustic neuroma, acute leukemia, acute lymphocytic acute myelocytic leukemia, adenocarcinoma, angiosarcoma, astrocytoma, myelomonocytic and promyelocytic, acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic, granulocytic leukemia, chronic myelogenous leukemia, colon cancer, colorectal cancer, craniopharyngioma, cystadenocarcinoma, diffuse large B-cell lymphoma, dysproliferative changes, dysplasias and metaplasias, embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, head and neck cancer, hemangioblastoma, hepatoma, hepatocellular cancer, hormone insensitive prostate cancer, leiomyosarcoma, leukemia, liposarcoma, liver cancer, lymphagioendotheliosarcoma, lymphangiosarcoma, lymphoblastic leukemia, lymphoma, Hodgkin's lymphoma, non-Hodgkin's lymphoma, Burkitt's lymphoma, malignancies and hyperproliferative disorders of the bladder, breast, colon, lung, ovaries, pancreas, prostate, skin and uterus, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, malignant rhabdoid tumor (MRT), rhabdomyosarcoma, sarcoma, sebaceous gland carcinoma, seminoma, skin cancer, small cell lung carcinoma, solid tumors, carcinomas, sarcomas, small cell lung cancer, stomach cancer, squamous cell carcinoma, synovioma, sweat gland carcinoma, thyroid cancer, Waldenstrom's macroglobulinemia, testicular tumors, uterine cancer or Wilms' tumor.

39-40. (canceled)
